SEQUENCE LISTING (381 sequences)

SEQ ID NO: 1 - protein, 8 aa, artificial sequence
  synthetic
GYAFSDYW

SEQ ID NO: 2 - protein, 8 aa, artificial sequence
  synthetic
ICPGSGNI

SEQ ID NO: 3 - protein, 10 aa, artificial sequence
  synthetic
TRLITRVIDY

SEQ ID NO: 4 - protein, 5 aa, artificial sequence
  synthetic
SSVNY

SEQ ID NO: 5 - protein, 3 aa, artificial sequence
  synthetic
DTS

SEQ ID NO: 6 - protein, 9 aa, artificial sequence
  synthetic
FQGSGYPLT

SEQ ID NO: 7 - protein, 117 aa, artificial sequence
  synthetic
EVQLQQSGAELVRPGTSVKISCKASGYAFSDYWLSWVKQRPGHGLEWIGDICPGSGNIYYNERFKDKVILTADRSSSTAYLQFSSLTSEDSVVYFCTRLITRVIDYWGQGTSVTVST

SEQ ID NO: 8 - protein, 106 aa, artificial sequence
  synthetic
ENVLTQSPAIMSASPGEKVTMTCSASSSVNYMHWYQQKSSTSPKLWIYDTSKLSSGVPGRFSGSGSGNSYSLTISSMEAEDVATYYCFQGSGYPLTFGAGTKLELK

SEQ ID NO: 9 - protein, 8 aa, artificial sequence
  synthetic
GFTLSDYY

SEQ ID NO: 10 - protein, 10 aa, artificial sequence
  synthetic
MRNKANGYTT

SEQ ID NO: 11 - protein, 11 aa, artificial sequence
  synthetic
VRDRDGRPFAY

SEQ ID NO: 12 - protein, 12 aa, artificial sequence
  synthetic
QSLLNSINQKNF

SEQ ID NO: 13 - protein, 3 aa, artificial sequence
  synthetic
WAS

SEQ ID NO: 14 - protein, 9 aa, artificial sequence
  synthetic
QNDYTYPLT

SEQ ID NO: 15 - protein, 120 aa, artificial sequence
  synthetic
EVQLVESGGGLVQPGGSLRLSCATSGFTLSDYYMSWVRQPPGKAPEWLGFMRNKANGYTTEYSASVRGRFTISRDTSQSIIYLQMNTLRAEDGATYYCVRDRDGRPFAYWGQGTLVTVSA

SEQ ID NO: 16 - protein, 113 aa, artificial sequence
  synthetic
DIVMTQSPSSLTVTAGEKVTMSCKSSQSLLNSINQKNFLTWYQQKPGQPPKLLIYWASTRESGVPDRFTGSGSGTDFTLTISSVQAEDLAIYYCQNDYTYPLTFGTGTKLELK

SEQ ID NO: 17 - protein, 8 aa, artificial sequence
  synthetic
GYTFTDYA

SEQ ID NO: 18 - protein, 8 aa, artificial sequence
  synthetic
INTYSGDT

SEQ ID NO: 19 - protein, 14 aa, artificial sequence
  synthetic
ARGGGNYPYWYFDV

SEQ ID NO: 20 - protein, 7 aa, artificial sequence
  synthetic
SSVSSSY

SEQ ID NO: 21 - protein, 3 aa, artificial sequence
  synthetic
STS

SEQ ID NO: 22 - protein, 9 aa, artificial sequence
  synthetic
QQFSVYPLT

SEQ ID NO: 23 - protein, 121 aa, artificial sequence
  synthetic
EVQLQQSGPELVRPGVSVKISCKGSGYTFTDYAMHWVKQSHAQSLEWIGIINTYSGDTNYNQKFKGKATMTVDKSSSTAYMGLARLTSEDSAIFYCARGGGNYPYWYFDVWGAGTTVTVSS

SEQ ID NO: 24 - protein, 108 aa, artificial sequence
  synthetic
ENVLTQSPAIMSASPGEKVTMTCRASSSVSSSYLHWYQQKSGDSPKLWIYSTSNLASGVPARFSGSGSGTSYSLTISSVEAEDAATYYCQQFSVYPLTFGAGTKLEIK

SEQ ID NO: 25 - protein, 8 aa, artificial sequence
  synthetic
GYSFTGYF

SEQ ID NO: 26 - protein, 8 aa, artificial sequence
  synthetic
INPNNGDT

SEQ ID NO: 27 - protein, 12 aa, artificial sequence
  synthetic
GRGGLPFGALDY

SEQ ID NO: 28 - protein, 12 aa, artificial sequence
  synthetic
QSLLYSRNQKNY

SEQ ID NO: 29 - protein, 3 aa, artificial sequence
  synthetic
WAS

SEQ ID NO: 30 - protein, 9 aa, artificial sequence
  synthetic
QQYHSYPYT

SEQ ID NO: 31 - protein, 119 aa, artificial sequence
  synthetic
EVQLQQSGPDLVKPGASVKISCKASGYSFTGYFMNWVKQRHGKSLEWIGYINPNNGDTIYNQKFKGKATLTVDKSSSTAHMELLSLTSEDSAVYYCGRGGLPFGALDYWGQGTSVTVSS

SEQ ID NO: 32 - protein, 113 aa, artificial sequence
  synthetic
DIVMSQSPSSLAVSVGEKVNMSCKSSQSLLYSRNQKNYLAWNQQKPGQSPKLLIYWASARESGVPDRFTGSGSGTDFTLTISSVKAEDLAAYYCQQYHSYPYTFGGGTKLELK

SEQ ID NO: 33 - protein, 8 aa, artificial sequence
  synthetic
GDSITSAY

SEQ ID NO: 34 - protein, 7 aa, artificial sequence
  synthetic
ISNSGST

SEQ ID NO: 35 - protein, 12 aa, artificial sequence
  synthetic
VRGERYYGYGAY

SEQ ID NO: 36 - protein, 13 aa, artificial sequence
  synthetic
KSLLHSNGNTYLY

SEQ ID NO: 37 - protein, 3 aa, artificial sequence
  synthetic
RMS

SEQ ID NO: 38 - protein, 9 aa, artificial sequence
  synthetic
MQHLEYPFT

SEQ ID NO: 39 - protein, 118 aa, artificial sequence
  synthetic
EVQLQESGPSLVKPSQTLSLTCSVTGDSITSAYWNWIRKFPGNKLEYMGYISNSGSTYYNPSLKSRISITRDTSKNQYYLQLNSVTTEDTGTYYCVRGERYYGYGAYWGQGTLVTVSA

SEQ ID NO: 40 - protein, 112 aa, artificial sequence
  synthetic
DIVMTQATPSVPVTPGESISISCRSSKSLLHSNGNTYLYWFLQRPGQSPQLLIYRMSNLASGVPDRFSGSGSGTAFTLRISRVEAEDVGVYYCMQHLEYPFTFGAGTKLELK

SEQ ID NO: 41 - protein, 9 aa, artificial sequence
  synthetic
GYSITSDYA

SEQ ID NO: 42 - protein, 6 aa, artificial sequence
  synthetic
SHSGST

SEQ ID NO: 43 - protein, 12 aa, artificial sequence
  synthetic
ARSLGRRWYFDV

SEQ ID NO: 44 - protein, 12 aa, artificial sequence
  synthetic
QSLLNSSTQKNY

SEQ ID NO: 45 - protein, 3 aa, artificial sequence
  synthetic
FAS

SEQ ID NO: 46 - protein, 9 aa, artificial sequence
  synthetic
QQHYIIPFT

SEQ ID NO: 47 - protein, 119 aa, artificial sequence
  synthetic
EVQLVESGPGLVKPSQSLSLTCTVTGYSITSDYAWNWIRQFPGNKLEWMGYISHSGSTSYNPSLKSRISITRDTSKNQFFLQLNSVTTEDTATYYCARSLGRRWYFDVWGAGTTVTVSS

SEQ ID NO: 48 - protein, 113 aa, artificial sequence
  synthetic
DIVMTQSPSSLAMSVGQKVTLSCKSSQSLLNSSTQKNYLAWYQQKPGQSPKLLVYFASTRDSGVPDRFIGSGSGTEFTLTISSVQAEDLADYFCQQHYIIPFTFGAGTKLEIK

SEQ ID NO: 49 - protein, 8 aa, artificial sequence
  synthetic
GFNIKDYP

SEQ ID NO: 50 - protein, 8 aa, artificial sequence
  synthetic
FDPDNGNV

SEQ ID NO: 51 - protein, 9 aa, artificial sequence
  synthetic
SSPYGAMDY

SEQ ID NO: 52 - protein, 5 aa, artificial sequence
  synthetic
SSVSY

SEQ ID NO: 53 - protein, 3 aa, artificial sequence
  synthetic
DTS

SEQ ID NO: 54 - protein, 9 aa, artificial sequence
  synthetic
QQGSSNPRT

SEQ ID NO: 55 - protein, 116 aa, artificial sequence
  synthetic
EVQLQESGPDLVRPGALVKLSCKSSGFNIKDYPIHWVRQRPEQGLEWIGWFDPDNGNVIYDPRFQGKASITADTSSNTAYLQLNSLTSEDTAVYYCSSPYGAMDYWGQGTSVTVSS

SEQ ID NO: 56 - protein, 106 aa, artificial sequence
  synthetic
QIVLTQSPEIMSASPGEKVTMTCSASSSVSYIHWYQQKSGTSPKRWIYDTSKLASGVPARFSGSGSGTSYSLTIRSMEAEDAATYYCQQGSSNPRTFGGGTKLEIK

SEQ ID NO: 57 - protein, 8 aa, artificial sequence
  synthetic
GYTFTSYD

SEQ ID NO: 58 - protein, 8 aa, artificial sequence
  synthetic
IFPGNGDT

SEQ ID NO: 59 - protein, 11 aa, artificial sequence
  synthetic
ARDLRLGTFDY

SEQ ID NO: 60 - protein, 5 aa, artificial sequence
  synthetic
SSVSY

SEQ ID NO: 61 - protein, 3 aa, artificial sequence
  synthetic
DTS

SEQ ID NO: 62 - protein, 9 aa, artificial sequence
  synthetic
QQGSSNPRT

SEQ ID NO: 63 - protein, 118 aa, artificial sequence
  synthetic
EVQLQESGPELVKPGTSVKLSCKASGYTFTSYDINWVRQRPEQGLEWIGWIFPGNGDTYYNEKFKGKATLTSDKSSSTAYMQLSRLTSEDSAVYFCARDLRLGTFDYWGQGTTLTVSS

SEQ ID NO: 64 - protein, 106 aa, artificial sequence
  synthetic
QIVLTQSPEIMSASPGEKVTMTCSASSSVSYIHWYQQKSGTSPKRWIYDTSKLASGVPARFSGSGSGTSYSLTIRSMEAEDAATYYCQQGSSNPRTFGGGTKLELK

SEQ ID NO: 65 - protein, 8 aa, artificial sequence
  synthetic
GYTFTDYA

SEQ ID NO: 66 - protein, 8 aa, artificial sequence
  synthetic
INTYSDNT

SEQ ID NO: 67 - protein, 16 aa, artificial sequence
  synthetic
ARGLLLRSQLGVYFDY

SEQ ID NO: 68 - protein, 5 aa, artificial sequence
  synthetic
SSVSY

SEQ ID NO: 69 - protein, 3 aa, artificial sequence
  synthetic
ATS

SEQ ID NO: 70 - protein, 9 aa, artificial sequence
  synthetic
QQWSSNPWT

SEQ ID NO: 71 - protein, 123 aa, artificial sequence
  synthetic
EVQLQESGPELVRPGVSVKISCKGSGYTFTDYAMHWVKQSHAKSLEWIGVINTYSDNTNYNRKFKGKATMTVDKSSSTAYMELARLTSEDSAIYYCARGLLLRSQLGVYFDYWGQGTTLTVSS

SEQ ID NO: 72 - protein, 106 aa, artificial sequence
  synthetic
QIVLSQSPAILSASPGEKVTMTCRASSSVSYMHWYQQKPGSSPKPWIYATSNLASGVPARFSGSGSGTSYSLTISRVEAEDAATYYCQQWSSNPWTFGGGTKLEIK

SEQ ID NO: 73 - protein, 8 aa, artificial sequence
  synthetic
GFTFSYYA

SEQ ID NO: 74 - protein, 8 aa, artificial sequence
  synthetic
INGDGSHT

SEQ ID NO: 75 - protein, 9 aa, artificial sequence
  synthetic
ASLLQLGPF

SEQ ID NO: 76 - protein, 6 aa, artificial sequence
  synthetic
QNVGTN

SEQ ID NO: 77 - protein, 3 aa, artificial sequence
  synthetic
SAS

SEQ ID NO: 78 - protein, 9 aa, artificial sequence
  synthetic
QQYNNYPLT

SEQ ID NO: 79 - protein, 116 aa, artificial sequence
  synthetic
EVQLQESGGGLVKPGGSLKLSCAASGFTFSYYAMSWVRQTPEKRLEWVATINGDGSHTYYPDSVRGRFTISRDNAKNTLYLQVSSLRSEDTAMYYCASLLQLGPFWGQGTTLTVSS

SEQ ID NO: 80 - protein, 107 aa, artificial sequence
  synthetic
DIVMTQSQKFMSTSVGDRVSVTCKASQNVGTNVAWYHQRPGQSPKPLIYSASYRYSGVPDRFTGSGSGTDFTLTISNVQSEDLAEYFCQQYNNYPLTFGSGTKLEIK

SEQ ID NO: 81 - protein, 8 aa, artificial sequence
  synthetic
GLTFSTNY

SEQ ID NO: 82 - protein, 8 aa, artificial sequence
  synthetic
IYGGNGGT

SEQ ID NO: 83 - protein, 12 aa, artificial sequence
  synthetic
TSHDYYGGPMDY

SEQ ID NO: 84 - protein, 6 aa, artificial sequence
  synthetic
QDISNY

SEQ ID NO: 85 - protein, 3 aa, artificial sequence
  synthetic
FTS

SEQ ID NO: 86 - protein, 9 aa, artificial sequence
  synthetic
QQTNTLPWT

SEQ ID NO: 87 - protein, 119 aa, artificial sequence
  synthetic
EVQLQESGAELVKPGASVKLSCKTSGLTFSTNYISWLKQKPGQSLEWIAWIYGGNGGTSYNQRFTGKAQLTVDTSSSTAYMQFSSLTTEDSAIYYCTSHDYYGGPMDYWGQGTSVTVSS

SEQ ID NO: 88 - protein, 107 aa, artificial sequence
  synthetic
DIQMTQTTSSLSASLGDRVTISCRASQDISNYLNWYQQKPDGTVKLLIYFTSRLHSGVPSRFSASGSGTDYSLTISNLEQEDIATYFCQQTNTLPWTFGGGTKLEIK

SEQ ID NO: 89 - protein, 8 aa, artificial sequence
  synthetic
GYTFTENT

SEQ ID NO: 90 - protein, 8 aa, artificial sequence
  synthetic
INPNTGGT

SEQ ID NO: 91 - protein, 14 aa, artificial sequence
  synthetic
ARRGTNWEHWYFDV

SEQ ID NO: 92 - protein, 5 aa, artificial sequence
  synthetic
SSVSY

SEQ ID NO: 93 - protein, 3 aa, artificial sequence
  synthetic
DTS

SEQ ID NO: 94 - protein, 9 aa, artificial sequence
  synthetic
QQWSSNPLT

SEQ ID NO: 95 - protein, 121 aa, artificial sequence
  synthetic
EVQLQQSGPELVKPGASVKISCKTSGYTFTENTMHWVKQSHGKRLEWIGGINPNTGGTTYNQKFKGKATLTVDKSSSTAYMELRSLTSEDSAVYYCARRGTNWEHWYFDVWGAGTTVTVSS

SEQ ID NO: 96 - protein, 106 aa, artificial sequence
  synthetic
QIVLTQSPAIMSASPGEKVTMTCSASSSVSYMHWYQQKSGTSPKRWIYDTSKLASGVPARFSGSGSGTSYSLTISSMEAEDAATYYCQQWSSNPLTFGAGTKLELK

SEQ ID NO: 97 - protein, 9 aa, artificial sequence
  synthetic
GYSITSDYA

SEQ ID NO: 98 - protein, 7 aa, artificial sequence
  synthetic
ISYSGST

SEQ ID NO: 99 - protein, 9 aa, artificial sequence
  synthetic
ARGGPWFAY

SEQ ID NO: 100 - protein, 11 aa, artificial sequence
  synthetic
QSLVHSNGNTY

SEQ ID NO: 101 - protein, 3 aa, artificial sequence
  synthetic
KVS

SEQ ID NO: 102 - protein, 9 aa, artificial sequence
  synthetic
SQSTHVPFT

SEQ ID NO: 103 - protein, 116 aa, artificial sequence
  synthetic
DVQLQESGPGLVKPSQSLSLTCTVTGYSITSDYAWNWIRQFPGNKLEWMGYISYSGSTSYNPSLKSRISITRDTSKNQFFLQLNSVTTEDTATYYCARGGPWFAYWGQGTLVTVSA

SEQ ID NO: 104 - protein, 112 aa, artificial sequence
  synthetic
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPFTFGSGTKLEIK

SEQ ID NO: 105 - protein, 8 aa, artificial sequence
  synthetic
GYTFTKYG

SEQ ID NO: 106 - protein, 8 aa, artificial sequence
  synthetic
IWTYTGKP

SEQ ID NO: 107 - protein, 12 aa, artificial sequence
  synthetic
ARDDGYYYTMNY

SEQ ID NO: 108 - protein, 11 aa, artificial sequence
  synthetic
QSLLHTNGDTY

SEQ ID NO: 109 - protein, 3 aa, artificial sequence
  synthetic
KVS

SEQ ID NO: 110 - protein, 9 aa, artificial sequence
  synthetic
SQSTHIPWT

SEQ ID NO: 111 - protein, 119 aa, artificial sequence
  synthetic
QIQLVQSGPEVKKPGETVKISCKASGYTFTKYGINWVKQAPGEGLKWMGWIWTYTGKPTYGDDFKGRFAFSLETSASTAYLQISDLKIEDMATYFCARDDGYYYTMNYWGQGTSVTVSS

SEQ ID NO: 112 - protein, 112 aa, artificial sequence
  synthetic
DIVMTQTPLSLPVSLGDQASISCRSSQSLLHTNGDTYLHWYLQKAGQPPKLLIYKVSNRFSGVPDRFSGSGSGTDFTLKISRVEAEDLGVYFCSQSTHIPWTFGGGTKLEIK

SEQ ID NO: 113 - protein, 8 aa, artificial sequence
  synthetic
GYSFTNYG

SEQ ID NO: 114 - protein, 7 aa, artificial sequence
  synthetic
NPYTGEP

SEQ ID NO: 115 - protein, 12 aa, artificial sequence
  synthetic
GRDDGYHYTMNY

SEQ ID NO: 116 - protein, 11 aa, artificial sequence
  synthetic
QSLIHSNGDTY

SEQ ID NO: 117 - protein, 3 aa, artificial sequence
  synthetic
KVS

SEQ ID NO: 118 - protein, 9 aa, artificial sequence
  synthetic
SQSTYVPWT

SEQ ID NO: 119 - protein, 119 aa, artificial sequence
  synthetic
QIQLVQSGPELKRPGETVKISCKASGYSFTNYGMNWVKQAPGKGLKWMGWINPYTGEPTYADDFKGRFAMSLETSASAAYLQINNLKNEDMATYFCGRDDGYHYTMNYWGQGTSVTVSS

SEQ ID NO: 120 - protein, 112 aa, artificial sequence
  synthetic
DVVMTQPPLSLPVSLGDQASISCRSSQSLIHSNGDTYLHWYLQKPGQSPKLLIYKVSIRFSGVPDRFSGSGSGTDFTLKISRVEAEDLGVYFCSQSTYVPWTFGGGTKLEIK

SEQ ID NO: 121 - protein, 8 aa, artificial sequence
  synthetic
GYTFTDHT

SEQ ID NO: 122 - protein, 8 aa, artificial sequence
  synthetic
ISTYYGNT

SEQ ID NO: 123 - protein, 18 aa, artificial sequence
  synthetic
ARASYGSGYGPYYWYFDV

SEQ ID NO: 124 - protein, 6 aa, artificial sequence
  synthetic
EKIYSY

SEQ ID NO: 125 - protein, 3 aa, artificial sequence
  synthetic
NAN

SEQ ID NO: 126 - protein, 9 aa, artificial sequence
  synthetic
QHHYGTPVT

SEQ ID NO: 127 - protein, 125 aa, artificial sequence
  synthetic
EVQLQESGPELVRPGVSVKISCKGSGYTFTDHTMHWVKESHAQSLEWIGVISTYYGNTNYNQNFKGKATLTVDKSSSTAYMELAGLTSEDSAIYYCARASYGSGYGPYYWYFDVWGAGTTVTVSS

SEQ ID NO: 128 - protein, 107 aa, artificial sequence
  synthetic
DIQMTQSPASLFASVGETVTITCRASEKIYSYLAWYFQKQGKSPQFLVYNANALAEGVPSRFSGSGSGTQFSLKINSLQPEDFGTYYCQHHYGTPVTFGAGSKLELK

SEQ ID NO: 129 - protein, 8 aa, artificial sequence
  synthetic
GFTFSTYA

SEQ ID NO: 130 - protein, 8 aa, artificial sequence
  synthetic
ISGGGIDT

SEQ ID NO: 131 - protein, 13 aa, artificial sequence
  synthetic
ARQGSKGWGAMDY

SEQ ID NO: 132 - protein, 5 aa, artificial sequence
  synthetic
SSVSF

SEQ ID NO: 133 - protein, 3 aa, artificial sequence
  synthetic
ETS

SEQ ID NO: 134 - protein, 9 aa, artificial sequence
  synthetic
QQWSSNPLT

SEQ ID NO: 135 - protein, 120 aa, artificial sequence
  synthetic
EVQLQESGGGLVKPGGSLKFSCAASGFTFSTYAMSWVRQTPEKRLEWVATISGGGIDTYYPDSVKGRFTISRDNAKNTLYLQMNSLRSEDTAMYYCARQGSKGWGAMDYWGQGTSVTVSS

SEQ ID NO: 136 - protein, 106 aa, artificial sequence
  synthetic
QIVLTQSPAIMSASPGEKVTMTCRASSSVSFMHWYQQKSGTSPKRWIYETSKLASGVPDRFSGSGSGTSYSLTISSMEAEDAATYYCQQWSSNPLTFGAGTKLELK

SEQ ID NO: 137 - protein, 8 aa, artificial sequence
  synthetic
GFTFSSYA

SEQ ID NO: 138 - protein, 6 aa, artificial sequence
  synthetic
ISGGGI

SEQ ID NO: 139 - protein, 13 aa, artificial sequence
  synthetic
ARQGSKGWDAMDY

SEQ ID NO: 140 - protein, 5 aa, artificial sequence
  synthetic
SSVSF

SEQ ID NO: 141 - protein, 3 aa, artificial sequence
  synthetic
ETS

SEQ ID NO: 142 - protein, 9 aa, artificial sequence
  synthetic
QQWSSNPLT

SEQ ID NO: 143 - protein, 112 aa, artificial sequence
  synthetic
EIGGLVKPGGSLKFSCAASGFTFSSYAMSWVRQTPEKRLEWVATISGGGIYYPDSVKGRFTISRDNAKNTLYLQMSSLRSEDTAMYYCARQGSKGWDAMDYWGQGTSVTVSS

SEQ ID NO: 144 - protein, 106 aa, artificial sequence; identical to SEQ ID NO: 136
  synthetic

SEQ ID NO: 145 - protein, 8 aa, artificial sequence
  synthetic
GYTFTDYT

SEQ ID NO: 146 - protein, 8 aa, artificial sequence
  synthetic
ISTYYGNT

SEQ ID NO: 147 - protein, 18 aa, artificial sequence
  synthetic
ARASYGSGYGPYYWYFDV

SEQ ID NO: 148 - protein, 6 aa, artificial sequence
  synthetic
EKIYSY

SEQ ID NO: 149 - protein, 3 aa, artificial sequence
  synthetic
NAN

SEQ ID NO: 150 - protein, 9 aa, artificial sequence
  synthetic
QHHYGTPVT

SEQ ID NO: 151 - protein, 125 aa, artificial sequence
  synthetic
EVQLQESGPELVRPGVSVKISCKGSGYTFTDYTMHWVKLSHAKSLEWIGVISTYYGNTNYNQKFKGKATLTVDKSSSTAYMELARLTSEDSAIYYCARASYGSGYGPYYWYFDVWGAGTTVTVSS

SEQ ID NO: 152 - protein, 107 aa, artificial sequence; identical to SEQ ID NO: 128
  synthetic

SEQ ID NO: 153 - protein, 8 aa, artificial sequence
  synthetic
GYTFTSYW

SEQ ID NO: 154 - protein, 8 aa, artificial sequence
  synthetic
IHPKSGTV

SEQ ID NO: 155 - protein, 14 aa, artificial sequence
  synthetic
ARGENYDYRPQFVY

SEQ ID NO: 156 - protein, 7 aa, artificial sequence
  synthetic
SQHSTYT

SEQ ID NO: 157 - protein, 7 aa, artificial sequence
  synthetic
LKKDGSH

SEQ ID NO: 158 - protein, 13 aa, artificial sequence
  synthetic
GVGDTIKEQFVYV

SEQ ID NO: 159 - protein, 121 aa, artificial sequence
  synthetic
QVQLQQPGAELVKPGTSVKLSCKASGYTFTSYWIHWVKQRPGQGLEWIGMIHPKSGTVDYNEKFKSKATLTADKSSNTAYMQLSSLTSEASVVYYCARGENYDYRPQFVYWGQGTLVTVSA

SEQ ID NO: 160 - protein, 115 aa, artificial sequence
  synthetic
QLVLTQSSSASFSLGASAKLTCTLSSQHSTYTIEWYQQQPLKPPKYVMELKKDGSHRTGDGIPDRFSGSSSGADRYLSISNIQPEDEAIYICGVGDTIKEQFVYVFGGGTKVTVL

SEQ ID NO: 161 - protein, 8 aa, artificial sequence
  synthetic
GDTFTSYW

SEQ ID NO: 162 - protein, 8 aa, artificial sequence
  synthetic
IHPDSGTR

SEQ ID NO: 163 - protein, 14 aa, artificial sequence
  synthetic
CSRSYGSRLAWFAY

SEQ ID NO: 164 - protein, 9 aa, artificial sequence
  synthetic
TGAVTTSNF

SEQ ID NO: 165 - protein, 3 aa, artificial sequence
  synthetic
GTN

SEQ ID NO: 166 - protein, 9 aa, artificial sequence
  synthetic
ALWYTNHFI

SEQ ID NO: 167 - protein, 120 aa, artificial sequence
  synthetic
QVQLQQPGAELVKPGASVKLSCKASGDTFTSYWMHWVKQRPGQGLEWIGMIHPDSGTRNYNEKVMSKATLNVDKSSSTAYLHLSSLTSEDSAVYYCSRSYGSRLAWFAYWGQGTLVTVSA

SEQ ID NO: 168 - protein, 109 aa, artificial sequence
  synthetic
QAVVTQESALTTSPGETVTLTCRSSTGAVTTSNFANWVQEKPDHLFTGLIGGTNNRAPGVPARFSGSLIGDKAALTITGAQTEDEAVYFCALWYTNHFIFGSGTKVTVL

SEQ ID NO: 169 - protein, 8 aa, artificial sequence
  synthetic
DYTFTNYW

SEQ ID NO: 170 - protein, 8 aa, artificial sequence
  synthetic
IHPNSGTT

SEQ ID NO: 171 - protein, 13 aa, artificial sequence
  synthetic
ASHYGNSYSAMDY

SEQ ID NO: 172 - protein, 9 aa, artificial sequence
  synthetic
TGAVTTSNY

SEQ ID NO: 173 - protein, 3 aa, artificial sequence
  synthetic
GTS

SEQ ID NO: 174 - protein, 9 aa, artificial sequence
  synthetic
ALWYSTHYV

SEQ ID NO: 175 - protein, 120 aa, artificial sequence
  synthetic
QVQLQQPGAELVKPGASVKLSCKASDYTFTNYWMHWVKQRPGQGLEWIGMIHPNSGTTSYNEKFKNKATLTVDTSSATTYMHLSSLTSEDSAVYYCASHYGNSYSAMDYWGQGTSVTVSS

SEQ ID NO: 176 - protein, 109 aa, artificial sequence
  synthetic
QAVVTQESALTTSPGGTVILTCRSSTGAVTTSNYANWVQEKPDHLFTGLIGGTSNRAPGVPVRFSGSLIGDKAALTITGAQTEDDAMYFCALWYSTHYVFGGGTKVTVL

SEQ ID NO: 177 - protein, 9 aa, artificial sequence
  synthetic
GYSITSGYY

SEQ ID NO: 178 - protein, 8 aa, artificial sequence
  synthetic
YISYDGTN

SEQ ID NO: 179 - protein, 12 aa, artificial sequence
  synthetic
ARGDSNSYYFDY

SEQ ID NO: 180 - protein, 9 aa, artificial sequence
  synthetic
TGAVTTSNY

SEQ ID NO: 181 - protein, 3 aa, artificial sequence
  synthetic
GTS

SEQ ID NO: 182 - protein, 9 aa, artificial sequence
  synthetic
ALWYRTHYV

SEQ ID NO: 183 - protein, 119 aa, artificial sequence
  synthetic
DVQLQESGPGLVKPSQSLSLTCSVTGYSITSGYYWNWIRQFPGNKLEWMVYISYDGTNNYNPSLKNRISITRDTSKNQFFLKLNSVTTEDTATYYCARGDSNSYYFDYWGQGTTLTVSS

SEQ ID NO: 184 - protein, 109 aa, artificial sequence
  synthetic
QAVVTQESALTTSPGGTVILTCRSSTGAVTTSNYANWVQEKPDHLFTGLIGGTSNRAPGVPVRFSGSLIGDKAALTITGAQTEDDAMYFCALWYRTHYVFGGGTKVTVL

SEQ ID NO: 185 - protein, 9 aa, artificial sequence
  synthetic
GYSITSDYY

SEQ ID NO: 186 - protein, 7 aa, artificial sequence
  synthetic
ISYDGTN

SEQ ID NO: 187 - protein, 12 aa, artificial sequence
  synthetic
ARGKDGSYYFDY

SEQ ID NO: 188 - protein, 9 aa, artificial sequence
  synthetic
TGAVTTSNY

SEQ ID NO: 189 - protein, 3 aa, artificial sequence
  synthetic
GTS

SEQ ID NO: 190 - protein, 9 aa, artificial sequence
  synthetic
ALWYRTHYV

SEQ ID NO: 191 - protein, 119 aa, artificial sequence
  synthetic
DVQLQVSGPGLVKPSQSLSLTCSVAGYSITSDYYWNWIRQFPGNKLEWMGYISYDGTNNYNPSLKNRISITRDTSKGQFFLKFNSVTTEDTATYYCARGKDGSYYFDYWGQGTTLTVSS

SEQ ID NO: 192 - protein, 109 aa, artificial sequence
  synthetic
QAVVTQESALTTSPGGTVILTCRSSTGAVTTSNYANWVQEKPDHLFTGLIGGTSNRAPGVPVRFSGSLIGDKAALTITGAQTEDDAMYFCALWYRTHYVFGGGTMVTVL

SEQ ID NO: 193 - protein, 9 aa, artificial sequence
  synthetic
SGYIFTSNW

SEQ ID NO: 194 - protein, 8 aa, artificial sequence
  synthetic
IHPNSGTT

SEQ ID NO: 195 - protein, 14 aa, artificial sequence
  synthetic
AGPTTTRRFYPMDY

SEQ ID NO: 196 - protein, 9 aa, artificial sequence
  synthetic
TGAVTTSNY

SEQ ID NO: 197 - protein, 3 aa, artificial sequence
  synthetic
GTN

SEQ ID NO: 198 - protein, 9 aa, artificial sequence
  synthetic
ALWYSNHFI

SEQ ID NO: 199 - protein, 121 aa, artificial sequence
  synthetic
QVQLQQPGAELVKPGASMKLSCKASGYIFTSNWMHWVKQRPGQGLEWIGMIHPNSGTTSYNEKFKNKAKLTVDKSSSTAYVQLSSLTSEDSAVYYCAGPTTTRRFYPMDYWGQGTSVTVSS

SEQ ID NO: 200 - protein, 109 aa, artificial sequence
  synthetic
QAVVTQESALTTSPGETVTLTCRSSTGAVTTSNYANWVQEKPDHLFTGLIGGTNNRAPGVPARFSGSLIGDKAALTITGAQTEDEAIYFCALWYSNHFIFGSGTKVTVL

SEQ ID NO: 201 - protein, 11 aa, artificial sequence
  synthetic
DYTFTSHWMHW

SEQ ID NO: 202 - protein, 8 aa, artificial sequence
  synthetic
IHPNSGTT

SEQ ID NO: 203 - protein, 14 aa, artificial sequence
  synthetic
AWGSSNSYFYAMAY

SEQ ID NO: 204 - protein, 9 aa, artificial sequence
  synthetic
TGAVTTSNY

SEQ ID NO: 205 - protein, 3 aa, artificial sequence
  synthetic
GTN

SEQ ID NO: 206 - protein, 9 aa, artificial sequence
  synthetic
ALWFSNHLV

SEQ ID NO: 207 - protein, 121 aa, artificial sequence
  synthetic
QVQLQQPGAELVKPGASVKLSCKASDYTFTSHWMHWVKQRPGQGLEWIGMIHPNSGTTHYNENFKNKATLTVDNSSSTAYMQLSSLTSEDSAVYYCAWGSSNSYFYAMAYWGQGTSVTVSS

SEQ ID NO: 208 - protein, 109 aa, artificial sequence
  synthetic
QAVVTQESALTTSPGETVTLTCRSSTGAVTTSNYANWVQEKPAHLFTGLIGGTNNRAPGVPARFSGSLIGDKAALTITGAQTEDEAIYFCALWFSNHLVFGGGTKLTVL

SEQ ID NO: 209 - protein, 8 aa, artificial sequence
  synthetic
GYTFTSYW

SEQ ID NO: 210 - protein, 8 aa, artificial sequence
  synthetic
IHPNSDTT

SEQ ID NO: 211 - protein, 12 aa, artificial sequence
  synthetic
ARGGLGPHYFVY

SEQ ID NO: 212 - protein, 9 aa, artificial sequence
  synthetic
TGAVTTSNY

SEQ ID NO: 213 - protein, 3 aa, artificial sequence
  synthetic
GTS

SEQ ID NO: 214 - protein, 9 aa, artificial sequence
  synthetic
ALWYSPHYV

SEQ ID NO: 215 - protein, 119 aa, artificial sequence
  synthetic
QVQLQQPGAELVKPGASVKLSCKASGYTFTSYWMHWVKQRPGQGLEWIGMIHPNSDTTDYNEKFKTKATLTVDKSSSTAYMQLSSLTSEDSAVFYCARGGLGPHYFVYWGQGTTLTVSS

SEQ ID NO: 216 - protein, 109 aa, artificial sequence
  synthetic
QAVVTQESALTTSPGGTVILTCRSSTGAVTTSNYASWVQEKPDHLFTGLIGGTSNRAPGVPVRFSGSLIGDKAALTITGAQTEDDAMYFCALWYSPHYVFGGGTKVTVL

SEQ ID NO: 217 - protein, 8 aa, artificial sequence
  synthetic
GYTFTTYW

SEQ ID NO: 218 - protein, 8 aa, artificial sequence
  synthetic
IYPGDGDT

SEQ ID NO: 219 - protein, 15 aa, artificial sequence
  synthetic
ARPYDGYYIDYPMDS

SEQ ID NO: 220 - protein, 5 aa, artificial sequence
  synthetic
SRINY

SEQ ID NO: 221 - protein, 3 aa, artificial sequence
  synthetic
DTS

SEQ ID NO: 222 - protein, 9 aa, artificial sequence
  synthetic
HQRSSYPFT

SEQ ID NO: 223 - protein, 122 aa, artificial sequence
  synthetic
QVQLQQSGAELARPGTSVKLSCKASGYTFTTYWIQWVKQRPGQGLEWIGAIYPGDGDTRYTQKFKGKATLTADKSSSTAYMQLSTLASEDSAVYYCARPYDGYYIDYPMDSWGQGTSVTVSS

SEQ ID NO: 224 - protein, 106 aa, artificial sequence
  synthetic
QIVLTQSPAIMSASPGEKVTMTCSASSRINYMHWYQQKPGTSPIIWIYDTSKLASGVPARFSGSGSGTSYSLTISSMEAEDAATYYCHQRSSYPFTFGSGTKLEIK

SEQ ID NO: 225 - protein, 8 aa, artificial sequence
  synthetic
GYTFTNYI

SEQ ID NO: 226 - protein, 8 aa, artificial sequence
  synthetic
INPYNDGI

SEQ ID NO: 227 - protein, 14 aa, artificial sequence
  synthetic
TRWGNYVFYYTMDY

SEQ ID NO: 228 - protein, 5 aa, artificial sequence
  synthetic
SSVSY

SEQ ID NO: 229 - protein, 3 aa, artificial sequence
  synthetic
ATS

SEQ ID NO: 230 - protein, 10 aa, artificial sequence
  synthetic
QQWSGNPPLT

SEQ ID NO: 231 - protein, 121 aa, artificial sequence
  synthetic
EVQLQQSGPELVKPGASVKMSCKASGYTFTNYIMHWVKQKPGQGLEWIGYINPYNDGIKYNEKFKGKATLTSDKSSSTAYMELSSLTSEDSAVYYCTRWGNYVFYYTMDYWGQGTSVTVSS

SEQ ID NO: 232 - protein, 107 aa, artificial sequence
  synthetic
QIVLSQSPAILSASPGEKVTMTCRASSSVSYMHWYQQKPGSSPKPWIYATSNLASGVPARFSGSGSGTSYSLTISRMEAEDAATYYCQQWSGNPPLTFGAGTKLELK

SEQ ID NO: 233 - protein, 8 aa, artificial sequence
  synthetic
GFTFRSYG

SEQ ID NO: 234 - protein, 8 aa, artificial sequence
  synthetic
ISGGGNYT

SEQ ID NO: 235 - protein, 10 aa, artificial sequence
  synthetic
ARHEGLPLDY

SEQ ID NO: 236 - protein, 6 aa, artificial sequence
  synthetic
ENIYSY

SEQ ID NO: 237 - protein, 3 aa, artificial sequence
  synthetic
NAK

SEQ ID NO: 238 - protein, 10 aa, artificial sequence
  synthetic
QHHYGTPTWT

SEQ ID NO: 239 - protein, 117 aa, artificial sequence
  synthetic
EVQLVESGGDLVKPGGSLKLSCAASGFTFRSYGMSWVRQTPDKRLEWVATISGGGNYTYYPDSVKGRFTISRDNAKNTLYLQMSSLKSEDTAMYYCARHEGLPLDYWGQGTTLTVSS

SEQ ID NO: 240 - protein, 108 aa, artificial sequence
  synthetic
DIQMTQSPASLSASVGETVTITCRASENIYSYLAWYQQKQGKSPQLLVYNAKTLAEGVPSRFSGSGSGTQFSLKINSLQPEDFGNFYCQHHYGTPTWTFGGGTKLEIK

SEQ ID NO: 241 - protein, 8 aa, artificial sequence
  synthetic
GFTFSNYG

SEQ ID NO: 242 - protein, 9 aa, artificial sequence
  synthetic
IGSYGGNTY

SEQ ID NO: 243 - protein, 15 aa, artificial sequence
  synthetic
ARDIYYGNSEAWFAY

SEQ ID NO: 244 - protein, 11 aa, artificial sequence
  synthetic
KSLLHSNGITY

SEQ ID NO: 245 - protein, 3 aa, artificial sequence
  synthetic
QMS

SEQ ID NO: 246 - protein, 9 aa, artificial sequence
  synthetic
AQNLELPWT

SEQ ID NO: 247 - protein, 122 aa, artificial sequence
  synthetic
EVQLVESGGGLVQPGGSLKLSCAASGFTFSNYGLSWVRLTPDKRLEVVATIGSYGGNTYYPDSVKGRFTISRDNAKNTLYLQMSSLKSEDTAIYYCARDIYYGNSEAWFAYWGQGTLVTVSA

SEQ ID NO: 248 - protein, 112 aa, artificial sequence
  synthetic
DIVMTQAAFSNPVTLGTSASISCRSSKSLLHSNGITYLYWYLQKPGQSPQLLIYQMSNLASGVPDRFSSSGSETDFTLRISRVEAEDVGVYYCAQNLELPWTFGGGTKLEIK

SEQ ID NO: 249 - protein, 8 aa, artificial sequence
  synthetic
GYTFTDYA

SEQ ID NO: 250 - protein, 8 aa, artificial sequence
  synthetic
INTNYVNT

SEQ ID NO: 251 - protein, 15 aa, artificial sequence
  synthetic
ARGGKYGNYVFFFAY

SEQ ID NO: 252 - protein, 6 aa, artificial sequence
  synthetic
QDISNY

SEQ ID NO: 253 - protein, 3 aa, artificial sequence
  synthetic
YTS

SEQ ID NO: 254 - protein, 9 aa, artificial sequence
  synthetic
QQGNTLPYT

SEQ ID NO: 255 - protein, 122 aa, artificial sequence
  synthetic
QVQLQQSGAELVRPGVSVKISCKGSGYTFTDYAMHWVKQSHAKSLEWIGVINTNYVNTSYNQKFKGKATMTVDKSSSTAFMELARLTSEDSAIYYCARGGKYGNYVFFFAYWGQGTLVTVSA

SEQ ID NO: 256 - protein, 107 aa, artificial sequence
  synthetic
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPYTFGGGTKLEIK

SEQ ID NO: 257 - protein, 8 aa, artificial sequence
  synthetic
GYIFRNYG

SEQ ID NO: 258 - protein, 10 aa, artificial sequence
  synthetic
INSYTGKPTY

SEQ ID NO: 259 - protein, 12 aa, artificial sequence
  synthetic
ARDDGYWYTMDY

SEQ ID NO: 260 - protein, 11 aa, artificial sequence
  synthetic
QSLVHSNGHTY

SEQ ID NO: 261 - protein, 3 aa, artificial sequence
  synthetic
KVS

SEQ ID NO: 262 - protein, 9 aa, artificial sequence
  synthetic
SQSTHVPWT

SEQ ID NO: 263 - protein, 119 aa, artificial sequence
  synthetic
QIQLVQSGPELKKPGETVKISCKASGYIFRNYGMNWVKQAPGKGLKWMGWINSYTGKPTYVDDFKGRFAFSLETSASTAYLQINNLKNEDMATYFCARDDGYWYTMDYWGQGTSVTVSS

SEQ ID NO: 264 - protein, 112 aa, artificial sequence
  synthetic
DVVMTQTPLSLPVSLGDQASISCRSSQSLVHSNGHTYLHWYLQKPGQSPKLLIYKVSNRFSGVPDRFSGSGSGTDFTLKISRVEAEDLGVYFCSQSTHVPWTFGGGTKLEIK

SEQ ID NO: 265 - protein, 8 aa, artificial sequence
  synthetic
GYTFRNFG

SEQ ID NO: 266 - protein, 8 aa, artificial sequence
  synthetic
INPYTGEP

SEQ ID NO: 267 - protein, 12 aa, artificial sequence
  synthetic
ARDDGYWYTMDY

SEQ ID NO: 268 - protein, 11 aa, artificial sequence
  synthetic
QSLVHSNGITY

SEQ ID NO: 269 - protein, 3 aa, artificial sequence
  synthetic
KVS

SEQ ID NO: 270 - protein, 9 aa, artificial sequence
  synthetic
SQSTHVPWT

SEQ ID NO: 271 - protein, 119 aa, artificial sequence
  synthetic
QIQLVQSGPELKKPGETVKISCKASGYTFRNFGMNWVKQGPGKGLKWMGWINPYTGEPTYVGDFKGRFAFSLETSASTAYLQINNLKNEDMATYFCARDDGYWYTMDYWGQGTSVTVSS

SEQ ID NO: 272 - protein, 112 aa, artificial sequence
  synthetic
DVVMTQTPPSLPVSLGDQASIPCRSSQSLVHSNGITYLHWYLQKPGQSPKLLIYKVSNRFSGVPDRFSGSGSGTDFTLKISRVEAEDLGVYFCSQSTHVPWTFGGGTKLEIK

SEQ ID NO: 273 - protein, 8 aa, artificial sequence
  synthetic
GYTFRNYG

SEQ ID NO: 274 - protein, 8 aa, artificial sequence
  synthetic
INPYTGEP

SEQ ID NO: 275 - protein, 12 aa, artificial sequence
  synthetic
ARDDGYWYTMDY

SEQ ID NO: 276 - protein, 11 aa, artificial sequence
  synthetic
QSLVHSNGITY

SEQ ID NO: 277 - protein, 3 aa, artificial sequence
  synthetic
KVS

SEQ ID NO: 278 - protein, 9 aa, artificial sequence
  synthetic
SQSTHVPWT

SEQ ID NO: 279 - protein, 119 aa, artificial sequence
  synthetic
QIQLVQSGPELKKPGETVKISCKASGYTFRNYGMNWVKQGPGKDLKWMGWINPYTGEPTYIGDFKGRFAFSLETSANSAYLQINNLNNEDMATYFCARDDGYWYTMDYWGQGTSVTVSS

SEQ ID NO: 280 - protein, 112 aa, artificial sequence; identical to SEQ ID NO: 272
  synthetic

SEQ ID NO: 281 - protein, 8 aa, artificial sequence
  synthetic
GYTFTEYA

SEQ ID NO: 282 - protein, 8 aa, artificial sequence
  synthetic
INPNIGGT

SEQ ID NO: 283 - protein, 13 aa, artificial sequence
  synthetic
ARFLVPSTEGFTY

SEQ ID NO: 284 - protein, 5 aa, artificial sequence
  synthetic
SSVSY

SEQ ID NO: 285 - protein, 3 aa, artificial sequence
  synthetic
DTS

SEQ ID NO: 286 - protein, 9 aa, artificial sequence
  synthetic
QQWSSNPLT

SEQ ID NO: 287 - protein, 120 aa, artificial sequence
  synthetic
EVQLQQSGPELVKPGASVKISCKTSGYTFTEYAMHWVKQSHGKILEWIGGINPNIGGTTYNQKFKDKATLTVDKSSSTASMELRSLTSEDSAVYYCARFLVPSTEGFTYWGQGTLVTVSA

SEQ ID NO: 288 - protein, 106 aa, artificial sequence
  synthetic
QIVLTQSPAIMSASPGEKVTMTCSASSSVSYMHWYQQKSGTSPKRWIYDTSRVASGVPARFSGSGSGTSYSLTISSMEAEDAATYYCQQWSSNPLTFGAGTKLELK

SEQ ID NO: 289 - protein, 8 aa, artificial sequence
  synthetic
GYTFTDYV

SEQ ID NO: 290 - protein, 8 aa, artificial sequence
  synthetic
IYPRSGNT

SEQ ID NO: 291 - protein, 11 aa, artificial sequence
  synthetic
ARFDIAYSFDY

SEQ ID NO: 292 - protein, 11 aa, artificial sequence
  synthetic
QSLVHSNGVTY

SEQ ID NO: 293 - protein, 3 aa, artificial sequence
  synthetic
KVS

SEQ ID NO: 294 - protein, 9 aa, artificial sequence
  synthetic
SQGTHVPWT

SEQ ID NO: 295 - protein, 118 aa, artificial sequence
  synthetic
QVQLQQSGPELVKPGASVQMSCKASGYTFTDYVISWVNQRTGQGLEWIGEIYPRSGNTYYNEKFKGKATLTADKSSNTAYMQLSSLTSEDSAVYFCARFDIAYSFDYWGQGTTLTVSS

SEQ ID NO: 296 - protein, 112 aa, artificial sequence
  synthetic
DVVMTQTPLSLPVSLGDQASISCRSTQSLVHSNGVTYLHWYLQKPGQSPKLLIYKVSNRFSGVPDRFSGSGSGTDFTLKISRVEAEDLGVYFCSQGTHVPWTFGGGTKLEIK

SEQ ID NO: 297 - protein, 8 aa, artificial sequence
  synthetic
GYSFTDYI

SEQ ID NO: 298 - protein, 8 aa, artificial sequence
  synthetic
INPYYNST

SEQ ID NO: 299 - protein, 11 aa, artificial sequence
  synthetic
ARDSFDWYFGV

SEQ ID NO: 300 - protein, 11 aa, artificial sequence
  synthetic
QSLVHSNGNTY

SEQ ID NO: 301 - protein, 3 aa, artificial sequence
  synthetic
KVS

SEQ ID NO: 302 - protein, 10 aa, artificial sequence
  synthetic
SQSTHVPPLT

SEQ ID NO: 303 - protein, 118 aa, artificial sequence
  synthetic
EIQLQQTGPELVKPGASVKISCKASGYSFTDYIILWVKQSHGMSLEWIGNINPYYNSTYYNLKFKGKATLTVDKSSSTAYMQLDSLTSEDSAVYYCARDSFDWYFGVWGAGTTVTVSS

SEQ ID NO: 304 - protein, 113 aa, artificial sequence
  synthetic
DVVMTQTPLSLPVSLGDQASISCRSSQSLVHSNGNTYLHWYLQKPGQSPKLLIYKVSNRFSGVPDRFSGSGSGTDFTLKISRVETEDLGIYFCSQSTHVPPLTFGAGTKLELK

SEQ ID NO: 305 - protein, 8 aa, artificial sequence
  synthetic
GFTFITYA

SEQ ID NO: 306 - protein, 8 aa, artificial sequence
  synthetic
ISGGGIDT

SEQ ID NO: 307 - protein, 13 aa, artificial sequence
  synthetic
TRQGSQGWGAMDY

SEQ ID NO: 308 - protein, 5 aa, artificial sequence
  synthetic
SRVSF

SEQ ID NO: 309 - protein, 3 aa, artificial sequence
  synthetic
ETS

SEQ ID NO: 310 - protein, 9 aa, artificial sequence
  synthetic
QQWSSNPLT

SEQ ID NO: 311 - protein, 120 aa, artificial sequence
  synthetic
EVMLVESGGGLVKPGGSLKFSCAASGFTFITYAMSWVRQTPEKRLEWVATISGGGIDTYYPDSVKGRFTISRDNAKNTLYLQVNSLRSEDTAMYYCTRQGSQGWGAMDYWGQGTSVTVSS

SEQ ID NO: 312 - protein, 106 aa, artificial sequence
  synthetic
QIVLTQSPPIMSASPGERVTMTCRASSRVSFMHWYQQKSGTSPRRWIYETSKLASGVPDRFSGSVSGTSYSLTISSMEAEDAATYYCQQWSSNPLTFGAGTKLELK

SEQ ID NO: 313 - protein, 8 aa, artificial sequence
  synthetic
GYTFTDYT

SEQ ID NO: 314 - protein, 8 aa, artificial sequence
  synthetic
ISTYYGNT

SEQ ID NO: 315 - protein, 18 aa, artificial sequence
  synthetic
ARASYGSGYGPYYWYFDV

SEQ ID NO: 316 - protein, 6 aa, artificial sequence
  synthetic
EKIYSY

SEQ ID NO: 317 - protein, 3 aa, artificial sequence
  synthetic
NAN

SEQ ID NO: 318 - protein, 9 aa, artificial sequence
  synthetic
QHHYGSPVT

SEQ ID NO: 319 - protein, 125 aa, artificial sequence
  synthetic
QVQLQQSGPELVRPGVSVKISCKGSGYTFTDYTMHWVKESHAQSLEWIGVISTYYGNTNYNQNFKGKATLTVDKSSSTAYMELAGLTSEDSAIYYCARASYGSGYGPYYWYFDVWGAGTTVTVSS

SEQ ID NO: 320 - protein, 107 aa, artificial sequence
  synthetic
DIQMTQSPASLFASVGETVTITCRASEKIYSYLAWYFQKQGKSPQFLVYNANALAEGVPSRFSGSGSGTQFSLKIKSLQPEDFGTYYCQHHYGSPVTFGAGSKLELK

SEQ ID NO: 321 - protein, 8 aa, artificial sequence
  synthetic
GYTFTDYT

SEQ ID NO: 322 - protein, 8 aa, artificial sequence
  synthetic
ISTYYGNT

SEQ ID NO: 323 - protein, 18 aa, artificial sequence
  synthetic
ARASYGSGYGPYYWYFDV

SEQ ID NO: 324 - protein, 6 aa, artificial sequence
  synthetic
EKIYSY

SEQ ID NO: 325 - protein, 3 aa, artificial sequence
  synthetic
NAN

SEQ ID NO: 326 - protein, 9 aa, artificial sequence
  synthetic
QHHYGTPVT

SEQ ID NO: 327 - protein, 125 aa, artificial sequence
  synthetic
QVQLQQSGPELVRPGVSVKISCKGSGYTFTDYTMHWVKESHAKSLEWIGVISTYYGNTNYNQKFKGKATLTVDKSSSTAYMELAGLTSEDSAIYYCARASYGSGYGPYYWYFDVWGAGTTVTVSS

SEQ ID NO: 328 - protein, 107 aa, artificial sequence; identical to SEQ ID NO: 128
  synthetic

SEQ ID NO: 329 - protein, 8 aa, artificial sequence
  synthetic
GYTFTNYW

SEQ ID NO: 330 - protein, 8 aa, artificial sequence
  synthetic
IHPNSGTT

SEQ ID NO: 331 - protein, 13 aa, artificial sequence
  synthetic
AGYYGSSFSWFTY

SEQ ID NO: 332 - protein, 9 aa, artificial sequence
  synthetic
TGAVTTSNF

SEQ ID NO: 333 - protein, 3 aa, artificial sequence
  synthetic
GTS

SEQ ID NO: 334 - protein, 9 aa, artificial sequence
  synthetic
ALWYSTHYV

SEQ ID NO: 335 - protein, 120 aa, artificial sequence
  synthetic
QVHLQQPGAELVKPGASVKLSCKASGYTFTNYWMHWVKRRPGQGLEWIGMIHPNSGTTSFNEKFKSKATLTVDKSSSTAYMQLSSLTSEDSAVYYCAGYYGSSFSWFTYWGQGTLVTVSA

SEQ ID NO: 336 - protein, 109 aa, artificial sequence
  synthetic
QAVVTQESALTTSPGGTVILTCRSSTGAVTTSNFANWVQEKPDHLFTGLIGGTSNRAPGVPVRFSGSLIGDKAALTITGAQTEDDAMYFCALWYSTHYVFGGGTKVTVL

SEQ ID NO: 337 - protein, 8 aa, artificial sequence
  synthetic
GYIFRNYG

SEQ ID NO: 338 - protein, 8 aa, artificial sequence
  synthetic
INSYTGKP

SEQ ID NO: 339 - protein, 12 aa, artificial sequence
  synthetic
ARDDGYWYTMDY

SEQ ID NO: 340 - protein, 11 aa, artificial sequence
  synthetic
QSLVHSNGHTY

SEQ ID NO: 341 - protein, 3 aa, artificial sequence
  synthetic
KVS

SEQ ID NO: 342 - protein, 9 aa, artificial sequence
  synthetic
SQSTHVPWT

SEQ ID NO: 343 - protein, 119 aa, artificial sequence; identical to SEQ ID NO: 263
  synthetic

SEQ ID NO: 344 - protein, 112 aa, artificial sequence; identical to SEQ ID NO: 264
  synthetic

SEQ ID NO: 345 - protein, 5 aa, artificial sequence
  synthetic
DDDDK

SEQ ID NO: 346 - protein, 449 aa, artificial sequence
  synthetic
LEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEAHHHHHHDDDDK

SEQ ID NO: 347 - protein, 120 aa, artificial sequence
  synthetic
EVQLVESGGGLVQPGGSLRLSCATSGFTLSDYYMSWVRQPPGKAPEWLGFMRNKANAYTTEYSASVRGRFTISRDTSQSIIYLQMNTLRAEDGATYYCVRDREGRPFAYWGQGTLVTVSA

SEQ ID NO: 348 - protein, 113 aa, artificial sequence
  synthetic
DIVMTQSPSSLTVTAGEKVTMSCKSSQSLLNAINQKNFLTWYQQKPGQPPKLLIYWASTRESGVPDRFTGSGSGTDFTLTISSVQAEDLAIYYCQNDYTYPLTFGTGTKLELK

SEQ ID NO: 349 - protein, 119 aa, artificial sequence; identical to SEQ ID NO: 47
  synthetic

SEQ ID NO: 350 - protein, 113 aa, artificial sequence
  synthetic
DIVMTQSPSSLAMSVGQKVTLSCKSSQSLLQSSTQKNYLAWYQQKPGQSPKLLVYFASTRDSGVPDRFIGSGSGTEFTLTISSVQAEDLADYFCQQHYIIPFTFGAGTKLEIK

SEQ ID NO: 351 - protein, 120 aa, artificial sequence
  synthetic
EVQLVESGGGLVQPGGSLRLSCATSGFTLSDYYMSWVRQAPGKGLEWVSFMRNKANGYTTEYSASVRGRFTISRDTSKSTIYLQMNSLRAEDTAVYYCVRDRDGRPFAYWGQGTLVTVSS

SEQ ID NO: 352 - protein, 113 aa, artificial sequence
  synthetic
DIVMTQSPDSLAVSLGERATINCKSSQSLLNSINQKNFLTWYQQKPGQPPKLLIYWASTRESGVPDRFSGSGSGTDFTLTISSLQAEDLAVYYCQNDYTYPLTFGQGTKLEIK

SEQ ID NO: 353 - protein, 120 aa, artificial sequence
  synthetic
EVQLVESGGGLVQPGGSLRLSCATSGFTLSDYYMSWVRQAPGKGLEWVGFMRNKANGYTTEYSASVRGRFTISRDTSKSSIYLQMNSLKTEDTAVYYCVRDRDGRPFAYWGQGTLVTVSS

SEQ ID NO: 354 - protein, 113 aa, artificial sequence
  synthetic
DIQMTQSPSSLSASVGDRVTITCKSSQSLLNSINQKNFLTWYQQKPGKAPKLLIYWASTRESGVPSRFSGSGSGTDFTLTISSLQPEDLATYYCQNDYTYPLTFGQGTKLEIK

SEQ ID NO: 355 - protein, 120 aa, artificial sequence
  synthetic
EVQLVESGGGLIQPGGSLRLSCATSGFTLSDYYMSWVRQPPGKGLEWVSFMRNKANGYTTEYSASVRGRFTISRDTSKSTIYLQMNSLRAEDTAVYYCVRDRDGRPFAYWGQGTLVTVSS

SEQ ID NO: 356 - protein, 113 aa, artificial sequence
  synthetic
DIVMTQSPLSLPVTPGEPASISCKSSQSLLNSINQKNFLTWYLQKPGQSPQLLIYWASTRESGVPDRFSGSGSGTDFTLKISRVEAEDLGVYYCQNDYTYPLTFGQGTKLEIK

SEQ ID NO: 357 - protein, 120 aa, artificial sequence
  synthetic
EVQLVESGGGLVQPGRSLRLSCTTSGFTLSDYYMSWVRQAPGKGLEWVGFMRNKANGYTTEYSASVRGRFTISRDTSKSIIYLQMNSLKTEDTAVYYCVRDRDGRPFAYWGQGTLVTVSS

SEQ ID NO: 358 - protein, 120 aa, artificial sequence
  synthetic
EVQLVESGGGLVQPGGSLRLSCATSGFTLSDYYMSWVRQAPGKGLEWVSFMRNKANAYTTEYSASVRGRFTISRDTSKSTIYLQMNSLRAEDTAVYYCVRDREGRPFAYWGQGTLVTVSS

SEQ ID NO: 359 - protein, 113 aa, artificial sequence
  synthetic
DIVMTQSPDSLAVSLGERATINCKSSQSLLNAINQKNFLTWYQQKPGQPPKLLIYWASTRESGVPDRFSGSGSGTDFTLTISSLQAEDLAVYYCQNDYTYPLTFGQGTKLEIK

SEQ ID NO: 360 - protein, 120 aa, artificial sequence
  synthetic
EVQLVESGGGLVQPGGSLRLSCATSGFTLSDYYMSWVRQAPGKGLEWVGFMRNKANAYTTEYSASVRGRFTISRDTSKSSIYLQMNSLKTEDTAVYYCVRDREGRPFAYWGQGTLVTVSS

SEQ ID NO: 361 - protein, 113 aa, artificial sequence
  synthetic
DIQMTQSPSSLSASVGDRVTITCKSSQSLLNAINQKNFLTWYQQKPGKAPKLLIYWASTRESGVPSRFSGSGSGTDFTLTISSLQPEDLATYYCQNDYTYPLTFGQGTKLEIK

SEQ ID NO: 362 - protein, 120 aa, artificial sequence
  synthetic
EVQLVESGGGLIQPGGSLRLSCATSGFTLSDYYMSWVRQPPGKGLEWVSFMRNKANAYTTEYSASVRGRFTISRDTSKSTIYLQMNSLRAEDTAVYYCVRDREGRPFAYWGQGTLVTVSS

SEQ ID NO: 363 - protein, 113 aa, artificial sequence
  synthetic
DIVMTQSPLSLPVTPGEPASISCKSSQSLLNAINQKNFLTWYLQKPGQSPQLLIYWASTRESGVPDRFSGSGSGTDFTLKISRVEAEDLGVYYCQNDYTYPLTFGQGTKLEIK

SEQ ID NO: 364 - protein, 120 aa, artificial sequence
  synthetic
EVQLVESGGGLVQPGRSLRLSCTTSGFTLSDYYMSWVRQAPGKGLEWVGFMRNKANAYTTEYSASVRGRFTISRDTSKSIIYLQMNSLKTEDTAVYYCVRDREGRPFAYWGQGTLVTVSS

SEQ ID NO: 365 - protein, 119 aa, artificial sequence
  synthetic
QVQLQESGPGLVKPSQTLSLTCTVSGYSITSDYAWNWIRQHPGKGLEWIGYISHSGSTSYNPSLKSRVTISRDTSKNQFSLKLSSVTAADTAVYYCARSLGRRWYFDVWGQGTTVTVSS

SEQ ID NO: 366 - protein, 113 aa, artificial sequence
  synthetic
DIVMTQSPLSLPVTPGEPASISCKSSQSLLNSSTQKNYLAWYLQKPGQSPQLLIYFASTRDSGVPDRFSGSGSGTDFTLKISRVEAEDLGVYFCQQHYIIPFTFGQGTKLEIK

SEQ ID NO: 367 - protein, 119 aa, artificial sequence
  synthetic
QVQLQESGPGLVKPSETLSLTCTVSGYSITSDYAWNWIRQPPGKGLEWIGYISHSGSTSYNPSLKSRVTISRDTSKNQFSLKLSSVTAADTAVYYCARSLGRRWYFDVWGQGTTVTVSS

SEQ ID NO: 368 - protein, 113 aa, artificial sequence
  synthetic
DIQMTQSPSSLSASVGDRVTITCKSSQSLLNSSTQKNYLAWYQQKPGKAPKLLIYFASTRDSGVPSRFSGSGSGTDFTLTISSLQPEDLATYFCQQHYIIPFTFGQGTKLEIK

SEQ ID NO: 369 - protein, 113 aa, artificial sequence
  synthetic
DIVMTQSPDSLAVSLGERATINCKSSQSLLNSSTQKNYLAWYQQKPGQPPKLLIYFASTRDSGVPDRFSGSGSGTDFTLTISSLQAEDLAVYFCQQHYIIPFTFGQGTKLEIK

SEQ ID NO: 370 - protein, 119 aa, artificial sequence
  synthetic
QVQLQESGPGLVKPSQTLSLTCAVYGYSITSDYAWNWIRQPPGKGLEWIGYISHSGSTSYNPSLKSRVTISRDTSKNQFSLKLSSVTAADTAVYYCARSLGRRWYFDVWGQGTTVTVSS

SEQ ID NO: 371 - protein, 113 aa, artificial sequence
  synthetic
DIVMTQSPLSLPVTPGEPASISCKSSQSLLQSSTQKNYLAWYLQKPGQSPQLLIYFASTRDSGVPDRFSGSGSGTDFTLKISRVEAEDLGVYFCQQHYIIPFTFGQGTKLEIK

SEQ ID NO: 372 - protein, 113 aa, artificial sequence
  synthetic
DIQMTQSPSSLSASVGDRVTITCKSSQSLLQSSTQKNYLAWYQQKPGKAPKLLIYFASTRDSGVPSRFSGSGSGTDFTLTISSLQPEDLATYFCQQHYIIPFTFGQGTKLEIK

SEQ ID NO: 373 - protein, 113 aa, artificial sequence
  synthetic
DIVMTQSPDSLAVSLGERATINCKSSQSLLQSSTQKNYLAWYQQKPGQPPKLLIYFASTRDSGVPDRFSGSGSGTDFTLTISSLQAEDLAVYFCQQHYIIPFTFGQGTKLEIK

SEQ ID NO: 374 - protein, 10 aa, artificial sequence
  synthetic
MRNKANAYTT

SEQ ID NO: 375 - protein, 11 aa, artificial sequence
  synthetic
VRDREGRPFAY

SEQ ID NO: 376 - protein, 12 aa, artificial sequence
  synthetic
QSLLNAINQKNF

SEQ ID NO: 377 - protein, 12 aa, artificial sequence
  synthetic
QSLLQSSTQKNY

SEQ ID NO: 378 - protein, 120 aa, artificial sequence
  synthetic
  misc_feature (12)..(12): Xaa can be Val or Ile
  misc_feature (16)..(16): Xaa can be Gly or Arg
  misc_feature (23)..(23): Xaa can be Ala or Thr
  misc_feature (40)..(40): Xaa can be Ala or Pro
  misc_feature (49)..(49): Xaa can be Gly or Ser
  misc_feature (57)..(57): Xaa can be Ala or Gly
  misc_feature (80)..(80): Xaa can be Ile or Thr
  misc_feature (89)..(89): Xaa can be Arg or Lys
  misc_feature (90)..(90): Xaa can be Ala or Thr
  misc_feature (103)..(103): Xaa can be Asp or Glu
EVQLVESGGGLXQPGXSLRLSCXTSGFTLSDYYMSWVRQXPGKGLEWVXFMRNKANXYTTEYSASVRGRFTISRDTSKSXIYLQMNSLXXEDTAVYYCVRDRXGRPFAYWGQGTLVTVSS

SEQ ID NO: 379 - protein, 113 aa, artificial sequence
  synthetic
  misc_feature (3)..(3): Xaa can be Val or Gln
  misc_feature (9)..(9): Xaa can be Asp, Leu or Ser
  misc_feature (12)..(12): Xaa can be Ala, Ser or Pro
  misc_feature (13)..(13): Xaa can be Ala or Val
  misc_feature (14)..(14): Xaa can be Ser or Thr
  misc_feature (15)..(15): Xaa can be Leu, Val or Pro
  misc_feature (17)..(17): Xaa can be Asp or Glu
  misc_feature (18)..(18): Xaa can be Arg or Pro
  misc_feature (19)..(19): Xaa can be Ala or Val
  misc_feature (20)..(20): Xaa can be Ser or Thr
  misc_feature (22)..(22): Xaa can be Asn, Thr or Ser
  misc_feature (32)..(32): Xaa can be Ala or Ser
  misc_feature (43)..(43): Xaa can be Gln or Leu
  misc_feature (48)..(48): Xaa can be Gln or Lys
  misc_feature (49)..(49): Xaa can be Ala, Pro or Ser
  misc_feature (51)..(51): Xaa can be Lys or Gln
  misc_feature (66)..(66): Xaa can be Ser or Asp
  misc_feature (80)..(80): Xaa can be Lys or Thr
  misc_feature (83)..(83): Xaa can be Arg or Ser
  misc_feature (84)..(84): Xaa can be Leu or Val
  misc_feature (85)..(85): Xaa can be Gln or Glu
  misc_feature (86)..(86): Xaa can be Ala or Pro
  misc_feature (90)..(90): Xaa can be Ala or Gly
  misc_feature (91)..(91): Xaa can be Thr or Val
DIXMTQSPXSLXXXXGXXXXIXCKSSQSLLNXINQKNFLTWYXQKPGXXPXLLIYWASTRESGVPXRFSGSGSGTDFTLXISXXXXEDLXXYYCQNDYTYPLTFGQGTKLEIK

SEQ ID NO: 380 - protein, 119 aa, artificial sequence
  synthetic
  misc_feature (16)..(16): Xaa can be Glu or Gln
  misc_feature (23)..(23): Xaa can be Ala or Thr
  misc_feature (25)..(25): Xaa can be Ser or Tyr
  misc_feature (41)..(41): Xaa can be His or Pro
QVQLQESGPGLVKPSXTLSLTCXVXGYSITSDYAWNWIRQXPGKGLEWIGYISHSGSTSYNPSLKSRVTISRDTSKNQFSLKLSSVTAADTAVYYCARSLGRRWYFDVWGQGTTVTVSS

SEQ ID NO: 381 - protein, 113 aa, artificial sequence
  synthetic
  misc_feature (3)..(3): Xaa can be Val or Gln
  misc_feature (9)..(9): Xaa can be Asp, Leu or Ser
  misc_feature (12)..(12): Xaa can be Ala, Ser or Pro
  misc_feature (13)..(13): Xaa can be Ala or Val
  misc_feature (14)..(14): Xaa can be Ser or Thr
  misc_feature (15)..(15): Xaa can be Leu, Val or Pro
  misc_feature (17)..(17): Xaa can be Asp or Glu
  misc_feature (18)..(18): Xaa can be Arg or Pro
  misc_feature (19)..(19): Xaa can be Ala or Val
  misc_feature (20)..(20): Xaa can be Ser or Thr
  misc_feature (22)..(22): Xaa can be Asn, Thr or Ser
  misc_feature (31)..(31): Xaa can be Asn or Gln
  misc_feature (43)..(43): Xaa can be Gln or Leu
  misc_feature (48)..(48): Xaa can be Gln or Lys
  misc_feature (49)..(49): Xaa can be Ala, Pro or Ser
  misc_feature (51)..(51): Xaa can be Lys or Gln
  misc_feature (66)..(66): Xaa can be Ser or Asp
  misc_feature (80)..(80): Xaa can be Lys or Thr
  misc_feature (83)..(83): Xaa can be Arg or Ser
  misc_feature (84)..(84): Xaa can be Leu or Val
  misc_feature (85)..(85): Xaa can be Gln or Glu
  misc_feature (86)..(86): Xaa can be Ala or Pro
  misc_feature (90)..(90): Xaa can be Ala or Gly
  misc_feature (91)..(91): Xaa can be Thr or Val
DIXMTQSPXSLXXXXGXXXXIXCKSSQSLLXSSTQKNYLAWYXQKPGXXPXLLIYFASTRDSGVPXRFSGSGSGTDFTLXISXXXXEDLXXYFCQQHYIIPFTFGQGTKLEIK

(19) United States
(12) Patent Application Publication
(10) Pub. No.: US 2024/0400689 A1
(43) Pub. Date: Dec. 5, 2024

(54) NOVEL ANTI-CD276 ANTIBODIES AND THE USES THEREOF

(71) Applicant: ANTENGENE BIOLOGICS LIMITED, Shanghai (CN)

(72) Inventors: Hui YUWEN, Shanghai (CN); Bing HOU, Shanghai (CN); Tenteng LI, Shanghai (CN); Peng CHEN, Shanghai (CN); Min DENG, Shanghai (CN); Bo SHAN, Shanghai (CN); Jay MEI, Shanghai (CN)

(21) Appl. No.: 18/559,795

(22) PCT Filed: May 11, 2022
(86) PCT No.: PCT/CN2022/092138

§ 371 Date: Nov. 9, 2023

(30) Foreign Application Priority Data
May 11, 2021 (WO) PCT/CN2021/093157
Apr. 27, 2022 (WO) PCT/CN2022/089645

(51) Int. Cl. C07K 16/28; A61K 39/00; A61P 35/00

(52) U.S. Cl. CPC C07K 16/2827; A61P 35/00; A61K 2039/505; C07K 2317/24; C07K 2317/732; C07K 2317/734; C07K 2317/77; C07K 2317/92

(57) ABSTRACT

Provided are anti-CD276 antibodies, and anti-CD276/PD-L1 bispecific antibody molecules, isolated polynucleotides encoding the same, pharmaceutical compositions comprising the same, and the uses thereof.

### FIELD OF THE INVENTION

The present disclosure generally relates to novel anti-CD276 antibodies.

### BACKGROUND

B7-H3 (CD276, UniProt IDs for human amino acid sequence: Q5ZPR3 and mouse amino acid sequence: Q8VE98) is an important newly found immune checkpoint member of the B7 and CD28 families, which is a type I transmembrane costimulatory molecule, existing in two isoforms determined by its extracellular domain. In mice, the extracellular domain consists of a single pair of immunoglobulin variable (IgV)-like and immunoglobulin constant (IgC)-like domains, whereas in humans it consists of one pair (21 g-B7-H3) or two identical pairs (4Ig-B7-H3) due to exon duplication. B7-H3 mRNA is widely distributed in most tissues; in contrast, B7-H3 protein has a very limited expression on normal tissues because of its post-transcriptional regulation by microRNAs. However, B7-H3 protein is expressed at high frequency on many different cancer types (60% of all cancers) (“B7-H3: an attractive target for antibody-based immunotherapy”. Clinical Cancer Research: clincanres.2584.2020).

The function of B7-H3 has been controversial. It was classified as either a costimulatory molecule for T cell activation that inhibits tumor antigen-specific immune responses, or the non-immunological role such as promoting migration, tumor growth, invasion, metastasis, malignant stage, recurrence rate, angiogenesis, chemoresistance, epithelial-to-mesenchymal transition, and affecting tumor cell metabolism. The receptor for B7-H3 has been reported to be triggering receptor expressed on myeloid cell (TREM)-like transcript 2 (TLT-2, or TREML2), which binds B7-H3 and costimulates activation of CD8 T cells in particular. B7-H3 is also reported an inhibitor for NK cells and osteoblastic cells by ligating unknown receptor(s). (The contrasting role of B7-H3, PNAS Jul. 29, 2008, 105 (30) 10277-10278). Treatment with monoclonal antibodies to block B7-H3 are the main immunotherapeutic strategies for cancer treatment.

Based on the clinical success of inhibitory immune checkpoint blockade (CTLA-4, PD-1, and PD-L1), mAbs against CD276 appear to be a promising therapeutic strategy worthy of development. Due to its selective expression on solid tumors, several groups have generated anti-CD276 antibodies, such as enoblituzumab (MGA271), omburtamab, MGD009, MGC018, DS-7300a, and CAR T cells (“B7-H3: an attractive target for antibody-based immunotherapy”. Clinical Cancer Research: clincanres.2584.2020), and observed tumor growth suppression in vitro and in vivo. CD276 is also reported to be expressed in hematological tumor cells (see Wei Zhang et al., B7 Family Members in Lymphoma: Promising Novel Targets for Tumor Immunotherapy? Front. Oncol., 31 Mar. 2021), indicating that CD276 can also be a potential target for treating hematological cancers.

Despite of the development of therapeutics targeting the CD276, there is a significant need for novel anti-CD276 antibodies.

### BRIEF SUMMARY OF THE INVENTION

Throughout the present disclosure, the articles “a,” “an,” and “the” are used herein to refer to one or to more than one (i.e., to at least one) of the grammatical object of the article. By way of example, “an antibody” means one antibody or more than one antibody.

The present disclosure provides novel anti-CD276 antibody molecules, amino acid and nucleotide sequences thereof, and uses thereof.

In one aspect, the present disclosure provides an isolated antibody or an antigen-binding fragment thereof, comprising 1, 2, or 3 heavy chain complementarity determining region (CDR) sequences selected from the group consisting of: SEQ ID NOs: 1-3, 9-11, 17-19, 25-27, 33-35, 41-43, 49-51, 57-59, 65-67, 73-75, 81-83, 89-91, 97-99, 105-107, 113-115, 121-123, 129-131, 137-139, 145-147, 153-155, 161-163, 169-171, 177-179, 185-187, 193-195, 201-203, 209-211, 217-219, 225-227, 233-235, 241-243, 249-251, 257-259, 265-267, 273-275, 281-283, 289-291, 297-299, 305-307, 313-315, 321-323, 329-331, 337-339 and 374-375, and/or 1, 2, or 3 light chain CDR sequences selected from the group consisting of: SEQ ID NOs: 4-6, 12-14, 20-22, 28-30, 36-38, 44-46, 52-54, 60-62, 68-70, 76-78, 84-86, 92-94, 100-102, 108-110, 116-118, 124-126, 132-134, 140-142, 148-150, 156-158, 164-166, 172-174, 180-181, 188-190, 196-198, 204-206, 212-214, 220-222, 228-230, 236-238, 244-246, 252-254, 260-262, 268-270, 276-278, 284-286, 292-294, 300-302, 308-310, 316-318, 324-326, 332-334, 340-342 and 376-377.

In some embodiments, the antibody or antigen-binding fragment thereof provided herein comprises a heavy chain variable region selected from the group consisting of:
a) a heavy chain variable region comprising 1, 2, or 3 CDR sequences selected from SEQ ID NOs: 1-3;
b) a heavy chain variable region comprising 1, 2, or 3 CDR sequences selected from SEQ ID NOs: 9-11;
c) a heavy chain variable region comprising 1, 2, or 3 CDR sequences selected from SEQ ID NOs: 17-19;
d) a heavy chain variable region comprising 1, 2, or 3 CDR sequences selected from SEQ ID NOs: 25-27;
e) a heavy chain variable region comprising 1, 2, or 3 CDR sequences selected from SEQ ID NOs: 33-35;
f) a heavy chain variable region comprising 1, 2, or 3 CDR sequences selected from SEQ ID NOs: 41-43;
g) a heavy chain variable region comprising 1, 2, or 3 CDR sequences selected from SEQ ID NOs: 49-51;
h) a heavy chain variable region comprising 1, 2, or 3 CDR sequences selected from SEQ ID NOs: 57-59;
i) a heavy chain variable region comprising 1, 2, or 3 CDR sequences selected from SEQ ID NOs: 65-67;
j) a heavy chain variable region comprising 1, 2, or 3 CDR sequences selected from SEQ ID NOs: 73-75;
k) a heavy chain variable region comprising 1, 2, or 3 CDR sequences selected from SEQ ID NOs: 81-83;
l) a heavy chain variable region comprising 1, 2, or 3 CDR sequences selected from SEQ ID NOs: 89-91;
m) a heavy chain variable region comprising 1, 2, or 3 CDR sequences selected from SEQ ID NOs: 97-99;
n) a heavy chain variable region comprising 1, 2, or 3 CDR sequences selected from SEQ ID NOs: 105-107;
o) a heavy chain variable region comprising 1, 2, or 3 CDR sequences selected from SEQ ID NOs: 113-115;
p) a heavy chain variable region comprising 1, 2, or 3 CDR sequences selected from SEQ ID NOs: 121-123;
q) a heavy chain variable region comprising 1, 2, or 3 CDR sequences selected from SEQ ID NOs: 129-131;
r) a heavy chain variable region comprising 1, 2, or 3 CDR sequences selected from SEQ ID NOs: 137-139;
s) a heavy chain variable region comprising 1, 2, or 3 CDR sequences selected from SEQ ID NOs: 145-147;
t) a heavy chain variable region comprising 1, 2, or 3 CDR sequences selected from SEQ ID NOs: 153-155;
u) a heavy chain variable region comprising 1, 2, or 3 CDR sequences selected from SEQ ID NOs: 161-163;
v) a heavy chain variable region comprising 1, 2, or 3 CDR sequences selected from SEQ ID NOs: 169-171;
w) a heavy chain variable region comprising 1, 2, or 3 CDR sequences selected from SEQ ID NOs: 177-179;
x) a heavy chain variable region comprising 1, 2, or 3 CDR sequences selected from SEQ ID NOs: 185-187;
y) a heavy chain variable region comprising 1, 2, or 3 CDR sequences selected from SEQ ID NOs: 193-195;
z) a heavy chain variable region comprising 1, 2, or 3 CDR sequences selected from SEQ ID NOs: 201-203;
aa) a heavy chain variable region comprising 1, 2, or 3 CDR sequences selected from SEQ ID NOs: 209-211;
bb) a heavy chain variable region comprising 1, 2, or 3 CDR sequences selected from SEQ ID NOs: 217-219;
cc) a heavy chain variable region comprising 1, 2, or 3 CDR sequences selected from SEQ ID NOs: 225-227;
dd) a heavy chain variable region comprising 1, 2, or 3 CDR sequences selected from SEQ ID NOs: 233-235;
ee) a heavy chain variable region comprising 1, 2, or 3 CDR sequences selected from SEQ ID NOs: 241-243;
ff) a heavy chain variable region comprising 1, 2, or 3 CDR sequences selected from SEQ ID NOs: 249-251;
gg) a heavy chain variable region comprising 1, 2, or 3 CDR sequences selected from SEQ ID NOs: 257-259;
hh) a heavy chain variable region comprising 1, 2, or 3 CDR sequences selected from SEQ ID NOs: 265-267;
ii) a heavy chain variable region comprising 1, 2, or 3 CDR sequences selected from SEQ ID NOs: 273-275;
jj) a heavy chain variable region comprising 1, 2, or 3 CDR sequences selected from SEQ ID NOs: 281-283;
kk) a heavy chain variable region comprising 1, 2, or 3 CDR sequences selected from SEQ ID NOs: 289-291;
ll) a heavy chain variable region comprising 1, 2, or 3 CDR sequences selected from SEQ ID NOs: 297-299;
mm) a heavy chain variable region comprising 1, 2, or 3 CDR sequences selected from SEQ ID NOs: 305-307;
nn) a heavy chain variable region comprising 1, 2, or 3 CDR sequences selected from SEQ ID NOs: 313-315;
oo) a heavy chain variable region comprising 1, 2, or 3 CDR sequences selected from SEQ ID NOs: 321-323;
pp) a heavy chain variable region comprising 1, 2, or 3 CDR sequences selected from SEQ ID NOs: 329-331;
qq) a heavy chain variable region comprising 1, 2, or 3 CDR sequences selected from SEQ ID NOs: 337-339; and
rr) a heavy chain variable region comprising 1, 2, or 3 CDR sequences selected from SEQ ID NOs: 9, 374 and 375.

In some embodiments, the antibody or antigen-binding fragment thereof provided herein comprises a light chain variable region selected from the group consisting of:
a) a light chain variable region comprising 1, 2, or 3 CDR sequences selected from SEQ ID NOs: 4-6;
b) a light chain variable region comprising 1, 2, or 3 CDR sequences selected from SEQ ID NOs: 12-14;
c) a light chain variable region comprising 1, 2, or 3 CDR sequences selected from SEQ ID NOs: 20-22;
d) a light chain variable region comprising 1, 2, or 3 CDR sequences selected from SEQ ID NOs: 28-30;
e) a light chain variable region comprising 1, 2, or 3 CDR sequences selected from SEQ ID NOs: 36-38;
f) a light chain variable region comprising 1, 2, or 3 CDR sequences selected from SEQ ID NOs: 44-46;
g) a light chain variable region comprising 1, 2, or 3 CDR sequences selected from SEQ ID NOs: 52-54;
h) a light chain variable region comprising 1, 2, or 3 CDR sequences selected from SEQ ID NOs: 60-62;
i) a light chain variable region comprising 1, 2, or 3 CDR sequences selected from SEQ ID NOs: 68-70;
j) a light chain variable region comprising 1, 2, or 3 CDR sequences selected from SEQ ID NOs: 76-78;
k) a light chain variable region comprising 1, 2, or 3 CDR sequences selected from SEQ ID NOs: 84-86;
l) a light chain variable region comprising 1, 2, or 3 CDR sequences selected from SEQ ID NOs: 92-94;
m) a light chain variable region comprising 1, 2, or 3 CDR sequences selected from SEQ ID NOs: 100-102, 108-110;
n) a light chain variable region comprising 1, 2, or 3 CDR sequences selected from SEQ ID NOs: 108-110;
o) a light chain variable region comprising 1, 2, or 3 CDR sequences selected from SEQ ID NOs: 116-118;
p) a light chain variable region comprising 1, 2, or 3 CDR sequences selected from SEQ ID NOs: 124-126;
q) a light chain variable region comprising 1, 2, or 3 CDR sequences selected from SEQ ID NOs: 132-134;
r) a light chain variable region comprising 1, 2, or 3 CDR sequences selected from SEQ ID NOs: 140-142;
s) a light chain variable region comprising 1, 2, or 3 CDR sequences selected from SEQ ID NOs: 148-150;
t) a light chain variable region comprising 1, 2, or 3 CDR sequences selected from SEQ ID NOs: 156-158;
u) a light chain variable region comprising 1, 2, or 3 CDR sequences selected from SEQ ID NOs: 164-166;
v) a light chain variable region comprising 1, 2, or 3 CDR sequences selected from SEQ ID NOs: 172-174;
w) a light chain variable region comprising 1, 2, or 3 CDR sequences selected from SEQ ID NOs: 180-181;
x) a light chain variable region comprising 1, 2, or 3 CDR sequences selected from SEQ ID NOs: 188-190;
y) a light chain variable region comprising 1, 2, or 3 CDR sequences selected from SEQ ID NOs: 196-198;
z) a light chain variable region comprising 1, 2, or 3 CDR sequences selected from SEQ ID NOs: 204-206;
aa) a light chain variable region comprising 1, 2, or 3 CDR sequences selected from SEQ ID NOs: 212-214;
bb) a light chain variable region comprising 1, 2, or 3 CDR sequences selected from SEQ ID NOs: 220-222;
cc) a light chain variable region comprising 1, 2, or 3 CDR sequences selected from SEQ ID NOs: 228-230;
dd) a light chain variable region comprising 1, 2, or 3 CDR sequences selected from SEQ ID NOs: 236-238;
ee) a light chain variable region comprising 1, 2, or 3 CDR sequences selected from SEQ ID NOs: 244-246;
ff) a light chain variable region comprising 1, 2, or 3 CDR sequences selected from SEQ ID NOs: 252-254;
gg) a light chain variable region comprising 1, 2, or 3 CDR sequences selected from SEQ ID NOs: 260-262;
hh) a light chain variable region comprising 1, 2, or 3 CDR sequences selected from SEQ ID NOs: 268-270;
ii) a light chain variable region comprising 1, 2, or 3 CDR sequences selected from SEQ ID NOs: 276-278;
jj) a light chain variable region comprising 1, 2, or 3 CDR sequences selected from SEQ ID NOs: 284-286;
kk) a light chain variable region comprising 1, 2, or 3 CDR sequences selected from SEQ ID NOs: 292-294;
ll) a light chain variable region comprising 1, 2, or 3 CDR sequences selected from SEQ ID NOs: 300-302;
mm) a light chain variable region comprising 1, 2, or 3 CDR sequences selected from SEQ ID NOs: 308-310;
nn) a light chain variable region comprising 1, 2, or 3 CDR sequences selected from SEQ ID NOs: 316-318;
oo) a light chain variable region comprising 1, 2, or 3 CDR sequences selected from SEQ ID NOs: 324-326;
pp) a light chain variable region comprising 1, 2, or 3 CDR sequences selected from SEQ ID NOs: 332-334;
qq) a light chain variable region comprising 1, 2, or 3 CDR sequences selected from SEQ ID NOs: 340-342;
rr) a light chain variable region comprising 1, 2, or 3 CDR sequences selected from SEQ ID NOs: 376, 13 and 14; and
ss) a light chain variable region comprising 1, 2, or 3 CDR sequences selected from SEQ ID NOs: 377, 45 and 46.

In some embodiments, the antibody or antigen-binding fragment thereof provided herein comprises:
a) a heavy chain variable region comprising 1, 2, or 3 CDR sequences selected from SEQ ID NO: 1, SEQ ID NO: 2, and SEQ ID NO: 3; and a light chain variable region comprising 1, 2, or 3 CDR sequences selected from SEQ ID NO: 4, SEQ ID NO: 5, and SEQ ID NO: 6;
b) a heavy chain variable region comprising 1, 2, or 3 CDR sequences selected from SEQ ID NO: 9, SEQ ID NO: 10, and SEQ ID NO: 11; and a light chain variable region comprising 1, 2, or 3 CDR sequences selected from SEQ ID NO: 12, SEQ ID NO: 13, and SEQ ID NO: 14;
c) a heavy chain variable region comprising 1, 2, or 3 CDR sequences selected from SEQ ID NO: 17, SEQ ID NO: 18, and SEQ ID NO: 19; and a light chain variable region comprising 1, 2, or 3 CDR sequences selected from SEQ ID NO: 20, SEQ ID NO: 21 and SEQ ID NO: 22;
d) a heavy chain variable region comprising 1, 2, or 3 CDR sequences selected from SEQ ID NO: 25, SEQ ID NO: 26, and SEQ ID NO: 27; and a light chain variable region comprising 1, 2, or 3 CDR sequences selected from SEQ ID NO: 28, SEQ ID NO: 29, and SEQ ID NO: 30;
e) a heavy chain variable region comprising 1, 2, or 3 CDR sequences selected from SEQ ID NO: 33, SEQ ID NO: 34, and SEQ ID NO: 35; and a light chain variable region comprising 1, 2, or 3 CDR sequences selected from SEQ ID NO: 36, SEQ ID NO: 37, and SEQ ID NO: 38;
f) a heavy chain variable region comprising 1, 2, or 3 CDR sequences selected from SEQ ID NO:41, SEQ ID NO: 42, and SEQ ID NO: 43; and a light chain variable region comprising 1, 2, or 3 CDR sequences selected from SEQ ID NO: 44, SEQ ID NO: 45, and SEQ ID NO:46;
g) a heavy chain variable region comprising 1, 2, or 3 CDR sequences selected from SEQ ID NO:49, SEQ ID NO: 50, and SEQ ID NO: 51; and a light chain variable region comprising 1, 2, or 3 CDR sequences selected from SEQ ID NO: 52, SEQ ID NO: 53, and SEQ ID NO: 54;
h) a heavy chain variable region comprising 1, 2, or 3 CDR sequences selected from SEQ ID NO: 57, SEQ ID NO: 58, and SEQ ID NO: 59; and a light chain variable region comprising 1, 2, or 3 CDR sequences selected from SEQ ID SEQ ID NO: 60, SEQ ID NO: 61, and SEQ ID NO: 62;
i) a heavy chain variable region comprising 1, 2, or 3 CDR sequences selected from SEQ ID NO: 65, SEQ ID NO: 66, and SEQ ID NO: 67; and a kappa light chain variable region comprising 1, 2, or 3 CDR sequences selected from SEQ ID NO: 68, SEQ ID NO: 69, and SEQ ID NO: 70;
j) a heavy chain variable region comprising 1, 2, or 3 CDR sequences selected from SEQ ID NO: 73, SEQ ID NO: 74, and SEQ ID NO: 75; and a kappa light chain variable region comprising 1, 2, or 3 CDR sequences selected from SEQ ID NO: 76, SEQ ID NO: 77, and SEQ ID NO: 78;
k) a heavy chain variable region comprising 1, 2, or 3 CDR sequences selected from SEQ ID NO: 81, SEQ ID NO: 82, and SEQ ID NO: 83; and a kappa light chain variable region comprising 1, 2, or 3 CDR sequences selected from SEQ ID NO: 84, SEQ ID NO: 85, and SEQ ID NO: 86;
l) a heavy chain variable region comprising 1, 2, or 3 CDR sequences selected from SEQ ID NO: 89, SEQ ID NO: 90, and SEQ ID NO: 91; and a light chain variable region comprising 1, 2, or 3 CDR sequences selected from SEQ ID NO: 92, SEQ ID NO: 93, and SEQ ID NO: 94;
m) a heavy chain variable region comprising 1, 2, or 3 CDR sequences selected from SEQ ID NO: 97, SEQ ID NO: 98, and SEQ ID NO: 99; and a light chain variable region comprising 1, 2, or 3 CDR sequences selected from SEQ ID NO: 100, SEQ ID NO: 101, and SEQ ID NO: 102;
n) a heavy chain variable region comprising 1, 2, or 3 CDR sequences selected from SEQ ID NO: 105, SEQ ID NO: 106, and SEQ ID NO: 107; and a light chain variable region comprising 1, 2, or 3 CDR sequences selected from SEQ ID NO: 108, SEQ ID NO: 109 and SEQ ID NO: 110;
o) a heavy chain variable region comprising 1, 2, or 3 CDR sequences selected from SEQ ID NO: 113, SEQ ID NO: 114, and SEQ ID NO: 115; and a light chain variable region comprising 1, 2, or 3 CDR sequences selected from SEQ ID NO: 116, SEQ ID NO: 117, and SEQ ID NO: 118;
p) a heavy chain variable region comprising 1, 2, or 3 CDR sequences selected from SEQ ID NO: 121, SEQ ID NO: 122, and SEQ ID NO: 123; and a light chain variable region comprising 1, 2, or 3 CDR sequences selected from SEQ ID NO: 124, SEQ ID NO: 125, and SEQ ID NO: 126;
q) a heavy chain variable region comprising 1, 2, or 3 CDR sequences selected from SEQ ID NO:129, SEQ ID NO: 130, and SEQ ID NO: 131; and a light chain variable region comprising 1, 2, or 3 CDR sequences selected from SEQ ID NO: 132, SEQ ID NO: 133, and SEQ ID NO:134;
r) a heavy chain variable region comprising 1, 2, or 3 CDR sequences selected from SEQ ID NO:137, SEQ ID NO: 138, and SEQ ID NO: 139; and a light chain variable region comprising 1, 2, or 3 CDR sequences selected from SEQ ID NO: 140, SEQ ID NO: 141, and SEQ ID NO: 142;
s) a heavy chain variable region comprising 1, 2, or 3 CDR sequences selected from SEQ ID NO: 145, SEQ ID NO: 146, and SEQ ID NO: 147; and a light chain variable region comprising 1, 2, or 3 CDR sequences selected from SEQ ID SEQ ID NO: 148, SEQ ID NO: 149, and SEQ ID NO: 150;
t) a heavy chain variable region comprising 1, 2, or 3 CDR sequences selected from SEQ ID NO: 153, SEQ ID NO: 154, and SEQ ID NO: 155; and a kappa light chain variable region comprising 1, 2, or 3 CDR sequences selected from SEQ ID NO: 156, SEQ ID NO: 157, and SEQ ID NO: 158;
u) a heavy chain variable region comprising 1, 2, or 3 CDR sequences selected from SEQ ID NO: 161, SEQ ID NO: 162, and SEQ ID NO: 163; and a kappa light chain variable region comprising 1, 2, or 3 CDR sequences selected from SEQ ID NO: 164, SEQ ID NO: 165, and SEQ ID NO: 166;
v) a heavy chain variable region comprising 1, 2, or 3 CDR sequences selected from SEQ ID NO: 169, SEQ ID NO: 170, and SEQ ID NO: 171; and a kappa light chain variable region comprising 1, 2, or 3 CDR sequences selected from SEQ ID NO: 172, SEQ ID NO: 173, and SEQ ID NO: 174;
w) a heavy chain variable region comprising 1, 2, or 3 CDR sequences selected from SEQ ID NO: 177, SEQ ID NO: 178, and SEQ ID NO: 179; and a light chain variable region comprising 1, 2, or 3 CDR sequences selected from SEQ ID NO: 180, SEQ ID NO: 181, and SEQ ID NO: 182;
x) a heavy chain variable region comprising 1, 2, or 3 CDR sequences selected from SEQ ID NO: 185, SEQ ID NO: 186, and SEQ ID NO: 187; and a light chain variable region comprising 1, 2, or 3 CDR sequences selected from SEQ ID NO: 188, SEQ ID NO: 189, and SEQ ID NO: 190;
y) a heavy chain variable region comprising 1, 2, or 3 CDR sequences selected from SEQ ID NO: 193, SEQ ID NO: 194, and SEQ ID NO: 195; and a light chain variable region comprising 1, 2, or 3 CDR sequences selected from SEQ ID NO: 196, SEQ ID NO: 197 and SEQ ID NO: 198;
z) a heavy chain variable region comprising 1, 2, or 3 CDR sequences selected from SEQ ID NO: 201, SEQ ID NO: 202, and SEQ ID NO: 203; and a light chain variable region comprising 1, 2, or 3 CDR sequences selected from SEQ ID NO: 204, SEQ ID NO: 205, and SEQ ID NO: 206;
aa) a heavy chain variable region comprising 1, 2, or 3 CDR sequences selected from SEQ ID NO: 209, SEQ ID NO: 210, and SEQ ID NO: 211; and a light chain variable region comprising 1, 2, or 3 CDR sequences selected from SEQ ID NO: 212, SEQ ID NO: 213, and SEQ ID NO: 214;
bb) a heavy chain variable region comprising 1, 2, or 3 CDR sequences selected from SEQ ID NO:217, SEQ ID NO: 218, and SEQ ID NO: 219; and a light chain variable region comprising 1, 2, or 3 CDR sequences selected from SEQ ID NO: 220, SEQ ID NO: 221, and SEQ ID NO:222;
cc) a heavy chain variable region comprising 1, 2, or 3 CDR sequences selected from SEQ ID NO:225, SEQ ID NO: 226, and SEQ ID NO: 227; and a light chain variable region comprising 1, 2, or 3 CDR sequences selected from SEQ ID NO: 228, SEQ ID NO: 229, and SEQ ID NO: 230;
dd) a heavy chain variable region comprising 1, 2, or 3 CDR sequences selected from SEQ ID NO: 233, SEQ ID NO: 234, and SEQ ID NO: 235; and a light chain variable region comprising 1, 2, or 3 CDR sequences selected from SEQ ID SEQ ID NO: 236, SEQ ID NO: 237, and SEQ ID NO: 238;
ee) a heavy chain variable region comprising 1, 2, or 3 CDR sequences selected from SEQ ID NO: 241, SEQ ID NO: 242, and SEQ ID NO: 243; and a kappa light chain variable region comprising 1, 2, or 3 CDR sequences selected from SEQ ID NO: 244, SEQ ID NO: 245, and SEQ ID NO: 246;
ff) a heavy chain variable region comprising 1, 2, or 3 CDR sequences selected from SEQ ID NO: 249, SEQ ID NO: 250, and SEQ ID NO: 251; and a kappa light chain variable region comprising 1, 2, or 3 CDR sequences selected from SEQ ID NO: 252, SEQ ID NO: 253, and SEQ ID NO: 254;
gg) a heavy chain variable region comprising 1, 2, or 3 CDR sequences selected from SEQ ID NO: 257, SEQ ID NO: 258, and SEQ ID NO: 259; and a kappa light chain variable region comprising 1, 2, or 3 CDR sequences selected from SEQ ID NO: 260, SEQ ID NO: 261, and SEQ ID NO: 262;
hh) a heavy chain variable region comprising 1, 2, or 3 CDR sequences selected from SEQ ID NO: 265, SEQ ID NO: 266, and SEQ ID NO: 267; and a light chain variable region comprising 1, 2, or 3 CDR sequences selected from SEQ ID NO: 268, SEQ ID NO: 269, and SEQ ID NO: 270;
ii) a heavy chain variable region comprising 1, 2, or 3 CDR sequences selected from SEQ ID NO: 273, SEQ ID NO: 274, and SEQ ID NO: 275; and a light chain variable region comprising 1, 2, or 3 CDR sequences selected from SEQ ID NO: 276, SEQ ID NO: 277, and SEQ ID NO: 278;
jj) a heavy chain variable region comprising 1, 2, or 3 CDR sequences selected from SEQ ID NO: 281, SEQ ID NO: 282, and SEQ ID NO: 283; and a light chain variable region comprising 1, 2, or 3 CDR sequences selected from SEQ ID NO: 284, SEQ ID NO: 285 and SEQ ID NO: 286;
kk) a heavy chain variable region comprising 1, 2, or 3 CDR sequences selected from SEQ ID NO: 289, SEQ ID NO: 290, and SEQ ID NO: 291; and a light chain variable region comprising 1, 2, or 3 CDR sequences selected from SEQ ID NO: 292, SEQ ID NO: 293, and SEQ ID NO: 294;
ll) a heavy chain variable region comprising 1, 2, or 3 CDR sequences selected from SEQ ID NO: 297, SEQ ID NO: 298, and SEQ ID NO: 299; and a light chain variable region comprising 1, 2, or 3 CDR sequences selected from SEQ ID NO: 300, SEQ ID NO: 301, and SEQ ID NO: 302;
mm) a heavy chain variable region comprising 1, 2, or 3 CDR sequences selected from SEQ ID NO:305, SEQ ID NO: 306, and SEQ ID NO: 307; and a light chain variable region comprising 1, 2, or 3 CDR sequences selected from SEQ ID NO: 308, SEQ ID NO: 309, and SEQ ID NO:310;
nn) a heavy chain variable region comprising 1, 2, or 3 CDR sequences selected from SEQ ID NO:313, SEQ ID NO: 314, and SEQ ID NO: 315; and a light chain variable region comprising 1, 2, or 3 CDR sequences selected from SEQ ID NO: 316, SEQ ID NO: 317, and SEQ ID NO: 318;
oo) a heavy chain variable region comprising 1, 2, or 3 CDR sequences selected from SEQ ID NO: 321, SEQ ID NO: 322, and SEQ ID NO: 323; and a light chain variable region comprising 1, 2, or 3 CDR sequences selected from SEQ ID SEQ ID NO: 324, SEQ ID NO: 325, and SEQ ID NO: 326;
pp) a heavy chain variable region comprising 1, 2, or 3 CDR sequences selected from SEQ ID NO: 329, SEQ ID NO: 330, and SEQ ID NO: 331; and a kappa light chain variable region comprising 1, 2, or 3 CDR sequences selected from SEQ ID NO: 332, SEQ ID NO: 333, and SEQ ID NO: 334;
qq) a heavy chain variable region comprising 1, 2, or 3 CDR sequences selected from SEQ ID NO: 337, SEQ ID NO: 338, and SEQ ID NO: 339; and a kappa light chain variable region comprising 1, 2, or 3 CDR sequences selected from SEQ ID NO: 340, SEQ ID NO: 341, and SEQ ID NO: 342; or
rr) a heavy chain variable region comprising 1, 2, or 3 CDR sequences selected from SEQ ID NO: 9, SEQ ID NO: 374, and SEQ ID NO: 375; and a kappa light chain variable region comprising 1, 2, or 3 CDR sequences selected from SEQ ID NO: 376, SEQ ID NO: 13, and SEQ ID NO: 14; or
ss) a heavy chain variable region comprising 1, 2, or 3 CDR sequences selected from SEQ ID NO: 41, SEQ ID NO: 42, and SEQ ID NO: 43; and a kappa light chain variable region comprising 1, 2, or 3 CDR sequences selected from SEQ ID NO: 377, SEQ ID NO: 45, and SEQ ID NO: 46.

In some embodiments, the antibody or antigen-binding fragment thereof provided herein comprises a heavy chain variable region selected from the group consisting of: SEQ ID NO: 7, SEQ ID NO: 15, SEQ ID NO: 23, SEQ ID NO: 31, SEQ ID NO: 39, SEQ ID NO: 47, SEQ ID NO: 55, SEQ ID NO: 63, SEQ ID NO: 71, SEQ ID NO: 79, SEQ ID NO: 87, SEQ ID NO: 95, SEQ ID NO: 103, SEQ ID NO: 111, SEQ ID NO: 119, SEQ ID NO: 127, SEQ ID NO: 135, SEQ ID NO: 143, SEQ ID NO: 151, SEQ ID NO: 159, SEQ ID NO: 167, SEQ ID NO: 175, SEQ ID NO: 183, SEQ ID NO: 191, SEQ ID NO: 199, SEQ ID NO: 207, SEQ ID NO: 215, SEQ ID NO: 223, SEQ ID NO: 231, SEQ ID NO: 239, SEQ ID NO: 247, SEQ ID NO: 255, SEQ ID NO: 263, SEQ ID NO: 271, SEQ ID NO: 279, SEQ ID NO: 287, SEQ ID NO: 295, SEQ ID NO: 303, SEQ ID NO: 311, SEQ ID NO: 319, SEQ ID NO: 327, SEQ ID NO: 335, SEQ ID NO: 343, SEQ ID NO: 347, and SEQ ID NO: 349 and the homologue sequences of at least 80% (e.g., at least 85%, 88%, 90%, 91%, 92%, 93%, 94%, 95%, 96%, 97%, 98%, 99%) sequence identity thereof.

In some embodiments, the antibody or antigen-binding fragment thereof provided herein comprises a light chain variable region selected from the group consisting of: SEQ ID NO: 8, SEQ ID NO: 16, SEQ ID NO: 24, SEQ ID NO: 32, SEQ ID NO: 40, SEQ ID NO: 48, SEQ ID NO: 56, SEQ ID NO: 64, SEQ ID NO: 72, SEQ ID NO: 80, SEQ ID NO: 88, SEQ ID NO: 96, SEQ ID NO: 104, SEQ ID NO: 112, SEQ ID NO: 120, SEQ ID NO: 128, SEQ ID NO: 136, SEQ ID NO: 144, SEQ ID NO: 152, SEQ ID NO: 160, SEQ ID NO: 168, SEQ ID NO: 1756, SEQ ID NO: 184, SEQ ID NO: 192, SEQ ID NO: 200, SEQ ID NO: 208, SEQ ID NO: 216, SEQ ID NO: 224, SEQ ID NO: 232, SEQ ID NO: 240, SEQ ID NO: 248, SEQ ID NO: 256, SEQ ID NO: 264, SEQ ID NO: 272, SEQ ID NO: 280, SEQ ID NO: 288, SEQ ID NO: 296, SEQ ID NO: 304, SEQ ID NO: 312, SEQ ID NO: 320, SEQ ID NO: 328, SEQ ID NO: 336, SEQ ID NO: 344, SEQ ID NO: 348, and SEQ ID NO: 350 and the homologue sequences of at least 80% (e.g., at least 85%, 88%, 90%, 91%, 92%, 93%, 94%, 95%, 96%, 97%, 98%, 99%) sequence identity thereof.

In some embodiments, the antibody or antigen-binding fragment thereof provided herein comprises:
a) a heavy chain variable region comprising SEQ ID NO: 7 and a light chain variable region comprising SEQ ID NO: 8;
b) a heavy chain variable region comprising SEQ ID NO: 15 and a light chain variable region comprising SEQ ID NO: 16;
c) a heavy chain variable region comprising SEQ ID NO: 23 and a light chain variable region comprising SEQ ID NO: 24;
d) a heavy chain variable region comprising SEQ ID NO: 31 and a light chain variable region comprising SEQ ID NO: 32;
e) a heavy chain variable region comprising SEQ ID NO: 39 and a light chain variable region comprising SEQ ID NO: 40;
f) a heavy chain variable region comprising SEQ ID NO: 47 and a light chain variable region comprising SEQ ID NO: 48;
g) a heavy chain variable region comprising SEQ ID NO: 55 and a light chain variable region comprising SEQ ID NO: 56;
h) a heavy chain variable region comprising SEQ ID NO: 63 and a light chain variable region comprising SEQ ID NO: 64;
i) a heavy chain variable region comprising SEQ ID NO: 71 and a light chain variable region comprising SEQ ID NO: 72;
j) a heavy chain variable region comprising SEQ ID NO: 79 and a light chain variable region comprising SEQ ID NO: 80;
k) a heavy chain variable region comprising SEQ ID NO: 87 and a light chain variable region comprising SEQ ID NO: 88;
l) a heavy chain variable region comprising SEQ ID NO: 95 and a light chain variable region comprising SEQ ID NO: 96;
m) a heavy chain variable region comprising SEQ ID NO: 103 and a light chain variable region comprising SEQ ID NO: 104;
n) a heavy chain variable region comprising SEQ ID NO: 111 and a light chain variable region comprising SEQ ID NO: 112;
o) a heavy chain variable region comprising SEQ ID NO: 119 and a light chain variable region comprising SEQ ID NO: 120;
p) a heavy chain variable region comprising SEQ ID NO: 127 and a light chain variable region comprising SEQ ID NO: 128;
q) a heavy chain variable region comprising SEQ ID NO: 135 and a light chain variable region comprising SEQ ID NO: 136;
r) a heavy chain variable region comprising SEQ ID NO: 143 and a light chain variable region comprising SEQ ID NO: 144;
s) a heavy chain variable region comprising SEQ ID NO: 151 and a light chain variable region comprising SEQ ID NO: 152;
t) a heavy chain variable region comprising SEQ ID NO: 159 and a light chain variable region comprising SEQ ID NO: 160;
u) a heavy chain variable region comprising SEQ ID NO: 167 and a light chain variable region comprising SEQ ID NO: 168;
v) a heavy chain variable region comprising SEQ ID NO: 175 and a light chain variable region comprising SEQ ID NO: 176;
w) a heavy chain variable region comprising SEQ ID NO: 183 and a light chain variable region comprising SEQ ID NO: 184;
x) a heavy chain variable region comprising SEQ ID NO: 191 and a light chain variable region comprising SEQ ID NO: 192;
y) a heavy chain variable region comprising SEQ ID NO: 199 and a light chain variable region comprising SEQ ID NO: 200;
z) a heavy chain variable region comprising SEQ ID NO: 207 and a light chain variable region comprising SEQ ID NO: 208;
aa) a heavy chain variable region comprising SEQ ID NO: 215 and a light chain variable region comprising SEQ ID NO: 216;
bb) a heavy chain variable region comprising SEQ ID NO: 223 and a light chain variable region comprising SEQ ID NO: 224;
cc) a heavy chain variable region comprising SEQ ID NO: 231 and a light chain variable region comprising SEQ ID NO: 232;
dd) a heavy chain variable region comprising SEQ ID NO: 239 and a light chain variable region comprising SEQ ID NO: 240;
ee) a heavy chain variable region comprising SEQ ID NO: 247 and a light chain variable region comprising SEQ ID NO: 248;
ff) a heavy chain variable region comprising SEQ ID NO: 255 and a light chain variable region comprising SEQ ID NO: 256;
gg) a heavy chain variable region comprising SEQ ID NO: 263 and a light chain variable region comprising SEQ ID NO: 264;
hh) a heavy chain variable region comprising SEQ ID NO: 271 and a light chain variable region comprising SEQ ID NO: 272;
ii) a heavy chain variable region comprising SEQ ID NO: 279 and a light chain variable region comprising SEQ ID NO: 280;
jj) a heavy chain variable region comprising SEQ ID NO: 287 and a light chain variable region comprising SEQ ID NO: 288;
kk) a heavy chain variable region comprising SEQ ID NO: 295 and a light chain variable region comprising SEQ ID NO: 296;
ll) a heavy chain variable region comprising SEQ ID NO: 303 and a light chain variable region comprising SEQ ID NO: 304;
mm) a heavy chain variable region comprising SEQ ID NO: 311 and a light chain variable region comprising SEQ ID NO: 312;
nn) a heavy chain variable region comprising SEQ ID NO: 319 and a light chain variable region comprising SEQ ID NO: 320;
oo) a heavy chain variable region comprising SEQ ID NO: 327 and a light chain variable region comprising SEQ ID NO: 328;
pp) a heavy chain variable region comprising SEQ ID NO: 335 and a light chain variable region comprising SEQ ID NO:336;
qq) a heavy chain variable region comprising SEQ ID NO: 343 and a light chain variable region comprising SEQ ID NO: 344;
rr) a heavy chain variable region comprising SEQ ID NO: 347 and a light chain variable region comprising SEQ ID NO: 348; or
ss) a heavy chain variable region comprising SEQ ID NO: 349 and a light chain variable region comprising SEQ ID NO: 350.

In some embodiments, the antibody or antigen-binding fragment thereof is humanized.

In some embodiments, the antibody or antigen-binding fragment thereof comprises a heavy chain variable region comprising SEQ ID NO: 378 (EVOLVESGGGLXQPGXSLRLSCXTSGFTLSDYYMSWVRQXPGKGLEWVXF MRNKANXYTTEYSASVRGRFTISRDTSKSXIYLQMNSLXXEDTAVYYCVRDR XGRPFAYWGQGTLVTVSS), wherein the X at position i (i=12, 16, 23, 40, 49, 57, 80, 89, 90 and 103) of SEQ ID NO: 378 is referred as X_{Hi}, wherein X_{H12} is V or I, X_{H16} is G or R, X_{H23} is A or T, X_{H40} is A or P, X_{H49} is G or S, X_{H57} is A or G, X_{H80} is I or T, X_{H89} is R or K, X_{H90} is A or T, X_{H103} is D or E; and a light chain variable region comprising SEQ ID NO: 379 (DIXMTQSPXSLXXXXGXXXXIXCKSSQSLLNXINQKNFLTWYXQKPGXXPX LLIYWASTRESGVPXRFSGSGSGTDFTLXISXXXXEDLXXYYCQNDYTYPLTF GQGTKLEIK), wherein the X at position i (i=3, 9, 12, 13, 14, 15, 17, 18, 19, 20, 22, 32, 43, 48, 49, 51, 66, 80, 83, 84, 85, 86, 90 and 91) of SEQ ID NO: 379 is referred as X_{Li}, wherein X_{L3} is V or Q, X_{L9} is D, L or S, X_{L12} is A, S or P, X_{L13} is A or V, X_{L14} is S or T, X_{L15} is L, V or P, X_{L17} is D or E, X_{L18} is R or P, X_{L19} is A or V, X_{L20} is S or T, X_{L22} is N, T or S, X_{L32} is A or S, X_{L43} is Q or L, X_{L48} is Q or K, X_{L49} is A, P or S, X_{L51} is K or Q, X_{L66} is S or D, X_{L80} is K or T, X_{L83} is R or S, X_{L84} is L or V, X_{L85} is Q or E, X_{L86} is A or P, X_{L90} is A or G, X_{L91} is T or V.

In some embodiments, the antibody or antigen-binding fragment thereof comprises a heavy chain variable region comprising SEQ ID NO: 380 (QVQLQESGPGLVKPSXTLSLTCXVXGYSITSDYAWNWIRQXPGKGLEWIGYI SHSGSTSYNPSLKSRVTISRDTSKNQFSLKLSSVTAADTAVYYCARSLGRRWY FDVWGQGTTVTVSS), wherein the X at position i (i=16, 23, 25 and 41) of SEQ ID NO: 380 is referred as X_{Hi}, wherein X_{H16} is E or Q, X_{H23} is A or T, X_{H25} is S or Y, X_{H41} is H or P; and a light chain variable region comprising SEQ ID NO: 381 (DIXMTQSPXSLXXXXGXXXXIXCKSSQSLLXSSTQKNYLAWYXQKPGXXPX LLIYFASTRDSGVPXRFSGSGSGTDFTLXISXXXXEDLXXYFCQQHYIIPFTFG QGTKLEIK), wherein the X at position i (i=3, 9, 12, 13, 14, 15, 17, 18, 19, 20, 22, 31, 43, 48, 49, 51, 66, 80, 83, 84, 85, 86, 90 and 91) of SEQ ID NO: 381 is referred as X_{Li}, wherein X_{L3} is V or Q, X_{L9} is D, L or S, X_{L12} is A, S or P, X_{L13} is A or V, X_{L14} IS S or T, X_{L15} is L, V or P, X_{L17} is D or E, X_{L18} is R or P, X_{L19} is A or V, X_{L20} is S or T, X_{L22} is N, T or S, X_{L31} is N or Q, X_{L43} is Q or L, X_{L48} is Q or K, X_{L49} is A, P or S, X_{L51} is K or Q, X_{L66} is S or D, X_{L80} is K or T, X_{L83} is R or S, X_{L84} is L or V, X_{L85} is Q or E, X_{L86} is A or P, X_{L90} is A or G, X_{L91} is T or V.

In some embodiments, the antibody or antigen-binding fragment thereof comprises:
(i) a heavy chain variable region comprising 1, 2, or 3 CDR sequences shown as SEQ ID NO: 9, SEQ ID NO: 10 or 374 (MRNKANAYTT), and SEQ ID NO: 11 or 375 (VRDREGRPFAY), respectively; and a kappa light chain variable region comprising 1, 2, or 3 CDR sequences shown as SEQ ID NO: 12 or 376 (QSLLNAINQKNF), SEQ ID NO: 13, and SEQ ID NO: 14, respectively; or
(ii) a heavy chain variable region comprising 1, 2, or 3 CDR sequences shown as SEQ ID NO: 41, SEQ ID NO: 42, and SEQ ID NO: 43, respectively; and a kappa light chain variable region comprising 1, 2, or 3 CDR sequences shown as SEQ ID NO: 44 or 377 (QSLLQSSTQKNY), SEQ ID NO: 45, and SEQ ID NO: 46, respectively.

In some embodiments, the antibody or antigen-binding fragment thereof comprises a heavy chain variable region comprising a sequence selected from SEQ ID NO: 351, 353, 355, 357, 358, 360, 362, 364, 365, 367 and 370; and a light chain variable region comprising a sequence selected from SEQ ID NO: 352, 354, 356, 359, 361, 363, 366, 368, 369, 371, 372 and 373.

In some embodiments, the antibody or antigen-binding fragment thereof provided herein comprises:
a) a heavy chain variable region comprising SEQ ID NO: 351 and a light chain variable region comprising SEQ ID NO: 352;
b) a heavy chain variable region comprising SEQ ID NO: 353 and a light chain variable region comprising SEQ ID NO: 354;
c) a heavy chain variable region comprising SEQ ID NO: 355 and a light chain variable region comprising SEQ ID NO: 352;
d) a heavy chain variable region comprising SEQ ID NO: 355 and a light chain variable region comprising SEQ ID NO: 356;
e) a heavy chain variable region comprising SEQ ID NO: 357 and a light chain variable region comprising SEQ ID NO: 352;
f) a heavy chain variable region comprising SEQ ID NO: 357 and a light chain variable region comprising SEQ ID NO: 354;
g) a heavy chain variable region comprising SEQ ID NO: 358 and a light chain variable region comprising SEQ ID NO: 359;
h) a heavy chain variable region comprising SEQ ID NO: 360 and a light chain variable region comprising SEQ ID NO: 361;
i) a heavy chain variable region comprising SEQ ID NO: 362 and a light chain variable region comprising SEQ ID NO: 359;
j) a heavy chain variable region comprising SEQ ID NO: 362 and a light chain variable region comprising SEQ ID NO: 363;
k) a heavy chain variable region comprising SEQ ID NO: 364 and a light chain variable region comprising SEQ ID NO: 359;
l) a heavy chain variable region comprising SEQ ID NO: 364 and a light chain variable region comprising SEQ ID NO: 361;
m) a heavy chain variable region comprising SEQ ID NO: 365 and a light chain variable region comprising SEQ ID NO: 366;
n) a heavy chain variable region comprising SEQ ID NO: 367 and a light chain variable region comprising SEQ ID NO: 368;
o) a heavy chain variable region comprising SEQ ID NO: 367 and a light chain variable region comprising SEQ ID NO: 369;
p) a heavy chain variable region comprising SEQ ID NO: 367 and a light chain variable region comprising SEQ ID NO: 366;
q) a heavy chain variable region comprising SEQ ID NO: 370 and a light chain variable region comprising SEQ ID NO: 369;
r) a heavy chain variable region comprising SEQ ID NO: 370 and a light chain variable region comprising SEQ ID NO: 366;
s) a heavy chain variable region comprising SEQ ID NO: 365 and a light chain variable region comprising SEQ ID NO: 371;
t) a heavy chain variable region comprising SEQ ID NO: 367 and a light chain variable region comprising SEQ ID NO: 372;
u) a heavy chain variable region comprising SEQ ID NO: 367 and a light chain variable region comprising SEQ ID NO: 373;
v) a heavy chain variable region comprising SEQ ID NO: 367 and a light chain variable region comprising SEQ ID NO: 371;
w) a heavy chain variable region comprising SEQ ID NO: 370 and a light chain variable region comprising SEQ ID NO: 373; or
x) a heavy chain variable region comprising SEQ ID NO: 370 and a light chain variable region comprising SEQ ID NO: 371.

In some embodiments, the antibody or antigen-binding fragment thereof provided herein further comprises one or more amino acid residue substitutions or modifications yet retains specific binding affinity to human CD276.

In some embodiments, the substitution is in one or more CDR sequences, and/or in one or more of the VH or VL sequences but not in any of the CDR sequences.

In some embodiments, the antibody or antigen-binding fragment thereof provided herein further comprises an immunoglobulin constant region, optionally a constant region of human Ig, or optionally a constant region of human IgG.

In some embodiments, the constant region comprises a constant region of human IgG1, IgG2, IgG3, or IgG4.

In some embodiments, the antibody or antigen-binding fragment thereof provided herein is a chimeric or humanized.

In some embodiments, the antibody or antigen-binding fragment thereof provided herein is a Fab, a Fab′, a F(ab′)₂, a Fd, an Fv fragment, a disulfide stabilized Fv fragment (dsFv), a (dsFv)₂, a single-chain antibody molecule (scFv), a camelized single domain antibody, a nanobody, a domain antibody, and a bivalent domain antibody.

In some embodiments, the antibody or antigen-binding fragment is linked to one or more conjugates. In some embodiments, the conjugate is covalently attached either directly or via a linker. In some embodiments, the conjugate comprises a clearance-modifying agent, a chemotherapeutic agent, a toxin, a radioactive isotope, a lanthanide, a luminescent label, a fluorescent label, an enzyme-substrate label, a DNA-alkylators, a topoisomerase inhibitor, a tubulin-binders, or other anticancer drugs.

In some embodiments, the antibody or antigen-binding fragment is capable of specifically binding to CD276. In some embodiments, the CD276 are derived from human. In some embodiments, the CD276 is a recombinant CD276 or a CD276 expressed on a cell surface.

In one aspect, the present disclosure provides an antibody or antigen-binding fragment thereof, which competes for the same epitope with the antibody or antigen-binding fragment thereof provided herein.

In one aspect, the present disclosure provides a pharmaceutical composition comprising the antibody or antigen-binding fragment thereof provided herein, and a pharmaceutically acceptable carrier.

In some embodiments, the pharmaceutical composition provided herein further comprises a second therapeutic agent.

In some embodiments, the second therapeutic agent is an antagonist against one or more immunoinhibitory molecules.

In some embodiments, the second therapeutic agent is a PD-L1 antagonist.

In some embodiments, the PD-L1 antagonist is a PD-L1 antibody or antigen-binding fragment thereof.

In one aspect, the present disclosure provides an isolated polynucleotide encoding the antibody or antigen-binding fragment thereof provided herein.

In one aspect, the present disclosure provides a vector comprising the isolated polynucleotide provided herein.

In one aspect, the present disclosure provides a host cell comprising the vector provided herein.

In one aspect, the present disclosure provides a method of expressing the antibody or antigen-binding fragment thereof provided herein, comprising culturing the host cell provided herein under the condition at which the vector provided herein is expressed.

In one aspect, the present disclosure provides a method of treating a disease or condition in a subject that would benefit from modulation of CD276 activity, comprising administering to the subject a therapeutically effective amount of the antibody or antigen-binding fragment thereof provided herein or the pharmaceutical composition provided herein.

In some embodiments, the disease or condition is a CD276 related disease or condition.

In some embodiments, the disease or condition is cancer, adaptive immune disease, autoimmune disease, inflammatory disease, or infectious disease.

In some embodiments, the cancer is adrenal gland tumor, an AIDS-associated cancer, an alveolar soft part sarcoma, an astrocytic tumor, bladder cancer, bone cancer, a brain and spinal cord cancer, a metastatic brain tumor, a breast cancer, a carotid body tumors, a cervical cancer, a chondrosarcoma, a chordoma, a chromophobe renal cell carcinoma, a clear cell carcinoma, a colon cancer, a colorectal cancer, a cutaneous benign fibrous histiocytoma, a desmoplastic small round cell tumor, an ependymoma, a Ewing's tumor, an extraskeletal myxoid chondrosarcoma, a fibrogenesis imperfecta ossium, a fibrous dysplasia of the bone, a gallbladder or bile duct cancer, gastric cancer, a gestational trophoblastic disease, a germ cell tumor, a head and neck cancer, hepatocellular carcinoma, an islet cell tumor, a Kaposi's Sarcoma, a kidney cancer, a leukemia, a lipoma/benign lipomatous tumor, a liposarcoma/malignant lipomatous tumor, a liver cancer, a lymphoma, a lung cancer, a medulloblastoma, a melanoma, a meningioma, a multiple endocrine neoplasia, a multiple myeloma, a myelodysplastic syndrome, a neuroblastoma, a neuroendocrine tumors, an ovarian cancer, a pancreatic cancer, a papillary thyroid carcinoma, a parathyroid tumor, a pediatric cancer, a peripheral nerve sheath tumor, a phaeochromocytoma, a pituitary tumor, a prostate cancer, a posterior uveal melanoma, a rare hematologic disorder, a renal metastatic cancer, a rhabdoid tumor, a rhabdomysarcoma, a sarcoma, a skin cancer, a soft-tissue sarcoma, a squamous cell cancer, a stomach cancer, a synovial sarcoma, a testicular cancer, a thymic carcinoma, a thymoma, a thyroid metastatic cancer, and a uterine cancer, optionally, wherein the cancer is chemoresistant.

In some embodiments, the disease or condition is hematological cancer selected from B-cell lymphomas, such as Hodgkin's lymphoma, non-Hodgkin's lymphoma (NHL), acute lymphocytic leukemia (ALL), acute myeloid leukemia (AML), chronic lymphocytic leukemia (CLL), chronic myelogenous leukemia (CML), multiple myeloma (MM), diffuse large B cell lymphoma (DLBCL), Marginal zone B-cell lymphoma (MZL), Mantle cell lymphoma (MCL), Richter's syndrome, Burkitt's lymphoma or follicular lymphoma.

In some embodiments, the subject is human.

In some embodiments, the method provided herein comprises administering to the subject a therapeutically effective amount of one or more therapeutic agent. In some embodiments, said therapeutic agent is a chemotherapeutic agent, a radiation therapeutic agent, a hormonal therapeutic agent, a toxin or an immunotherapeutic agent.

In some embodiments, the second therapeutic agent is a PD-L1 antagonist.

In some embodiments, the PD-L1 antagonist is an anti-PD-L1 antibody or antigen-binding fragment thereof.

In some embodiments, the administration is via oral, nasal, intravenous, subcutaneous, sublingual, or intramuscular administration.

In some embodiments, said method further comprises administration of one or more additional cancer therapies selected from the group consisting of chemotherapy, immunotherapy, radiation therapy, hormonal therapy, and surgery.

In one aspect, the present disclosure provides a method of modulating CD276 activity in a CD276-expressing cell, comprising exposing the CD276-expressing cell to the antibody or antigen-binding fragment thereof provided herein.

In one aspect, the present disclosure provides a method of detecting presence or amount of CD276 in a sample, comprising contacting the sample with the antibody or antigen-binding fragment thereof provided herein, and determining the presence or the amount of CD276 in the sample.

In one aspect, the present disclosure provides a method of diagnosing a CD276 related disease or condition in a subject, comprising: a) obtaining a sample from the subject; b) contacting the sample obtained from the subject with the antibody or antigen-binding fragment thereof provided herein; c) determining presence or amount of CD276 in the sample; and d) correlating the presence or the amount of CD276 to existence or status of the CD276 related disease or condition in the subject.

In one aspect, the present disclosure provides use of the antibody or antigen-binding fragment thereof provided herein in the manufacture of a medicament for treating a CD276 related disease or condition in a subject.

In some embodiments, the medicament further comprises a second therapeutic agent. In some embodiments, the second therapeutic agent is a chemotherapeutic agent, a radiation therapeutic agent, a hormonal therapeutic agent, a toxin or an immunotherapeutic agent.

In some embodiments, the second therapeutic agent is a PD-L1 antagonist. In some embodiments, the PD-L1 antagonist is an anti-PD-L1 antibody or antigen-binding fragment thereof.

In one aspect, the present disclosure provides use of the antibody or antigen-binding fragment thereof provided herein in the manufacture of a diagnostic reagent for diagnosing a CD276 related disease or condition.

In one aspect, the present disclosure provides a kit comprising the antibody or antigen-binding fragment thereof provided herein, useful in detecting CD276, optionally recombinant CD276, CD276 expressed on cell surface, or CD276-expressing cells.

In one aspect, the present disclosure provides a chimeric antigen receptor (CAR) comprising an antigen binding domain, a transmembrane domain, a costimulatory signaling region, and a TCR signaling domain, wherein the antigen binding domain specifically binds to CD276 and comprises an antigen binding fragment provided herein. In some embodiments, the antigen binding fragment is a Fab or a scFv.

In some embodiments, the CAR provided herein is bispecific.

In some embodiments, the CAR is capable of further specifically binding to a second antigen other than CD276, or a second epitope on CD276. In some embodiments, the second antigen is a tumor antigen.

In some embodiments, the TCR signaling domain is selected from the group consisting of: an intracellular signal regions sequence of CD3ζ, FccRIγ, CD27, CD28, CD137, CD134, MyD88, CD40, CD278, TLRs, or a combination thereof.

In some embodiments, the transmembrane region comprises a transmembrane region of CD3, CD4, CD8 or CD28.

In one aspect, the present disclosure provides a nucleic acid sequence encoding the chimeric antigen receptor (CAR) provided herein. In one aspect, the present disclosure provides a cell comprising the nucleic acid sequence provided herein. In one aspect, the present disclosure provides a cell genetically modified to express the CAR provided herein. In some embodiments, the cell is immune cell, optionally, wherein the immune cell is T lymphocyte, NK cell, monocyte, macrophage or NKT lymphocyte.

In one aspect, the present disclosure provides a vector comprising the nucleic acid sequence provided herein.

In one aspect, the present disclosure provides a method for stimulating a T cell-mediated immune response to a CD276-expressing cell or tissue in a mammal, the method comprising administering to the mammal an effective amount of a cell genetically modified to express the CAR provided herein.

In one aspect, the present disclosure provides a method of treating a mammal having a CD276 related disease or condition, comprising administering to the mammal an effective amount of a cell provided herein, thereby treating the mammal. In some embodiments, the cell is an autologous T cell.

In some embodiments, the CD276 related disease or condition is cancer.

In some embodiments, the mammal is a human subject.

In one aspect, the present disclosure provides use of a cell genetically modified to express the CAR provided herein in the manufacture of a medicament for stimulating a T cell-mediated immune response to a CD276-expressing cell or tissue in a mammal.

In one aspect, the present disclosure provides use a cell provided herein in the manufacture of a medicament for treating a mammal having a CD276 related disease or condition.

### BRIEF DESCRIPTION OF THE DRAWINGS

The drawings are for illustration purposes only not for limitation.

FIG. 1 shows the binding affinity of the anti-CD276 antibodies provided herein on SKOV3 cells as measured by FACs analysis.

FIG. 2A-2E show binding of 6-D8-E7-A11 to several cancer cell lines that express B7H3 as measured by FACS analysis.

FIG. 3A-3C show ADCC effect of the anti-CD276 antibodies provided herein on SKOV3 cells.

FIGS. 4A and 4B show CDC effect of the anti-CD276 antibodies provided herein on CHO-S-hCD276 cells.

FIG. 5A-5E show indirect ADC cytotoxicity effect of the anti-CD276 antibodies provided herein on SKOV3 cells.

FIG. 6 shows in vivo efficacy of the anti-CD276 antibodies provided herein in inhibiting the tumor growth in the mouse model inoculated with MC-38-hCD276 (B7H3) tumor cells.

FIG. 7 shows effects on tumor growth in subcutaneous calu-6 model in balb/c nude mice (mean±sem).

FIG. 8 shows IL2 release by T cell activation in MLR assay.

FIG. 9 shows IFNγ release by T cell activation in MLR assay.

FIG. 10 shows the binding affinity on SKOV3 of humanized antibodies derived from 30-C7-C11-D4.

FIG. 11 shows binding affinity on SKOV3 of humanized antibodies derived from 10-G6-C4-B2.

FIG. 12 shows effects on tumor growth in subcutaneous Calu-6 model in Balb/c nude mice (Mean±SEM).

### DETAILED DESCRIPTION OF THE INVENTION

The following description of the disclosure is merely intended to illustrate various embodiments of the disclosure. As such, the specific modifications discussed are not to be construed as limitations on the scope of the disclosure. It will be apparent to one skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety.

### Definitions

The term “antibody” as used herein includes any immunoglobulin, monoclonal antibody, polyclonal antibody, multivalent antibody, bivalent antibody, or monovalent antibody that binds to a specific antigen. A native intact antibody comprises two heavy (H) chains and two light (L) chains. Mammalian heavy chains are classified as alpha, delta, epsilon, gamma, and mu, each heavy chain consists of a variable region (V_{H}) and a first, second, and third constant region (C_{H1}, C_{H2}, C_{H3}, respectively); mammalian light chains are classified as λ or κ, while each light chain consists of a variable region (V_{L}) and a constant region. The antibody has a “Y” shape, with the stem of the Y consisting of the second and third constant regions of two heavy chains bound together via disulfide bonding. Each arm of the Y includes the variable region and first constant region of a single heavy chain bound to the variable and constant regions of a single light chain. The variable regions of the light and heavy chains are responsible for antigen binding. The variable regions in both chains generally contain three highly variable loops called the complementarity determining regions (CDRs) (light chain CDRs including LCDR1, LCDR2, and LCDR3, heavy chain CDRs including HCDR1, HCDR2, and HCDR3). CDR boundaries for the antibodies and antigen-binding domains disclosed herein may be defined or identified by the conventions of Kabat, IMGT, AbM, Chothia, or A1-Lazikani (A1-Lazikani, B., Chothia, C., Lesk, A. M., J. Mol. Biol., 273 (4), 927 (1997); Chothia, C. et al., J Mol Biol. Dec 5; 186 (3): 651-63 (1985); Chothia, C. and Lesk, A. M., J. Mol. Biol., 196,901 (1987); N. R. Whitelegg et al, Protein Engineering, v13 (12), 819-824 (2000); Chothia, C. et al., Nature. Dec 21-28; 342 (6252): 877-83 (1989); Kabat E. A. et al., National Institutes of Health, Bethesda, Md. (1991); Marie-Paule Lefranc et al, Developmental and Comparative Immunology, 27:55-77 (2003); Marie-Paule Lefranc et al, Immunome Research, 1 (3), (2005); Marie-Paule Lefranc, Molecular Biology of B cells (second edition), chapter 26, 481-514, (2015)). The three CDRs are interposed between flanking stretches known as framework regions (FRs), which are more highly conserved than the CDRs and form a scaffold to support the hypervariable loops. The constant regions of the heavy and light chains are not involved in antigen-binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of alpha, delta, epsilon, gamma, and mu heavy chains, respectively. Several of the major antibody classes are divided into subclasses such as IgG1 (gamma1 heavy chain), IgG2 (gamma2 heavy chain), IgG3 (gamma3 heavy chain), IgG4 (gamma4 heavy chain), IgA1 (alpha1 heavy chain), or IgA2 (alpha2 heavy chain).

The term “antibody molecule” as used herein refers to an antigen-binding protein or polypeptide comprising at least one antibody fragment (such as CDR, and/or variable region sequence). An antibody molecule includes, for example, a monoclonal antibody, an antibody fragment or domain, a fusion protein comprising an antibody fragment or domain, a polypeptide complex comprising an antibody fragment or domain, and so on.

The term “antigen-binding domain” (e.g. CD276-binding domain) as used herein refers to an antibody fragment formed from a portion of an antibody comprising one or more CDRs, or any other antibody fragment that binds to an antigen but does not comprise an intact native antibody structure. Examples of antigen-binding domain include, without limitation, a Fab, a Fab′, a F(ab′)₂, an Fv fragment, a disulfide stabilized Fv fragment (dsFv), a (dsFv)₂, a single-chain antibody molecule (scFv), a camelized single domain antibody, a nanobody, a domain antibody, and a bivalent domain antibody. An antigen-binding domain is capable of binding to the same antigen to which the parent antibody binds. In certain embodiments, an antigen-binding domain may comprise one or more CDRs from a particular human antibody grafted to a framework region from one or more different human antibodies. For more and detailed formats of antigen-binding domain are described in Spiess et al, 2015, and Brinkman et al., mAbs, 9 (2), pp. 182-212 (2017), which are incorporated herein by entirety reference.

“Fab” with regard to an antibody refers to that portion of the antibody consisting of a single light chain (both variable and constant regions) bound to the variable region and first constant region of a single heavy chain by a disulfide bond.

“Fab′” refers to a Fab fragment that includes a portion of the hinge region.

“F(ab′)₂” refers to a dimer of Fab′.

A “fragment difficult (Fd)” with regard to an antibody refers to the amino-terminal half of the heavy chain fragment that can be combined with the light chain to form Fab. For example, Fd fragment may consists of the V_{H} and CH1 domains

“Fv” with regard to an antibody refers to the smallest fragment of the antibody to bear the complete antigen-binding site. An Fv fragment consists of the variable region of a single light chain bound to the variable region of a single heavy chain. A number of Fv designs have been provided, including dsFvs, in which the association between the two domains is enhanced by an introduced disulphide bond; and scFvs can be formed using a peptide linker to bind the two domains together as a single polypeptide. Fvs constructs containing a variable domain of a heavy or light immunoglobulin chain associated to the variable and constant domain of the corresponding immunoglobulin heavy or light chain have also been produced.

“Single-chain Fv antibody” or “scFv” refers to an engineered antibody consisting of a light chain variable region and a heavy chain variable region connected to one another directly or via a peptide linker sequence (Huston J S et al. Proc Natl Acad Sci USA, 85: 5879 (1988)).

A “dsFv” refers to a disulfide-stabilized Fv fragment that the linkage between the variable region of a single light chain and the variable region of a single heavy chain is a disulfide bond. In some embodiments, a “(dsFv)₂” or “(dsFv-dsFv′)” comprises three peptide chains: two V_{H} moieties linked by a peptide linker (e.g., a long flexible linker) and bound to two V_{L} moieties, respectively, via disulfide bridges. In some embodiments, dsFv-dsFv′ is bispecific in which each disulfide paired heavy and light chain has a different antigen specificity.

“Fc” with regard to an antibody refers to that portion of the antibody consisting of the second and third constant regions of a first heavy chain bound to the second and third constant regions of a second heavy chain via disulfide bonding. The Fc portion of the antibody is responsible for various effector functions such as antibody-dependent cell-mediated cytotoxicity (ADCC), and complement dependent cytotoxicity (CDC), but does not function in antigen binding.

“Camelized single domain antibody,” “heavy chain antibody,” or “HCAb” refers to an antibody that contains two V_{H} domains and no light chains (Riechmann L. and Muyldermans S., J Immunol Methods. Dec 10; 231 (1-2): 25-38 (1999); Muyldermans S., J Biotechnol. Jun; 74 (4): 277-302 (2001); WO94/04678; WO94/25591; U.S. Pat. No. 6,005,079). Heavy chain antibodies were originally derived from Camelidae (camels, dromedaries, and llamas). Although devoid of light chains, camelized antibodies have an authentic antigen-binding repertoire (Hamers-Casterman C. et al., Nature. Jun 3; 363 (6428): 446-8 (1993); Nguyen V K. et al. “Heavy-chain antibodies in Camelidae; a case of evolutionary innovation,” Immunogenetics. Apr; 54 (1): 39-47 (2002); Nguyen V K. et al. Immunology. May; 109 (1): 93-101 (2003)). The variable domain of a heavy chain antibody (VHH domain) represents the smallest known antigen-binding unit generated by adaptive immune responses (Koch-Nolte F. et al., FASEB J. Nov; 21 (13): 3490-8. Epub 2007 Jun. 15 (2007)).

A “nanobody” refers to an antibody fragment that consists of a VHH domain from a heavy chain antibody and two constant domains, CH2 and CH3.

A “domain antibody” refers to an antibody fragment containing only the variable region of a heavy chain or the variable region of a light chain. In certain instances, two or more V_{H} domains are covalently joined with a peptide linker to create a bivalent or multivalent domain antibody. The two V_{H} domains of a bivalent domain antibody may target the same or different antigens.

The term “chimeric” as used herein, means an antibody or antigen-binding domain, having a portion of heavy and/or light chain derived from one species, and the rest of the heavy and/or light chain derived from a different species. In an illustrative example, a chimeric antibody may comprise a constant region derived from human and a variable region from a non-human animal, such as from mouse. In some embodiments, the non-human animal is a mammal, for example, a mouse, a rat, a rabbit, a goat, a sheep, a guinea pig, or a hamster.

The term “humanized” as used herein means that the antibody or antigen-binding domain comprises CDRs derived from non-human animals, FR regions derived from human, and when applicable, the constant regions derived from human.

The term “operably link” or “operably linked” refers to a juxtaposition, with or without a spacer or a linker or an intervening sequence, of two or more biological sequences of interest in such a way that they are in a relationship permitting them to function in an intended manner. When used with respect to polypeptides, it is intended to mean that the polypeptide sequences are linked in such a way that permits the linked product to have the intended biological function. For example, an antibody variable region may be operably linked to a constant region so as to provide for a stable product with antigen-binding activity. For another example, an antigen-binding domain can be operably linked to another antigen-binding domain with an intervening sequence there between, and such intervening sequence can be a spacer or can comprise a much longer sequence such as a constant region of an antibody. The term may also be used with respect to polynucleotides. For one instance, when a polynucleotide encoding a polypeptide is operably linked to a regulatory sequence (e.g., promoter, enhancer, silencer sequence, etc.), it is intended to mean that the polynucleotide sequences are linked in such a way that permits regulated expression of the polypeptide from the polynucleotide.

The term “fusion” or “fused” when used with respect to amino acid sequences (e.g. peptide, polypeptide or protein) refers to combination of two or more amino acid sequences, for example by chemical bonding or recombinant means, into a single amino acid sequence which does not exist naturally. A fusion amino acid sequence may be produced by genetic recombination of two encoding polynucleotide sequences, and can be expressed by a method of introducing a construct containing the recombinant polynucleotides into a host cell.

An “antigen” as used herein refers to a compound, composition, peptide, polypeptide, protein or substance that can stimulate the production of antibodies or a T cell response in cell culture or in an animal, including compositions (such as one that includes a cancer-specific protein) that are added to a cell culture (such as a hybridoma), or injected or absorbed into an animal. An antigen reacts with the products of specific humoral or cellular immunity (such as an antibody), including those induced by heterologous antigens.

The term “CD276 protein” or “B7-H3 protein” as used herein is intended to encompass any form of CD276, for example, 1) native unprocessed CD276 molecule, “full-length” CD276 chain or naturally occurring variants of CD276, including, for example, splice variants or allelic variants; 2) any form of CD276 that results from processing in the cell; or 3) full length, a fragment (e.g., a truncated form, an extracellular/transmembrane domain) or a modified form (e.g. a mutated form, a glycosylated/PEGylated, a His-tag/immunofluorescence fused form) of CD276 subunit generated through recombinant method.

The term “anti-CD276 antibody”, “anti-CD276 binding domain” or “CD276-binding domain” refers to an antibody or antigen-binding domain that is capable of specifically binding to CD276 (e.g. human, monkey or mouse CD276).

The term “specific binding” or “specifically binds” as used herein refers to a non-random binding reaction between two molecules, such as for example between an antibody and an antigen.

Binding of the antibodies to recombinant CD276 or CD276 expressed on surface of cells can also be represented by “half maximal effective concentration” (EC₅₀) value, which refers to the concentration of an antibody where 50% of its maximal effect (e.g., binding or inhibition etc.) is observed. The EC₅₀ value can be measured by methods known in the art, for example, sandwich assay such as ELISA, Western Blot, flow cytometry assay, and other binding assays. In certain embodiments, the antibodies and the fragments thereof provided herein specifically bind to recombinant human CD276 at an EC₅₀ (i.e. 50% binding concentration) of no more than 0.05 nM, no more than 0.06 nM, no more than 0.07 nM, no more than 0.08 nM, no more than 0.09 nM, no more than 0.1 nM, no more than 0.2 nM, no more than 0.3 nM, no more than 0.4 nM, no more than 0.5 nM, no more than 0.6 nM, no more than 0.7 nM, no more than 0.8 nM, no more than 0.9 nM, no more than 1 nM, no more than 1.5 nM, no more than 2 nM, no more than 2.5 nM, no more than 3.5 nM, no more than 3 nM, no more than 4 nM, no more than 4.5 nM, no more than 5 nM, no more than 6 nM, no more than 7 nM, no more than 8 nM, no more than 9 nM, or no more than 10 nM, by flow cytometry assay.

The ability to “block binding” or “compete for the same epitope” as used herein refers to the ability of an antibody or antigen-binding domain to inhibit the binding interaction between two molecules (e.g. human CD276 and its binding ligand, e.g. TLT-2) to any detectable degree. In certain embodiments, an antibody or antigen-binding domain that blocks binding between two molecules inhibits the binding interaction between the two molecules by at least 85%, or at least 90%. In certain embodiments, this inhibition may be greater than 85%, or greater than 90%.

The term “epitope” as used herein refers to the specific group of atoms or amino acids on an antigen to which an antibody binds. Epitopes can be formed both from contiguous amino acids (also called linear or sequential epitope) or noncontiguous amino acids juxtaposed by tertiary folding of a protein (also called configurational or conformational epitope). Epitopes formed from contiguous amino acids are typically arranged linearly along the primary amino acid residues on the protein and the small segments of the contiguous amino acids can be digested from an antigen binding with major histocompatibility complex (MHC) molecules or retained on exposure to denaturing solvents whereas epitopes formed by tertiary folding are typically lost on treatment with denaturing solvents. An epitope typically includes at least 3, and more usually, at least 5, about 7, or about 8-10 amino acids in a unique spatial conformation. Two antibodies may bind the same or a closely related epitope within an antigen if they exhibit competitive binding for the antigen. For example, if an antibody or antigen-binding domain blocks binding of a reference antibody to the antigen by at least 85%, or at least 90%, or at least 95%, then the antibody or antigen-binding domain may be considered to bind the same/closely related epitope as the reference antibody.

A “conservative substitution” with reference to amino acid sequence refers to replacing an amino acid residue with a different amino acid residue having a side chain with similar physiochemical properties. For example, conservative substitutions can be made among amino acid residues with hydrophobic side chains (e.g. Met, Ala, Val, Leu, and Ile), among residues with neutral hydrophilic side chains (e.g. Cys, Ser, Thr, Asn and Gln), among residues with acidic side chains (e.g. Asp, Glu), among amino acids with basic side chains (e.g. His, Lys, and Arg), or among residues with aromatic side chains (e.g. Trp, Tyr, and Phe). As known in the art, conservative substitution usually does not cause significant change in the protein conformational structure, and therefore could retain the biological activity of a protein.

The term “homolog” and “homologous” as used herein are interchangeable and refer to nucleic acid sequences (or its complementary strand) or amino acid sequences that have sequence identity of at least 80% (e.g., at least 85%, 88%, 90%, 91%, 92%, 93%, 94%, 95%, 96%, 97%, 98%, 99%) to another sequences when optimally aligned.

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). Conservative substitution of the amino acid residues may or may not be considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al, J. Mol. Biol., 215:403-410 (1990); Stephen F. et al, Nucleic Acids Res., 25:3389-3402 (1997)), ClustalW2 (available on the website of European Bioinformatics Institute, see also, Higgins D. G. et al, Methods in Enzymology, 266:383-402 (1996); Larkin M. A. et al, Bioinformatics (Oxford, England), 23 (21): 2947-8 (2007)), and ALIGN or Megalign (DNASTAR) software. Those skilled in the art may use the default parameters provided by the tool, or may customize the parameters as appropriate for the alignment, such as for example, by selecting a suitable algorithm.

“Effector functions” as used herein refer to biological activities attributable to the binding of Fc region of an antibody to its effectors such as C1 complex, Fc receptor and effector cell (e.g., macrophage). Exemplary effector functions include: complement dependent cytotoxicity (CDC) induced by interaction of antibodies and C1q on the C1 complex; antibody-dependent cell-mediated cytotoxicity (ADCC) induced by binding of Fc region of an antibody to Fc receptor on an effector cell; and antibody-dependent cellular phagocytosis (ADCP) induced by binding of Fc region of an antibody to phagocytosis. It has become well established that the specific glycan structures associated with the conserved bi-antennary glycan in the Fc-CH2 domain can strongly influence the interaction with the FcyRs that mediate ADCC and ADCP and with C1q binding, the initial binding event leading to CDC (see Reusch D, Tejada M L. Fc glycans of therapeutic antibodies as critical quality attributes. Glycobiology 2015; 25:1325-34).

“Treating” or “treatment” of a condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof.

The term “subject” or “individual” or “animal” or “patient” as used herein refers to human or non-human animal, including a mammal or a primate, in need of diagnosis, prognosis, amelioration, prevention and/or treatment of a disease or disorder. Mammalian subjects include humans, monkeys, domestic animals, farm animals, and zoo, sports, or pet animals such as dogs, cats, guinea pigs, rabbits, rats, mice, horses, swine, cows, bears, and so on.

The term “vector” as used herein refers to a vehicle into which a polynucleotide encoding a protein may be operably inserted so as to bring about the expression of that protein. A vector may be used to transform, transduce, or transfect a host cell so as to bring about expression of the genetic element it carries within the host cell. Examples of vectors include plasmids, phagemids, cosmids, and artificial chromosomes such as yeast artificial chromosome (YAC), bacterial artificial chromosome (BAC), or P1-derived artificial chromosome (PAC), bacteriophages such as lambda phage or M13 phage, and animal viruses. Categories of animal viruses used as vectors include retrovirus (including lentivirus), adenovirus, adeno-associated virus, herpesvirus (e.g., herpes simplex virus), poxvirus, baculovirus, papillomavirus, and papovavirus (e.g., SV40). A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating. A vector can be an expression vector or a cloning vector.

The phrase “host cell” as used herein refers to a cell into which an exogenous polynucleotide and/or a vector has been introduced.

A “CD276-related” disease or condition as used herein refers to any disease or condition caused by, exacerbated by, or otherwise linked to increased or decreased expression or activities of CD276. In some embodiments, the CD276 related condition is immune-related disorder, such as, for example, cancer, autoimmune disease, inflammatory disease or infectious disease.

“Cancer” as used herein refers to any medical condition characterized by malignant cell growth or neoplasm, abnormal proliferation, infiltration or metastasis, and includes both solid tumors and non-solid cancers (hematologic malignancies) such as leukemia. As used herein “solid tumor” refers to a solid mass of neoplastic and/or malignant cells. Examples of cancer or tumors include adrenal gland tumor, an AIDS-associated cancer, an alveolar soft part sarcoma, an astrocytic tumor, bladder cancer, bone cancer, a brain and spinal cord cancer, a metastatic brain tumor, a breast cancer, a carotid body tumors, a cervical cancer, a chondrosarcoma, a chordoma, a chromophobe renal cell carcinoma, a clear cell carcinoma, a colon cancer, a colorectal cancer, a cutaneous benign fibrous histiocytoma, a desmoplastic small round cell tumor, an ependymoma, a Ewing's tumor, an extraskeletal myxoid chondrosarcoma, a fibrogenesis imperfecta ossium, a fibrous dysplasia of the bone, a gallbladder or bile duct cancer, gastric cancer, a gestational trophoblastic disease, a germ cell tumor, a head and neck cancer, hepatocellular carcinoma, an islet cell tumor, a Kaposi's Sarcoma, a kidney cancer, a leukemia, a lipoma/benign lipomatous tumor, a liposarcoma/malignant lipomatous tumor, a liver cancer, a lymphoma, a lung cancer, a medulloblastoma, a melanoma, a meningioma, a multiple endocrine neoplasia, a multiple myeloma, a myelodysplastic syndrome, a neuroblastoma, a neuroendocrine tumors, an ovarian cancer, a pancreatic cancer, a papillary thyroid carcinoma, a parathyroid tumor, a pediatric cancer, a peripheral nerve sheath tumor, a phaeochromocytoma, a pituitary tumor, a prostate cancer, a posterior uveal melanoma, a rare hematologic disorder, a renal metastatic cancer, a rhabdoid tumor, a rhabdomysarcoma, a sarcoma, a skin cancer, a soft-tissue sarcoma, a squamous cell cancer, a stomach cancer, a synovial sarcoma, a testicular cancer, a thymic carcinoma, a thymoma, a thyroid metastatic cancer, and a uterine cancer.

In certain embodiments, the hematological malignancies includes B-cell lymphomas, optionally Hodgkin's lymphoma, non-Hodgkin's lymphoma (NHL), acute lymphocytic leukemia (ALL), acute myeloid leukemia (AML), chronic lymphocytic leukemia (CLL), chronic myelogenous leukemia (CML), multiple myeloma (MM), diffuse large B cell lymphoma (DLBCL), Marginal zone B-cell lymphoma (MZL), Mantle cell lymphoma (MCL), Richter's syndrome, Burkitt's lymphoma or follicular lymphoma.

In certain embodiments, the cancer is selected from gastric cancer, breast cancer, head and neck cancer, pancreatic cancer, and colon cancer. In certain embodiments, the cancer is selected from a lymphoma, Hodgkin's lymphoma, non-Hodgkin's lymphoma and B-cell lymphoma.

In certain embodiments, the cancer is chemoresistant. The term “chemoresistant cancer” as used herein refers to a type of cancer that are not responsive to the effects of chemotherapy. For example, a cancer that has been responding to a chemotherapy or a combination of different chemotherapies suddenly begins to grow can be referred to as a chemoresistant cancer.

The term “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

### Anti-CD276 Antibody

The present disclosure provides anti-CD276 antibodies and antigen-binding fragments thereof comprising one or more (e.g. 1, 2, 3, 4, 5, or 6) CDR sequences of an anti-CD276 antibody 9-E8-F9-C10, 10-G6-C4-B2, 18-F9-D8-G7, 9-G2-H6-E4, 20-F8-B5-G2, 30-C7-C11-D4, 23-F10-G4-F11, 6-H11-G5-D8, 27-E7-D8-C7, 30-E2-G7-G7, 5-D1-G6-D9, 3-C2-C3-E7, 11-G10-B4-B11, 16-C6-F7-F5, 22-E11-C3-F2, 24-C10-F9-G7, 25-C8-D7-C5, 4-D5-B9-B11, 10-B9-D10-A12, 15-G1-D1-E3, 8-B4-F5-E11, 6-F3-G2-G1, 9-B9-H11-G7, 9-G12-D6-A11, 13-A8-C4-G1, 15-C8-B5-G7, 28-G2-E6-B10, 3-G7-D8-D3, 14-E7-G9-D4, 20-C5-D7-D3, 8-C3-E3-F3, 2-A7-B10-A3, 26-D2-D6-B12, 11-C12-F4-F6, 16-G3-D10-C10, 27-F8-E10-E11, 7-E1-F8-F6, 25-D3-G4-C6, 13-E4-G9-A4, 20-A2-D10-G8, 3-F2-E7-F9, 6-D8-E7-A11, and 21-B3-B1-H5. In certain embodiments, the anti-CD276 antibodies and antigen-binding fragments thereof provided herein is capable of specifically binding to CD276. Optionally, the CD276 are derived from human, monkey or mouse. In certain embodiments, the CD276 is a recombinant CD276 or a CD276 expressed on a cell surface.

All of the anti-CD276 antibodies and antigen-binding fragments thereof provided herein are mouse monoclonal antibodies. Table 1 shows the CDR sequences of these 43 anti-CD276 antibodies according to IMGT numbering system. The heavy chain and light chain variable region sequences are also provided below.

TABLE 1
CDR sequences of the anti-CD276 antibodies

| Antibody Clone | | CDR1 | CDR2 | CDR3 |
|---|---|---|---|---|
| 9-E8-F9-C10 | VH | SEQ ID NO: 1 | SEQ ID NO: 2 | SEQ ID NO: 3 |
| | | GYAFSDYW | ICPGSGNI | TRLITRVIDY |
| | VL | SEQ ID NO: 4 | SEQ ID NO: 5 | SEQ ID NO: 6 |
| | | SSVNY | DTS | FQGSGYPLT |
| 10-G6-C4-B2 | VH | SEQ ID NO: 9 | SEQ ID NO: 10 | SEQ ID NO: 11 |
| | | GFTLSDYY | MRNKANGYTT | VRDRDGRPFAY |
| | VL | SEQ ID NO: 12 | SEQ ID NO: 13 | SEQ ID NO: 14 |
| | | QSLLNSINQKNF | WAS | QNDYTYPLT |
| 18-F9-D8-G7 | VH | SEQ ID NO: 17 | SEQ ID NO: 18 | SEQ ID NO: 19 |
| | | GYTFTDYA | INTYSGDT | ARGGGNYPYWYFDV |
| | VL | SEQ ID NO: 20 | SEQ ID NO: 21 | SEQ ID NO: 22 |
| | | SSVSSSY | STS | QQFSVYPLT |
| 9-G2-H6-E4 | VH | SEQ ID NO: 25 | SEQ ID NO: 26 | SEQ ID NO: 27 |
| | | GYSFTGYF | INPNNGDT | GRGGLPFGALDY |
| | VL | SEQ ID NO: 28 | SEQ ID NO: 29 | SEQ ID NO: 30 |
| | | QSLLYSRNQKNY | WAS | QQYHSYPYT |
| 20-F8-B5-G2 | VH | SEQ ID NO: 33 | SEQ ID NO: 34 | SEQ ID NO: 35 |
| | | GDSITSAY | ISNSGST | VRGERYYGYGAY |
| | VL | SEQ ID NO: 36 | SEQ ID NO: 37 | SEQ ID NO: 38 |
| | | KSLLHSNGNTYL Y | RMS | MQHLEYPFT |
| 30-C7-C11-D4 | VH | SEQ ID NO: 41 | SEQ ID NO: 42 | SEQ ID NO: 43 |
| | | GYSITSDYA | SHSGST | ARSLGRRWYFDV |
| | VL | SEQ ID NO: 44 | SEQ ID NO: 45 | SEQ ID NO: 46 |
| | | QSLLNSSTQKNY | FAS | QQHYIIPFT |
| 23-F10-G4-F11 | VH | SEQ ID NO: 49 | SEQ ID NO: 50 | SEQ ID NO: 51 |
| | | GFNIKDYP | FDPDNGNV | SSPYGAMDY |
| | VL | SEQ ID NO: 52 | SEQ ID NO: 53 | SEQ ID NO: 54 |
| | | SSVSY | DTS | QQGSSNPRT |
| 6-H11-G5-D8 | VH | SEQ ID NO: 57 | SEQ ID NO: 58 | SEQ ID NO: 59 |
| | | GYTFTSYD | IFPGNGDT | ARDLRLGTFDY |
| | VL | SEQ ID NO: 60 | SEQ ID NO: 61 | SEQ ID NO: 62 |
| | | SSVSY | DTS | QQGSSNPRT |
| 27-E7-D8-C7 | VH | SEQ ID NO: 65 | SEQ ID NO: 66 | SEQ ID NO: 67 |
| | | GYTFTDYA | INTYSDNT | ARGLLLRSQLGVYFDY |
| | VL | SEQ ID NO: 68 | SEQ ID NO: 69 | SEQ ID NO: 70 |
| | | SSVSY | ATS | QQWSSNPWT |
| 30-E2-G7-G7 | VH | SEQ ID NO: 73 | SEQ ID NO: 74 | SEQ ID NO: 75 |
| | | GFTFSYYA | INGDGSHT | ASLLQLGPF |
| | VL | SEQ ID NO: 76 | SEQ ID NO: 77 | SEQ ID NO: 78 |
| | | QNVGTN | SAS | QQYNNYPLT |
| 5-D1-G6-D9 | VH | SEQ ID NO: 81 | SEQ ID NO: 82 | SEQ ID NO: 83 |
| | | GLTFSTNY | IYGGNGGT | TSHDYYGGPMDY |
| | VL | SEQ ID NO: 84 | SEQ ID NO: 85 | SEQ ID NO: 86 |
| | | QDISNY | FTS | QQTNTLPWT |
| 3-C2-C3-E7 | VH | SEQ ID NO: 89 | SEQ ID NO: 90 | SEQ ID NO: 91 |
| | | GYTFTENT | INPNTGGT | ARRGTNWEHWYFDV |
| | VL | SEQ ID NO: 92 | SEQ ID NO: 93 | SEQ ID NO: 94 |
| | | SSVSY | DTS | QQWSSNPLT |
| 11-G10-B4-B11 | VH | SEQ ID NO: 97 | SEQ ID NO: 98 | SEQ ID NO: 99 |
| | | GYSITSDYA | ISYSGST | ARGGPWFAY |
| | VL | SEQ ID NO: 100 | SEQ ID NO: 101 | SEQ ID NO: 102 |
| | | QSL VHSNGNTY | KVS | SQSTHVPFT |
| 16-C6-F7-F5 | VH | SEQ ID NO: 105 | SEQ ID NO: 106 | SEQ ID NO: 107 |
| | | GYTFTKYG | IWTYTGKP | ARDDGYYYTMNY |
| | VL | SEQ ID NO: 108 | SEQ ID NO: 109 | SEQ ID NO: 110 |
| | | QSLLHTNGDTY | KVS | SQSTHIPWT |
| 22-E11-C3-F2 | VH | SEQ ID NO: 113 | SEQ ID NO: 114 | SEQ ID NO: 115 |
| | | GYSFTNYG | NPYTGEP | GRDDGYHYTMNY |
| | VL | SEQ ID NO: 116 | SEQ ID NO: 117 | SEQ ID NO: 118 |
| | | QSLIHSNGDTY | KVS | SQSTYVPWT |
| 24-C10-F9-G7 | VH | SEQ ID NO: 121 | SEQ ID NO: 122 | SEQ ID NO: 123 |
| | | GYTFTDHT | ISTYYGNT | ARASYGSGYGPYYWY |
| | | | | FDV |
| | VL | SEQ ID NO: 124 | SEQ ID NO: 125 | SEQ ID NO: 126 |
| | | EKIYSY | NAN | QHHYGTPVT |
| 25-C8-D7-C5 | VH | SEQ ID NO: 129 | SEQ ID NO: 130 | SEQ ID NO: 131 |
| | | GFTFSTYA | ISGGGIDT | ARQGSKGWGAMDY |
| | VL | SEQ ID NO: 132 | SEQ ID NO: 133 | SEQ ID NO: 134 |
| | | SSVSF | ETS | QQWSSNPLT |
| 4-D5-B9-B11 | VH | SEQ ID NO: 137 | SEQ ID NO: 138 | SEQ ID NO: 139 |
| | | GFTFSSYA | ISGGGI | ARQGSKGWDAMDY |
| | VL | SEQ ID NO: 140 | SEQ ID NO: 141 | SEQ ID NO: 142 |
| | | SSVSF | ETS | QQWSSNPLT |
| 10-B9-D10-A12 | VH | SEQ ID NO: 145 | SEQ ID NO: 146 | SEQ ID NO: 147 |
| | | GYTFTDYT | ISTYYGNT | ARASYGSGYGPYYWY |
| | | | | FDV |
| | VL | SEQ ID NO: 148 | SEQ ID NO: 149 | SEQ ID NO: 150 |
| | | EKIYSY | NAN | QHHYGTPVT |
| 15-G1-D1-E3 | VH | SEQ ID NO: 153 | SEQ ID NO: 154 | SEQ ID NO: 155 |
| | | GYTFTSYW | IHPKSGTV | ARGENYDYRPQFVY |
| | VL | SEQ ID NO: 156 | SEQ ID NO: 157 | SEQ ID NO: 158 |
| | | SQHSTYT | LKKDGSH | GVGDTIKEQFVYV |
| 8-B4-F5-E11 | VH | SEQ ID NO: 161 | SEQ ID NO: 162 | SEQ ID NO: 163 |
| | | GDTFTSYW | IHPDSGTR | CSRSYGSRLAWFAY |
| | VL | SEQ ID NO: 164 | SEQ ID NO: 165 | SEQ ID NO: 166 |
| | | TGAVTTSNF | GTN | ALWYTNHFI |
| 6-F3-G2-G1 | VH | SEQ ID NO: 169 | SEQ ID NO: 170 | SEQ ID NO: 171 |
| | | DYTFTNYW | IHPNSGTT | ASHYGNSYSAMDY |
| | VL | SEQ ID NO: 172 | SEQ ID NO: 173 | SEQ ID NO: 174 |
| | | TGAVTTSNY | GTS | ALWYSTHYV |
| 9-B9-H11-G7 | VH | SEQ ID NO: 177 | SEQ ID NO: 178 | SEQ ID NO: 179 |
| | | GYSITSGYY | YISYDGTN | ARGDSNSYYFDY |
| | VL | SEQ ID NO: 180 | SEQ ID NO: 181 | SEQ ID NO: 182 |
| | | TGAVTTSNY | GTS | ALWYRTHYV |
| 9-G12-D6-A11 | VH | SEQ ID NO: 185 | SEQ ID NO: 186 | SEQ ID NO: 187 |
| | | GYSITSDYY | ISYDGTN | ARGKDGSYYFDY |
| | VL | SEQ ID NO: 188 | SEQ ID NO: 189 | SEQ ID NO: 190 |
| | | TGAVTTSNY | GTS | ALWYRTHYV |
| 13-A8-C4-G1 | VH | SEQ ID NO: 193 | SEQ ID NO: 194 | SEQ ID NO: 195 |
| | | SGYIFTSNW | IHPNSGTT | AGPTTTRRFYPMDY |
| | VL | SEQ ID NO: 196 | SEQ ID NO: 197 | SEQ ID NO: 198 |
| | | TGAVTTSNY | GTN | AL WYSNHFI |
| 15-C8-B5-G7 | VH | SEQ ID NO: 201 | SEQ ID NO: 202 | SEQ ID NO: 203 |
| | | DYTFTSHWMHW | IHPNSGTT | AWGSSNSYFYAMAY |
| | VL | SEQ ID NO: 204 | SEQ ID NO: 205 | SEQ ID NO: 206 |
| | | TGAVTTSNY | GTN | ALWFSNHL V |
| 28-G2-E6-B10 | VH | SEQ ID NO: 209 | SEQ ID NO: 210 | SEQ ID NO: 211 |
| | | GYTFTSYW | IHPNSDTT | ARGGLGPHYFVY |
| | VL | SEQ ID NO: 212 | SEQ ID NO: 213 | SEQ ID NO: 214 |
| | | TGAVTTSNY | GTS | ALWYSPHYV |
| 3-G7-D8-D3 | VH | SEQ ID NO: 217 | SEQ ID NO: 218 | SEQ ID NO: 219 |
| | | GYTFTTYW | IYPGDGDT | ARPYDGYYIDYPMDS |
| | VL | SEQ ID NO: 220 | SEQ ID NO: 221 | SEQ ID NO: 222 |
| | | SRINY | DTS | HQRSSYPFT |
| 14-E7-G9-D4 | VH | SEQ ID NO: 225 | SEQ ID NO: 226 | SEQ ID NO: 227 |
| | | GYTFTNYI | INPYNDGI | TRWGNYVFYYTMDY |
| | VL | SEQ ID NO: 228 | SEQ ID NO: 229 | SEQ ID NO: 230 |
| | | SSVSY | ATS | QQWSGNPPLT |
| 20-C5-D7-D3 | VH | SEQ ID NO: 233 | SEQ ID NO: 234 | SEQ ID NO: 235 |
| | | GFTFRSYG | ISGGGNYT | ARHEGLPLDY |
| | VL | SEQ ID NO: 236 | SEQ ID NO: 237 | SEQ ID NO: 238 |
| | | ENIYSY | NAK | QHHYGTPTWT |
| 8-C3-E3-F3 | VH | SEQ ID NO: 241 | SEQ ID NO: 242 | SEQ ID NO: 243 |
| | | GFTFSNYG | IGSYGGNTY | ARDIYYGNSEAWFAY |
| | VL | SEQ ID NO: 244 | SEQ ID NO: 245 | SEQ ID NO: 246 |
| | | KSLLHSNGITY | QMS | AQNLELPWT |
| 2-A7-B10-A3 | VH | SEQ ID NO: 249 | SEQ ID NO: 250 | SEQ ID NO: 251 |
| | | GYTFTDYA | INTNYVNT | ARGGKYGNYVFFFAY |
| | VL | SEQ ID NO: 252 | SEQ ID NO: 253 | SEQ ID NO: 254 |
| | | QDISNY | YTS | QQGNTLPYT |
| 26-D2-D6-B12 | VH | SEQ ID NO: 257 | SEQ ID NO: 258 | SEQ ID NO: 259 |
| | | GYIFRNYG | INSYTGKPTY | ARDDGYWYTMDY |
| | VL | SEQ ID NO: 260 | SEQ ID NO: 261 | SEQ ID NO: 262 |
| | | QSL VHSNGHTY | KVS | SQSTHVPWT |
| 11-C12-F4-F6 | VH | SEQ ID NO: 265 | SEQ ID NO: 266 | SEQ ID NO: 267 |
| | | GYTFRNFG | INPYTGEP | ARDDGYWYTMDY |
| | VL | SEQ ID NO: 268 | SEQ ID NO: 269 | SEQ ID NO: 270 |
| | | QSL VHSNGITY | KVS | SQSTHVPWT |
| 16-G3-D10-C10 | VH | SEQ ID NO: 273 | SEQ ID NO: 274 | SEQ ID NO: 275 |
| | | GYTFRNYG | INPYTGEP | ARDDGYWYTMDY |
| | VL | SEQ ID NO: 276 | SEQ ID NO: 277 | SEQ ID NO: 278 |
| | | QSL VHSNGITY | KVS | SQSTHVPWT |
| 27-F8-E10-E11 | VH | SEQ ID NO: 281 | SEQ ID NO: 282 | SEQ ID NO: 283 |
| | | GYTFTEYA | INPNIGGT | ARFL VPSTEGFTY |
| | VL | SEQ ID NO: 284 | SEQ ID NO: 285 | SEQ ID NO: 286 |
| | | SSVSY | DTS | QQWSSNPLT |
| 7-E1-F8-F6 | VH | SEQ ID NO: 289 | SEQ ID NO: 290 | SEQ ID NO: 291 |
| | | GYTFTDYV | IYPRSGNT | ARFDIA YSFDY |
| | VL | SEQ ID NO: 292 | SEQ ID NO: 293 | SEQ ID NO: 294 |
| | | QSL VHSNGVTY | KVS | SQGTHVPWT |
| 25-D3-G4-C6 | VH | SEQ ID NO: 297 | SEQ ID NO: 298 | SEQ ID NO: 299 |
| | | GYSFTDYI | INPYYNST | ARDSFDWYFGV |
| | VL | SEQ ID NO: 300 | SEQ ID NO: 301 | SEQ ID NO: 302 |
| | | QSL VHSNGNTY | KVS | SQSTHVPPLT |
| 13-E4-G9-A4 | VH | SEQ ID NO: 305 | SEQ ID NO: 306 | SEQ ID NO: 307 |
| | | GFTFITYA | ISGGGIDT | TRQGSQGWGAMDY |
| | VL | SEQ ID NO: 308 | SEQ ID NO: 309 | SEQ ID NO: 310 |
| | | SRVSF | ETS | QQWSSNPLT |
| 20-A2-D10-G8 | VH | SEQ ID NO: 313 | SEQ ID NO: 314 | SEQ ID NO: 315 |
| | | GYTFTDYT | ISTYYGNT | ARASYGSGYGPYYWY |
| | | | | FDV |
| | VL | SEQ ID NO: 316 | SEQ ID NO: 317 | SEQ ID NO: 318 |
| | | EKIYSY | NAN | QHHYGSPVT |
| 3-F2-E7-F9 | VH | SEQ ID NO: 321 | SEQ ID NO: 322 | SEQ ID NO: 323 |
| | | GYTFTDYT | ISTYYGNT | ARASYGSGYGPYYWY |
| | | | | FDV |
| | VL | SEQ ID NO: 324 | SEQ ID NO: 325 | SEQ ID NO: 326 |
| | | EKIYSY | NAN | QHHYGTPVT |
| 6-D8-E7-A11 | VH | SEQ ID NO: 329 | SEQ ID NO: 330 | SEQ ID NO: 331 |
| | | GYTFTNYW | IHPNSGTT | AGYYGSSFSWFTY |
| | VL | SEQ ID NO: 332 | SEQ ID NO: 333 | SEQ ID NO: 334 |
| | | TGAVTTSNF | GTS | ALWYSTHYV |
| 21-B3-B1-H5 | VH | SEQ ID NO: 337 | SEQ ID NO: 338 | SEQ ID NO: 339 |
| | | GYIFRNYG | INSYTGKP | ARDDGYWYTMDY |
| | VL | SEQ ID NO: 340 | SEQ ID NO: 341 | SEQ ID NO: 342 |
| | | QSL VHSNGHTY | KVS | SQSTHVPWT |

Heavy or light chain variable region sequences of 9-E8-F9-C10, 10-G6-C4-B2, 18-F9-D8-G7, 9-G2-H6-E4, 20-F8-B5-G2, 30-C7-C11-D4, 23-F10-G4-F11, 6-H11-G5-D8, 27-E7-D8-C7, 30-E2-G7-G7, 5-D1-G6-D9, 3-C2-C3-E7, 11-G10-B4-B11, 16-C6-F7-F5, 22-E11-C3-F2, 24-C10-F9-G7, 25-C8-D7-C5, 4-D5-B9-B11, 10-B9-D10-A12, 15-G1-D1-E3, 8-B4-F5-E11, 6-F3-G2-G1, 9-B9-H11-G7, 9-G12-D6-A11, 13-A8-C4-G1, 15-C8-B5-G7, 28-G2-E6-B10, 3-G7-D8-D3, 14-E7-G9-D4, 20-C5-D7-D3, 8-C3-E3-F3, 2-A7-B10-A3, 26-D2-D6-B12, 11-C12-F4-F6, 16-G3-D10-C10, 27-F8-E10-E11, 7-E1-F8-F6, 25-D3-G4-C6, 13-E4-G9-A4, 20-A2-D10-G8, 3-F2-E7-F9, 6-D8-E7-A11, and 21-B3-B1-H5 antibodies are provided in Table 2.

TABLE 2
Variable region sequences of the anti-CD276 antibodies

| Clone | VH | VL |
|---|---|---|
| 9-E8-F9-C10 | SEQ ID NO: 7 | SEQ ID NO: 8 |
| | EVQLQQSGAELVRPGTSVKISC | ENVLTQSPAIMSASPGEKVTM |
| | KASGYAFSDYWLSWVKQRPG | TCSASSSVNYMHWYQQKSSTS |
| | HGLEWIGDICPGSGNIYYNERF | PKLWIYDTSKLSSGVPGRFSGS |
| | KDKVILTADRSSSTAYLQFSSL | GSGNSYSLTISSMEAEDVATY |
| | TSEDSVVYFCTRLITRVIDYWG | YCFQGSGYPLTFGAGTKLELK |
| | QGTSVTVST | |
| 10-G6-C4-B2 | SEQ ID NO: 15 | SEQ ID NO: 16 |
| | EVQLVESGGGLVQPGGSLRLSC | DIVMTQSPSSLTVTAGEKVTM |
| | ATSGFTLSDYYMSWVRQPPGK | SCKSSQSLLNSINQKNFLTWY |
| | APEWLGFMRNKANGYTTEYSA | QQKPGQPPKLLIYWASTRESG |
| | SVRGRFTISRDTSQSIIYLQMNT | VPDRFTGSGSGTDFTLTISSVQ |
| | LRAEDGATYYCVRDRDGRPFA | AEDLAIYYCQNDYTYPLTFGT |
| | YWGQGTLVTVSA | GTKLELK |
| 18-F9-D8-G7 | SEQ ID NO: 23 | SEQ ID NO: 24 |
| | EVQLQQSGPELVRPGVSVKISC | ENVLTQSPAIMSASPGEKVTM |
| | KGSGYTFTDYAMHWVKQSHA | TCRASSSVSSSYLHWYQQKSG |
| | QSLEWIGIINTYSGDTNYNQKF | DSPKLWIYSTSNLASGVPARFS |
| | KGKATMTVDKSSSTAYMGLA | GSGSGTSYSLTISSVEAEDAAT |
| | RLTSEDSAIFYCARGGGNYPY | YYCQQFSVYPLTFGAGTKLEI |
| | WYFDVWGAGTTVTVSS | K |
| 9-G2-H6-E4 | SEQ ID NO: 31 | SEQ ID NO: 32 |
| | EVQLQQSGPDLVKPGASVKISC | DIVMSQSPSSLAVSVGEKVNM |
| | KASGYSFTGYFMNWVKQRHG | SCKSSQSLLYSRNQKNYLAWN |
| | KSLEWIGYINPNNGDTIYNQKF | QQKPGQSPKLLIYWASARESG |
| | KGKATLTVDKSSSTAHMELLS | VPDRFTGSGSGTDFTLTISSVK |
| | LTSEDSAVYYCGRGGLPFGAL | AEDLAAYYCQQYHSYPYTFG |
| | DYWGQGTSVTVSS | GGTKLELK |
| 20-F8-B5-G2 | SEQ ID NO: 39 | SEQ ID NO: 40 |
| | EVQLQESGPSLVKPSQTLSLTC | DIVMTQATPSVPVTPGESISISC |
| | SVTGDSITSAYWNWIRKFPGNK | RSSKSLLHSNGNTYLYWFLQR |
| | LEYMGYISNSGSTYYNPSLKSR | PGQSPQLLIYRMSNLASGVPD |
| | ISITRDTSKNQYYLQLNSVTTE | RFSGSGSGTAFTLRISRVEAED |
| | DTGTYYCVRGERYYGYGAYW | VGVYYCMQHLEYPFTFGAGT |
| | GQGTLVTVSA | KLELK |
| 30-C7-C11-D4 | SEQ ID NO: 47 | SEQ ID NO: 48 |
| | EVQLVESGPGLVKPSQSLSLTC | DIVMTQSPSSLAMSVGQKVTL |
| | TVTGYSITSDYAWNWIRQFPG | SCKSSQSLLNSSTQKNYLAWY |
| | NKLEWMGYISHSGSTSYNPSLK | QQKPGQSPKLLVYFASTRDSG |
| | SRISITRDTSKNQFFLQLNSVTT | VPDRFIGSGSGTEFTLTISSVQA |
| | EDTATYYCARSLGRRWYFDV | EDLADYFCQQHYIIPFTFGAGT |
| | WGAGTTVTVSS | KLEIK |
| 23-F10-G4- | SEQ ID NO: 55 | SEQ ID NO: 56 |
| F11 | EVQLQESGPDLVRPGALVKLSC | QIVLTQSPEIMSASPGEKVTMT |
| | KSSGFNIKDYPIHWVRQRPEQG | CSASSSVSYIHWYQQKSGTSP |
| | LEWIGWFDPDNGNVIYDPRFQ | KRWIYDTSKLASGVPARFSGS |
| | GKASITADTSSNTAYLQLNSLT | GSGTSYSLTIRSMEAEDAATY |
| | SEDTAVYYCSSPYGAMDYWG | YCQQGSSNPRTFGGGTKLEIK |
| | QGTSVTVSS | |
| 6-H11-G5-D8 | SEQ ID NO: 63 | SEQ ID NO: 64 |
| | EVQLQESGPELVKPGTSVKLSC | QIVLTQSPEIMSASPGEKVTMT |
| | KASGYTFTSYDINWVRQRPEQ | CSASSSVSYIHWYQQKSGTSP |
| | GLEWIGWIFPGNGDTYYNEKF | KRWIYDTSKLASGVPARFSGS |
| | KGKATLTSDKSSSTAYMQLSR | GSGTSYSLTIRSMEAEDAATY |
| | LTSEDSAVYFCARDLRLGTFDY | YCQQGSSNPRTFGGGTKLELK |
| | WGQGTTLTVSS | |
| 27-E7-D8-C7 | SEQ ID NO: 71 | SEQ ID NO: 72 |
| | EVQLQESGPELVRPGVSVKISC | QIVLSQSPAILSASPGEKVTMT |
| | KGSGYTFTDYAMHWVKQSHA | CRASSSVSYMHWYQQKPGSSP |
| | KSLEWIGVINTYSDNTNYNRKF | KPWIYATSNLASGVPARFSGS |
| | KGKATMTVDKSSSTAYMELAR | GSGTSYSLTISRVEAEDAATYY |
| | LTSEDSAIYYCARGLLLRSQLG | CQQWSSNPWTFGGGTKLEIK |
| | VYFDYWGQGTTLTVSS | |
| 30-E2-G7-G7 | SEQ ID NO: 79 | SEQ ID NO: 80 |
| | EVQLQESGGGLVKPGGSLKLS | DIVMTQSQKFMSTSVGDRVSV |
| | CAASGFTFSYYAMSWVRQTPE | TCKASQNVGTNVAWYHQRPG |
| | KRLEWVATINGDGSHTYYPDS | QSPKPLIYSASYRYSGVPDRFT |
| | VRGRFTISRDNAKNTLYLQVSS | GSGSGTDFTLTISNVQSEDLAE |
| | LRSEDTAMYYCASLLQLGPFW | YFCQQYNNYPLTFGSGTKLEI |
| | GQGTTLTVSS | K |
| 5-D1-G6-D9 | SEQ ID NO: 87 | SEQ ID NO: 88 |
| | EVQLQESGAELVKPGASVKLS | DIQMTQTTSSLSASLGDRVTIS |
| | CKTSGLTFSTNYISWLKQKPGQ | CRASQDISNYLNWYQQKPDGT |
| | SLEWIAWIYGGNGGTSYNQRF | VKLLIYFTSRLHSGVPSRFSAS |
| | TGKAQLTVDTSSSTAYMQFSSL | GSGTDYSLTISNLEQEDIATYF |
| | TTEDSAIYYCTSHDYYGGPMD | CQQTNTLPWTFGGGTKLEIK |
| | YWGQGTSVTVSS | |
| 3-C2-C3-E7 | SEQ ID NO: 95 | SEQ ID NO: 96 |
| | EVQLQQSGPELVKPGASVKISC | QIVLTQSPAIMSASPGEKVTMT |
| | KTSGYTFTENTMHWVKQSHG | CSASSSVSYMHWYQQKSGTSP |
| | KRLEWIGGINPNTGGTTYNQKF | KRWIYDTSKLASGVPARFSGS |
| | KGKATLTVDKSSSTAYMELRS | GSGTSYSLTISSMEAEDAATYY |
| | LTSEDSAVYYCARRGTNWEH | CQQWSSNPLTFGAGTKLELK |
| | WYFDVWGAGTTVTVSS | |
| 11-G10-B4- | SEQ ID NO: 103 | SEQ ID NO: 104 |
| B11 | DVQLQESGPGLVKPSQSLSLTC | DVVMTQTPLSLPVSLGDQASIS |
| | TVTGYSITSDYAWNWIRQFPG | CRSSQSLVHSNGNTYLHWYLQ |
| | NKLEWMGYISYSGSTSYNPSLK | KPGQSPKLLIYKVSNRFSGVPD |
| | SRISITRDTSKNQFFLQLNSVTT | RFSGSGSGTDFTLKISRVEAED |
| | EDTATYYCARGGPWFAYWGQ | LGVYFCSQSTHVPFTFGSGTKL |
| | GTLVTVSA | EIK |
| 16-C6-F7-F5 | SEQ ID NO: 111 | SEQ ID NO: 112 |
| | QIQLVQSGPEVKKPGETVKISC | DIVMTQTPLSLPVSLGDQASIS |
| | KASGYTFTKYGINWVKQAPGE | CRSSQSLLHTNGDTYLHWYLQ |
| | GLKWMGWIWTYTGKPTYGDD | KAGQPPKLLIYKVSNRFSGVP |
| | FKGRFAFSLETSASTAYLQISDL | DRFSGSGSGTDFTLKISRVEAE |
| | KIEDMATYFCARDDGYYYTM | DLGVYFCSQSTHIPWTFGGGT |
| | NYWGQGTSVTVSS | KLEIK |
| 22-E11-C3-F2 | SEQ ID NO: 119 | SEQ ID NO: 120 |
| | QIQLVQSGPELKRPGETVKISC | DVVMTQPPLSLPVSLGDQASIS |
| | KASGYSFTNYGMNWVKQAPG | CRSSQSLIHSNGDTYLHWYLQ |
| | KGLKWMGWINPYTGEPTYAD | KPGQSPKLLIYKVSIRFSGVPD |
| | DFKGRFAMSLETSASAAYLQIN | RFSGSGSGTDFTLKISRVEAED |
| | NLKNEDMATYFCGRDDGYHY | LGVYFCSQSTYVPWTFGGGTK |
| | TMNYWGQGTSVTVSS | LEIK |
| 24-C10-F9-G7 | SEQ ID NO: 127 | SEQ ID NO: 128 |
| | EVQLQESGPELVRPGVSVKISC | DIQMTQSPASLFASVGETVTIT |
| | KGSGYTFTDHTMHWVKESHA | CRASEKIYSYLAWYFQKQGKS |
| | QSLEWIGVISTYYGNTNYNQNF | PQFLVYNANALAEGVPSRFSG |
| | KGKATLTVDKSSSTAYMELAG | SGSGTQFSLKINSLQPEDFGTY |
| | LTSEDSAIYYCARASYGSGYGP | YCQHHYGTPVTFGAGSKLELK |
| | YYWYFDVWGAGTTVTVSS | |
| 25-C8-D7-C5 | SEQ ID NO: 135 | SEQ ID NO: 136 |
| | EVQLQESGGGLVKPGGSLKFSC | QIVLTQSPAIMSASPGEKVTMT |
| | AASGFTFSTYAMSWVRQTPEK | CRASSSVSFMHWYQQKSGTSP |
| | RLEWVATISGGGIDTYYPDSVK | KRWIYETSKLASGVPDRFSGS |
| | GRFTISRDNAKNTLYLQMNSLR | GSGTSYSLTISSMEAEDAATYY |
| | SEDTAMYYCARQGSKGWGAM | CQQWSSNPLTFGAGTKLELK |
| | DYWGQGTSVTVSS | |
| 4-D5-B9-B11 | SEQ ID NO: 143 | SEQ ID NO: 144 |
| | EIGGLVKPGGSLKFSCAASGFT | QIVLTQSPAIMSASPGEKVTMT |
| | FSSYAMSWVRQTPEKRLEWVA | CRASSSVSFMHWYQQKSGTSP |
| | TISGGGIYYPDSVKGRFTISRDN | KRWIYETSKLASGVPDRFSGS |
| | AKNTLYLQMSSLRSEDTAMYY | GSGTSYSLTISSMEAEDAATYY |
| | CARQGSKGWDAMDYWGQGT | CQQWSSNPLTFGAGTKLELK |
| | SVTVSS | |
| 10-B9-D10- | SEQ ID NO: 151 | SEQ ID NO: 152 |
| A12 | EVQLQESGPELVRPGVSVKISC | DIQMTQSPASLFASVGETVTIT |
| | KGSGYTFTDYTMHWVKLSHA | CRASEKIYSYLAWYFQKQGKS |
| | KSLEWIGVISTYYGNTNYNQKF | PQFLVYNANALAEGVPSRFSG |
| | KGKATLTVDKSSSTAYMELAR | SGSGTQFSLKINSLQPEDFGTY |
| | LTSEDSAIYYCARASYGSGYGP | YCQHHYGTPVTFGAGSKLELK |
| | YYWYFDVWGAGTTVTVSS | |
| 15-G1-D1-E3 | SEQ ID NO: 159 | SEQ ID NO: 160 |
| | QVQLQQPGAELVKPGTSVKLS | QLVLTQSSSASFSLGASAKLTC |
| | CKASGYTFTSYWIHWVKQRPG | TLSSQHSTYTIEWYQQQPLKPP |
| | QGLEWIGMIHPKSGTVDYNEK | KYVMELKKDGSHRTGDGIPDR |
| | FKSKATLTADKSSNTAYMQLS | FSGSSSGADRYLSISNIQPEDEA |
| | SLTSEASVVYYCARGENYDYR | IYICGVGDTIKEQFVYVFGGGT |
| | PQFVYWGQGTLVTVSA | KVTVL |
| 8-B4-F5-E11 | SEQ ID NO: 167 | SEQ ID NO: 168 |
| | QVQLQQPGAELVKPGASVKLS | QAVVTQESALTTSPGETVTLT |
| | CKASGDTFTSYWMHWVKQRP | CRSSTGAVTTSNFANWVQEKP |
| | GQGLEWIGMIHPDSGTRNYNE | DHLFTGLIGGTNNRAPGVPAR |
| | KVMSKATLNVDKSSSTAYLHL | FSGSLIGDKAALTITGAQTEDE |
| | SSLTSEDSAVYYCSRSYGSRLA | AVYFCALWYTNHFIFGSGTKV |
| | WFAYWGQGTLVTVSA | TVL |
| 6-F3-G2-G1 | SEQ ID NO: 175 | SEQ ID NO: 176 |
| | QVQLQQPGAELVKPGASVKLS | QAVVTQESALTTSPGGTVILTC |
| | CKASDYTFTNYWMHWVKQRP | RSSTGAVTTSNYANWVQEKP |
| | GQGLEWIGMIHPNSGTTSYNEK | DHLFTGLIGGTSNRAPGVPVRF |
| | FKNKATLTVDTSSATTYMHLSS | SGSLIGDKAALTITGAQTEDDA |
| | LTSEDSAVYYCASHYGNSYSA | MYFCALWYSTHYVFGGGTKV |
| | MDYWGQGTSVTVSS | TVL |
| 9-B9-H11-G7 | SEQ ID NO: 183 | SEQ ID NO: 184 |
| | DVQLQESGPGLVKPSQSLSLTC | QAVVTQESALTTSPGGTVILTC |
| | SVTGYSITSGYYWNWIRQFPGN | RSSTGAVTTSNYANWVQEKP |
| | KLEWMVYISYDGTNNYNPSLK | DHLFTGLIGGTSNRAPGVPVRF |
| | NRISITRDTSKNQFFLKLNSVTT | SGSLIGDKAALTITGAQTEDDA |
| | EDTATYYCARGDSNSYYFDYW | MYFCALWYRTHYVFGGGTKV |
| | GQGTTLTVSS | TVL |
| 9-G12-D6- | SEQ ID NO: 191 | SEQ ID NO: 192 |
| A11 | DVQLQVSGPGLVKPSQSLSLTC | QAVVTQESALTTSPGGTVILTC |
| | SVAGYSITSDYYWNWIRQFPG | RSSTGAVTTSNYANWVQEKP |
| | NKLEWMGYISYDGTNNYNPSL | DHLFTGLIGGTSNRAPGVPVRF |
| | KNRISITRDTSKGQFFLKFNSVT | SGSLIGDKAALTITGAQTEDDA |
| | TEDTATYYCARGKDGSYYFDY | MYFCALWYRTHYVFGGGTM |
| | WGQGTTLTVSS | VTVL |
| 13-A8-C4-G1 | SEQ ID NO: 199 | SEQ ID NO: 200 |
| | QVQLQQPGAELVKPGASMKLS | QAVVTQESALTTSPGETVTLT |
| | CKASGYIFTSNWMHWVKQRP | CRSSTGAVTTSNYANWVQEKP |
| | GQGLEWIGMIHPNSGTTSYNEK | DHLFTGLIGGTNNRAPGVPAR |
| | FKNKAKLTVDKSSSTAYVQLSS | FSGSLIGDKAALTITGAQTEDE |
| | LTSEDSAVYYCAGPTTTRRFYP | AIYFCALWYSNHFIFGSGTKVT |
| | MDYWGQGTSVTVSS | VL |
| 15-C8-B5-G7 | SEQ ID NO: 207 | SEQ ID NO: 208 |
| | QVQLQQPGAELVKPGASVKLS | QAVVTQESALTTSPGETVTLT |
| | CKASDYTFTSHWMHWVKQRP | CRSSTGAVTTSNYANWVQEKP |
| | GQGLEWIGMIHPNSGTTHYNE | AHLFTGLIGGTNNRAPGVPAR |
| | NFKNKATLTVDNSSSTAYMQL | FSGSLIGDKAALTITGAQTEDE |
| | SSLTSEDSAVYYCAWGSSNSYF | AIYFCALWFSNHLVFGGGTKL |
| | YAMAYWGQGTSVTVSS | TVL |
| 28-G2-E6-B10 | SEQ ID NO: 215 | SEQ ID NO: 216 |
| | QVQLQQPGAELVKPGASVKLS | QAVVTQESALTTSPGGTVILTC |
| | CKASGYTFTSYWMHWVKQRP | RSSTGAVTTSNYASWVQEKPD |
| | GQGLEWIGMIHPNSDTTDYNE | HLFTGLIGGTSNRAPGVPVRFS |
| | KFKTKATLTVDKSSSTAYMQL | GSLIGDKAALTITGAQTEDDA |
| | SSLTSEDSAVFYCARGGLGPHY | MYFCALWYSPHYVFGGGTKV |
| | FVYWGQGTTLTVSS | TVL |
| 3-G7-D8-D3 | SEQ ID NO: 223 | SEQ ID NO: 224 |
| | QVQLQQSGAELARPGTSVKLS | QIVLTQSPAIMSASPGEKVTMT |
| | CKASGYTFTTYWIQWVKQRPG | CSASSRINYMHWYQQKPGTSP |
| | QGLEWIGAIYPGDGDTRYTQK | IIWIYDTSKLASGVPARFSGSG |
| | FKGKATLTADKSSSTAYMQLS | SGTSYSLTISSMEAEDAATYYC |
| | TLASEDSAVYYCARPYDGYYI | HQRSSYPFTFGSGTKLEIK |
| | DYPMDSWGQGTSVTVSS | |
| 14-E7-G9-D4 | SEQ ID NO: 231 | SEQ ID NO: 232 |
| | EVQLQQSGPELVKPGASVKMS | QIVLSQSPAILSASPGEKVTMT |
| | CKASGYTFTNYIMHWVKQKPG | CRASSSVSYMHWYQQKPGSSP |
| | QGLEWIGYINPYNDGIKYNEKF | KPWIYATSNLASGVPARFSGS |
| | KGKATLTSDKSSSTAYMELSSL | GSGTSYSLTISRMEAEDAATY |
| | TSEDSAVYYCTRWGNYVFYYT | YCQQWSGNPPLTFGAGTKLEL |
| | MDYWGQGTSVTVSS | K |
| 20-C5-D7-D3 | SEQ ID NO: 239 | SEQ ID NO: 240 |
| | EVQLVESGGDLVKPGGSLKLS | DIQMTQSPASLSASVGETVTIT |
| | CAASGFTFRSYGMSWVRQTPD | CRASENIYSYLAWYQQKQGKS |
| | KRLEWVATISGGGNYTYYPDS | PQLLVYNAKTLAEGVPSRFSG |
| | VKGRFTISRDNAKNTLYLQMSS | SGSGTQFSLKINSLQPEDFGNF |
| | LKSEDTAMYYCARHEGLPLDY | YCQHHYGTPTWTFGGGTKLEI |
| | WGQGTTLTVSS | K |
| 8-C3-E3-F3 | SEQ ID NO: 247 | SEQ ID NO: 248 |
| | EVQLVESGGGLVQPGGSLKLS | DIVMTQAAFSNPVTLGTSASIS |
| | CAASGFTFSNYGLSWVRLTPD | CRSSKSLLHSNGITYLYWYLQ |
| | KRLEVVATIGSYGGNTYYPDS | KPGQSPQLLIYQMSNLASGVP |
| | VKGRFTISRDNAKNTLYLQMSS | DRESSSGSETDFTLRISRVEAE |
| | LKSEDTAIYYCARDIYYGNSEA | DVGVYYCAQNLELPWTFGGG |
| | WFAYWGQGTLVTVSA | TKLEIK |
| 2-A7-B10-A3 | SEQ ID NO: 255 | SEQ ID NO: 256 |
| | QVQLQQSGAELVRPGVSVKISC | DIQMTQTTSSLSASLGDRVTIS |
| | KGSGYTFTDYAMHWVKQSHA | CRASQDISNYLNWYQQKPDGT |
| | KSLEWIGVINTNYVNTSYNQKF | VKLLIYYTSRLHSGVPSRFSGS |
| | KGKATMTVDKSSSTAFMELAR | GSGTDYSLTISNLEQEDIATYF |
| | LTSEDSAIYYCARGGKYGNYV | CQQGNTLPYTFGGGTKLEIK |
| | FFFAYWGQGTLVTVSA | |
| 26-D2-D6- | SEQ ID NO: 263 | SEQ ID NO: 264 |
| B12 | QIQLVQSGPELKKPGETVKISC | DVVMTQTPLSLPVSLGDQASIS |
| | KASGYIFRNYGMNWVKQAPG | CRSSQSLVHSNGHTYLHWYLQ |
| | KGLKWMGWINSYTGKPTYVD | KPGQSPKLLIYKVSNRFSGVPD |
| | DFKGRFAFSLETSASTAYLQIN | RFSGSGSGTDFTLKISRVEAED |
| | NLKNEDMATYFCARDDGYWY | LGVYFCSQSTHVPWTFGGGTK |
| | TMDYWGQGTSVTVSS | LEIK |
| 11-C12-F4-F6 | SEQ ID NO: 271 | SEQ ID NO: 272 |
| | QIQLVQSGPELKKPGETVKISC | DVVMTQTPPSLPVSLGDQASIP |
| | KASGYTFRNFGMNWVKQGPG | CRSSQSLVHSNGITYLHWYLQ |
| | KGLKWMGWINPYTGEPTYVG | KPGQSPKLLIYKVSNRFSGVPD |
| | DFKGRFAFSLETSASTAYLQIN | RFSGSGSGTDFTLKISRVEAED |
| | NLKNEDMATYFCARDDGYWY | LGVYFCSQSTHVPWTFGGGTK |
| | TMDYWGQGTSVTVSS | LEIK |
| 16-G3-D10- | SEQ ID NO: 279 | SEQ ID NO: 280 |
| C10 | QIQLVQSGPELKKPGETVKISC | DVVMTQTPPSLPVSLGDQASIP |
| | KASGYTFRNYGMNWVKQGPG | CRSSQSLVHSNGITYLHWYLQ |
| | KDLKWMGWINPYTGEPTYIGD | KPGQSPKLLIYKVSNRFSGVPD |
| | FKGRFAFSLETSANSAYLQINN | RFSGSGSGTDFTLKISRVEAED |
| | LNNEDMATYFCARDDGYWYT | LGVYFCSQSTHVPWTFGGGTK |
| | MDYWGQGTSVTVSS | LEIK |
| 27-F8-E10- | SEQ ID NO: 287 | SEQ ID NO: 288 |
| E11 | EVQLQQSGPELVKPGASVKISC | QIVLTQSPAIMSASPGEKVTMT |
| | KTSGYTFTEYAMHWVKQSHG | CSASSSVSYMHWYQQKSGTSP |
| | KILEWIGGINPNIGGTTYNQKF | KRWIYDTSRVASGVPARFSGS |
| | KDKATLTVDKSSSTASMELRSL | GSGTSYSLTISSMEAEDAATYY |
| | TSEDSAVYYCARFLVPSTEGFT | CQQWSSNPLTFGAGTKLELK |
| | YWGQGTLVTVSA | |
| 7-E1-F8-F6 | SEQ ID NO: 295 | SEQ ID NO: 296 |
| | QVQLQQSGPELVKPGASVQMS | DVVMTQTPLSLPVSLGDQASIS |
| | CKASGYTFTDYVISWVNQRTG | CRSTQSLVHSNGVTYLHWYL |
| | QGLEWIGEIYPRSGNTYYNEKF | QKPGQSPKLLIYKVSNRFSGVP |
| | KGKATLTADKSSNTAYMQLSS | DRFSGSGSGTDFTLKISRVEAE |
| | LTSEDSAVYFCARFDIAYSFDY | DLGVYFCSQGTHVPWTFGGG |
| | WGQGTTLTVSS | TKLEIK |
| 25-D3-G4-C6 | SEQ ID NO: 303 | SEQ ID NO: 304 |
| | EIQLQQTGPELVKPGASVKISC | DVVMTQTPLSLPVSLGDQASIS |
| | KASGYSFTDYIILWVKQSHGMS | CRSSQSLVHSNGNTYLHWYLQ |
| | LEWIGNINPYYNSTYYNLKFKG | KPGQSPKLLIYKVSNRFSGVPD |
| | KATLTVDKSSSTAYMQLDSLTS | RFSGSGSGTDFTLKISRVETED |
| | EDSAVYYCARDSFDWYFGVW | LGIYFCSQSTHVPPLTFGAGTK |
| | GAGTTVTVSS | LELK |
| 13-E4-G9-A4 | SEQ ID NO: 311 | SEQ ID NO: 312 |
| | EVMLVESGGGLVKPGGSLKFS | QIVLTQSPPIMSASPGERVTMT |
| | CAASGFTFITYAMSWVRQTPE | CRASSRVSFMHWYQQKSGTSP |
| | KRLEWVATISGGGIDTYYPDSV | RRWIYETSKLASGVPDRFSGS |
| | KGRFTISRDNAKNTLYLQVNSL | VSGTSYSLTISSMEAEDAATYY |
| | RSEDTAMYYCTRQGSQGWGA | CQQWSSNPLTFGAGTKLELK |
| | MDYWGQGTSVTVSS | |
| 20-A2-D10- | SEQ ID NO: 319 | SEQ ID NO: 320 |
| G8 | QVQLQQSGPELVRPGVSVKISC | DIQMTQSPASLFASVGETVTIT |
| | KGSGYTFTDYTMHWVKESHA | CRASEKIYSYLAWYFQKQGKS |
| | QSLEWIGVISTYYGNTNYNQNF | PQFLVYNANALAEGVPSRFSG |
| | KGKATLTVDKSSSTAYMELAG | SGSGTQFSLKIKSLQPEDFGTY |
| | LTSEDSAIYYCARASYGSGYGP | YCQHHYGSPVTFGAGSKLELK |
| | YYWYFDVWGAGTTVTVSS | |
| 3-F2-E7-F9 | SEQ ID NO: 327 | SEQ ID NO: 328 |
| | QVQLQQSGPELVRPGVSVKISC | DIQMTQSPASLFASVGETVTIT |
| | KGSGYTFTDYTMHWVKESHA | CRASEKIYSYLAWYFQKQGKS |
| | KSLEWIGVISTYYGNTNYNQKF | PQFLVYNANALAEGVPSRFSG |
| | KGKATLTVDKSSSTAYMELAG | SGSGTQFSLKINSLQPEDFGTY |
| | LTSEDSAIYYCARASYGSGYGP | YCQHHYGTPVTFGAGSKLELK |
| | YYWYFDVWGAGTTVTVSS | |
| 6-D8-E7-A11 | SEQ ID NO: 335 | SEQ ID NO: 336 |
| | QVHLQQPGAELVKPGASVKLS | QAVVTQESALTTSPGGTVILTC |
| | CKASGYTFTNYWMHWVKRRP | RSSTGAVTTSNFANWVQEKPD |
| | GQGLEWIGMIHPNSGTTSFNEK | HLFTGLIGGTSNRAPGVPVRFS |
| | FKSKATLTVDKSSSTAYMQLSS | GSLIGDKAALTITGAQTEDDA |
| | LTSEDSAVYYCAGYYGSSFSW | MYFCALWYSTHYVFGGGTKV |
| | FTYWGQGTLVTVSA | TVL |
| 21-B3-B1-H5 | SEQ ID NO: 343 | SEQ ID NO: 344 |
| | QIQLVQSGPELKKPGETVKISC | DVVMTQTPLSLPVSLGDQASIS |
| | KASGYIFRNYGMNWVKQAPG | CRSSQSLVHSNGHTYLHWYLQ |
| | KGLKWMGWINSYTGKPTYVD | KPGQSPKLLIYKVSNRFSGVPD |
| | DFKGRFAFSLETSASTAYLQIN | RFSGSGSGTDFTLKISRVEAED |
| | NLKNEDMATYFCARDDGYWY | LGVYFCSQSTHVPWTFGGGTK |
| | TMDYWGQGTSVTVSS | LEIK |

CDRs are known to be responsible for antigen binding, however, it has been found that not all of the 6 CDRs are indispensable or unchangeable. In other words, it is possible to replace or change or modify one or more CDRs in anti-CD276 antibody 9-E8-F9-C10, 10-G6-C4-B2, 18-F9-D8-G7, 9-G2-H6-E4, 20-F8-B5-G2, 30-C7-C11-D4, 23-F10-G4-F11, 6-H11-G5-D8, 27-E7-D8-C7, 30-E2-G7-G7, 5-D1-G6-D9, 3-C2-C3-E7, 11-G10-B4-B11, 16-C6-F7-F5, 22-E11-C3-F2, 24-C10-F9-G7, 25-C8-D7-C5, 4-D5-B9-B11, 10-B9-D10-A12, 15-G1-D1-E3, 8-B4-F5-E11, 6-F3-G2-G1, 9-B9-H11-G7, 9-G12-D6-A11, 13-A8-C4-G1, 15-C8-B5-G7, 28-G2-E6-B10, 3-G7-D8-D3, 14-E7-G9-D4, 20-C5-D7-D3, 8-C3-E3-F3, 2-A7-B10-A3, 26-D2-D6-B12, 11-C12-F4-F6, 16-G3-D10-C10, 27-F8-E10-E11, 7-E1-F8-F6, 25-D3-G4-C6, 13-E4-G9-A4, 20-A2-D10-G8, 3-F2-E7-F9, 6-D8-E7-A11, and 21-B3-B1-H5, yet substantially retain the specific binding affinity to CD276.

In certain embodiments, the anti-CD276 antibodies and the antigen-binding fragments provided herein comprise a heavy chain CDR3 sequence of one of the anti-CD276 antibodies 9-E8-F9-C10, 10-G6-C4-B2, 18-F9-D8-G7, 9-G2-H6-E4, 20-F8-B5-G2, 30-C7-C11-D4, 23-F10-G4-F11, 6-H11-G5-D8, 27-E7-D8-C7, 30-E2-G7-G7, 5-D1-G6-D9, 3-C2-C3-E7, 11-G10-B4-B11, 16-C6-F7-F5, 22-E11-C3-F2, 24-C10-F9-G7, 25-C8-D7-C5, 4-D5-B9-B11, 10-B9-D10-A12, 15-G1-D1-E3, 8-B4-F5-E11, 6-F3-G2-G1, 9-B9-H11-G7, 9-G12-D6-A11, 13-A8-C4-G1, 15-C8-B5-G7, 28-G2-E6-B10, 3-G7-D8-D3, 14-E7-G9-D4, 20-C5-D7-D3, 8-C3-E3-F3, 2-A7-B10-A3, 26-D2-D6-B12, 11-C12-F4-F6, 16-G3-D10-C10, 27-F8-E10-E11, 7-E1-F8-F6, 25-D3-G4-C6, 13-E4-G9-A4, 20-A2-D10-G8, 3-F2-E7-F9, 6-D8-E7-A11, and 21-B3-B1-H5. In certain embodiments, the anti-CD276 antibodies and the antigen-binding fragments provided herein comprise a heavy chain CDR3 sequence selected from the group consisting of SEQ ID NOs: 3, 11, 19, 27, 35, 43, 51, 59, 67, 75, 83, 91, 99, 107, 115, 123, 131, 139, 147, 155, 163, 171, 179, 187, 195, 203, 211, 219, 227, 235, 243, 251, 259, 267, 275, 283, 291, 299, 307, 315, 323, 331, and 339. Heavy chain CDR3 regions are located at the center of the antigen-binding site, and therefore are believed to make the most contact with antigen and provide the most free energy to the affinity of antibody to antigen. It is also believed that the heavy chain CDR3 is by far the most diverse CDR of the antigen-binding site in terms of length, amino acid composition and conformation by multiple diversification mechanisms (Tonegawa S. Nature. 302:575-81). The diversity in the heavy chain CDR3 is sufficient to produce most antibody specificities (Xu J L, Davis M M. Immunity. 13:37-45) as well as desirable antigen-binding affinity (Schier R, etc. J Mol Biol. 263:551-67).

In certain embodiments, the antibodies and/or antigen-binding fragments thereof provided herein comprise suitable framework region (FR) sequences, as long as the antibodies and/or antigen-binding fragments thereof can specifically bind to CD276. The CDR sequences provided in Table 1 are obtained from mouse antibodies, but they can be grafted to any suitable FR sequences of any suitable species such as mouse, human, rat, rabbit, among others, using suitable methods known in the art such as recombinant techniques.

In certain embodiments, the antibodies and/or antigen-binding fragments thereof provided herein are PTM optimized. As used herein, PTM optimization refers to post-translation modification, aiming at avoiding potential aggregation, activity loss or other risk. Exemplary PTM optimized antibody and/or antigen-binding fragments thereof includes mVH5-mVL4-10 and mVH-mVL1-30, derived from 10-G6-C4-B2 and 30-C7-C11-D4, respectively. The variable region sequences of mVH5-mVL4-10 and mVH-mVL1-30 are shown in Table 3, wherein all the CDR regions are underlined.

TABLE 3
CDR sequences and variable region sequences of the anti-CD276
antibodies with PTM optimization

| Clone | VH | | VL | |
|---|---|---|---|---|
| mVH5-mVL4- | SEQ ID NO: 347 | | SEQ ID NO: 348 | |
| 10 | EVQLVESGGGLVQPGGSLRLSC | | DIVMTQSPSSLTVTAGEKVTM | |
| | ATSGFTLSDYYMSWVRQPPGK | | SCKSSQSLLNAINQKNFLTWY | |
| | APEWLGFMRNKANAYTTEYSA | | QQKPGQPPKLLIYWASTRESG | |
| | SVRGRFTISRDTSQSIIYLQMNT | | VPDRFTGSGSGTDFTLTISSVQ | |
| | LRAEDGATYYCVRDREGRPFA | | AEDLAIYYCQNDYTYPLTFGT | |
| | YWGQGTLVTVSA | | GTKLELK | |
| | CDR1 | SEQ ID NO: 9 | CDR1 | SEQ ID NO: 376 |
| | | GFTLSDYY | | QSLLNAINQKNF |
| | CDR2 | SEQ ID NO: 374 | CDR2 | SEQ ID NO: 13 |
| | | MRNKANAYTT | | WAS |
| | CDR3 | SEQ ID NO: 375 | CDR3 | SEQ ID NO: 14 |
| | | VRDREGRPFAY | | QNDYTYPLT |
| mVH-mVL1- | SEQ ID NO: 349 | | SEQ ID NO: 350 | |
| 30 | EVQLVESGPGLVKPSQSLSLTC | | DIVMTQSPSSLAMSVGQKVTL | |
| | TVTGYSITSDYAWNWIRQFPG | | SCKSSQSLLQSSTQKNYLAWY | |
| | NKLEWMGYISHSGSTSYNPSLK | | QQKPGQSPKLLVYFASTRDSG | |
| | SRISITRDTSKNQFFLQLNSVTT | | VPDRFIGSGSGTEFTLTISSVQA | |
| | EDTATYYCARSLGRRWYFDV | | EDLADYFCQQHYIIPFTFGAGT | |
| | WGAGTTVTVSS | | KLEIK | |
| | CDR1 | SEQ ID NO: 41 | CDR1 | SEQ ID NO: 377 |
| | | GYSITSDYA | | QSLLQSSTQKNY |
| | CDR2 | SEQ ID NO: 42 | CDR2 | SEQ ID NO: 45 |
| | | SHSGST | | FAS |
| | CDR3 | SEQ ID NO: 43 | CDR3 | SEQ ID NO: 46 |
| | | ARSLGRRWYFDV | | QQHYIIPFT |

In certain embodiments, the antibodies and/or antigen-binding fragments thereof provided herein are humanized. A humanized antibody or antigen-binding fragment is desirable in its reduced immunogenicity in human. A humanized antibody is chimeric in its variable regions, as non-human CDR sequences are grafted to human or substantially human FR sequences. Humanization of an antibody or antigen-binding fragment can be essentially performed by substituting the non-human (such as murine) CDR genes for the corresponding human CDR genes in a human immunoglobulin gene (see, for example, Jones et al. (1986) Nature 321:522-525; Riechmann et al. (1988) Nature 332:323-327; Verhoeyen et al. (1988) Science 239:1534-1536).

Suitable human heavy chain and light chain variable domains can be selected to achieve this purpose using methods known in the art. In an illustrative example, “best-fit” approach can be used, where a non-human (e.g. rodent) antibody variable domain sequence is screened or BLASTed against a database of known human variable domain sequences, and the human sequence closest to the non-human query sequence is identified and used as the human scaffold for grafting the non-human CDR sequences (see, for example, Sims et al, (1993) J. Immunol. 151:2296; Chothia et al. (1987) J. Mot. Biol. 196:901). Alternatively, a framework derived from the consensus sequence of all human antibodies may be used for the grafting of the non-human CDRs (see, for example, Carter et al. (1992) Proc. Natl. Acad. Sci. USA, 89:4285; Presta et al. (1993) J. Immunol., 151:2623). In certain embodiments, the humanized antibodies or antigen-binding fragments provided herein are composed of substantially all human sequences except for the CDR sequences which are non-human. In some embodiments, the variable region FRs, and constant regions if present, are entirely or substantially from human immunoglobulin sequences. The human FR sequences and human constant region sequences may be derived different human immunoglobulin genes, for example, FR sequences derived from one human antibody and constant region from another human antibody.

Table 4 below shows the heavy chain and light chain variable region amino acid sequences of humanized antibodies for 10-G6-C4-B2 and 30-C7-C11-D4, which are designated as hVH2-hVL1-10, hVH3-hVL3-10, hVH4-hVL1-10, hVH4-hVL2-10, hVH5-hVL1-10, hVH5-hVL3-10, 10-G6-C4-B2_hVH2-VL1_PTM, 10-G6-C4-B2_hVH3-VL3_PTM, 10-G6-C4-B2_hVH4-VL1_PTM, 10-G6-C4-B2_hVH4-VL2_PTM, 10-G6-C4-B2_hVH5-VL1_PTM, 10-G6-C4-B2 hVH5-VL3_PTM, hVH1-hVL5-30, hVH2-hVL1-30, hVH3-hVL2-30, hVH3-hVL5-30, hVH4-hVL2-30, hVH4-hVL5-30, 30-C7-C11-D4_hVH1-hVL5_PTM, 30-C7-C11-D4_hVH2-hVL1_PTM, 30-C7-C11-D4_hVH3-hVL2_PTM, 30-C7-C11-D4_hVH3-hVL5_PTM, 30-C7-C11-D4_hVH4-hVL2_PTM and 30-C7-C11-D4 hVH4-hVL5 PTM, wherein all the CDR regions are underlined.

TABLE 4
Variable region amino acid sequences of the umanized
antibodies

| Antibody | VH | VL |
|---|---|---|
| hVH2- | SEQ ID NO: 351 | SEQ ID NO: 352 |
| hVL1-10 | EVQLVESGGGLVQPGGSLRLS | DIVMTQSPDSLAVSLGERATI |
| | CATSGFTLSDYYMSWVRQAP | NCKSSQSLLNSINQKNFLTW |
| | GKGLEWVSFMRNKANGYTT | YQQKPGQPPKLLIYWASTRE |
| | EYSASVRGRFTISRDTSKSTIY | SGVPDRFSGSGSGTDFTLTIS |
| | LQMNSLRAEDTAVYYCVRDR | SLQAEDLAVYYCQNDYTYP |
| | DGRPFAYWGQGTLVTVSS | LTFGQGTKLEIK |
| hVH3- | SEQ ID NO: 353 | SEQ ID NO: 354 |
| hVL3-10 | EVQLVESGGGLVQPGGSLRLS | DIQMTQSPSSLSASVGDRVTI |
| | CATSGFTLSDYYMSWVRQAP | TCKSSQSLLNSINQKNFLTW |
| | GKGLEWVGFMRNKANGYTT | YQQKPGKAPKLLIYWASTRE |
| | EYSASVRGRFTISRDTSKSSIY | SGVPSRFSGSGSGTDFTLTIS |
| | LQMNSLKTEDTAVYYCVRDR | SLQPEDLATYYCQNDYTYPL |
| | DGRPFAYWGQGTLVTVSS | TFGQGTKLEIK |
| hVH4- | SEQ ID NO: 355 | SEQ ID NO: 352 |
| hVL1-10 | EVQLVESGGGLIQPGGSLRLS | DIVMTQSPDSLAVSLGERATI |
| | CATSGFTLSDYYMSWVRQPP | NCKSSQSLLNSINQKNFLTW |
| | GKGLEWVSFMRNKANGYTT | YQQKPGQPPKLLIYWASTRE |
| | EYSASVRGRFTISRDTSKSTIY | SGVPDRFSGSGSGTDFTLTIS |
| | LQMNSLRAEDTAVYYCVRDR | SLQAEDLAVYYCQNDYTYP |
| | DGRPFAYWGQGTLVTVSS | LTFGQGTKLEIK |
| hVH4- | SEQ ID NO: 355 | SEQ ID NO: 356 |
| hVL2-10 | EVQLVESGGGLIQPGGSLRLS | DIVMTQSPLSLPVTPGEPASI |
| | CATSGFTLSDYYMSWVRQPP | SCKSSQSLLNSINQKNFLTW |
| | GKGLEWVSFMRNKANGYTT | YLQKPGQSPQLLIYWASTRE |
| | EYSASVRGRFTISRDTSKSTIY | SGVPDRFSGSGSGTDFTLKIS |
| | LQMNSLRAEDTAVYYCVRDR | RVEAEDLGVYYCQNDYTYP |
| | DGRPFAYWGQGTLVTVSS | LTFGQGTKLEIK |
| hVH5- | SEQ ID NO: 357 | SEQ ID NO: 352 |
| hVL1-10 | EVQLVESGGGLVQPGRSLRLS | DIVMTQSPDSLAVSLGERATI |
| | CTTSGFTLSDYYMSWVRQAP | NCKSSQSLLNSINQKNFLTW |
| | GKGLEWVGFMRNKANGYTT | YQQKPGQPPKLLIYWASTRE |
| | EYSASVRGRFTISRDTSKSIIYL | SGVPDRFSGSGSGTDFTLTIS |
| | QMNSLKTEDTAVYYCVRDRD | SLQAEDLAVYYCQNDYTYP |
| | GRPFAYWGQGTLVTVSS | LTFGQGTKLEIK |
| hVH5- | SEQ ID NO: 357 | SEQ ID NO: 354 |
| hVL3-10 | EVQLVESGGGLVQPGRSLRLS | DIQMTQSPSSLSASVGDRVTI |
| | CTTSGFTLSDYYMSWVRQAP | TCKSSQSLLNSINQKNFLTW |
| | GKGLEWVGFMRNKANGYTT | YQQKPGKAPKLLIYWASTRE |
| | EYSASVRGRFTISRDTSKSIIYL | SGVPSRFSGSGSGTDFTLTIS |
| | QMNSLKTEDTAVYYCVRDRD | SLQPEDLATYYCQNDYTYPL |
| | GRPFAYWGQGTLVTVSS | TFGQGTKLEIK |
| 10-G6-C4- | SEQ ID NO: 358 | SEQ ID NO: 359 |
| B2_hVH2- | EVQLVESGGGLVQPGGSLRLS | DIVMTQSPDSLAVSLGERATI |
| VL1_PTM | CATSGFTLSDYYMSWVRQAP | NCKSSQSLLNAINQKNFLTW |
| | GKGLEWVSFMRNKANAYTT | YQQKPGQPPKLLIYWASTRE |
| | EYSASVRGRFTISRDTSKSTIY | SGVPDRFSGSGSGTDFTLTIS |
| | LQMNSLRAEDTAVYYCVRDR | SLQAEDLAVYYCQNDYTYP |
| | EGRPFAYWGQGTLVTVSS | LTFGQGTKLEIK |
| 10-G6-C4- | SEQ ID NO: 360 | SEQ ID NO: 361 |
| B2_hVH3- | EVQLVESGGGLVQPGGSLRLS | DIQMTQSPSSLSASVGDRVTI |
| VL3_PTM | CATSGFTLSDYYMSWVRQAP | TCKSSQSLLNAINQKNFLTW |
| | GKGLEWVGFMRNKANAYTT | YQQKPGKAPKLLIYWASTRE |
| | EYSASVRGRFTISRDTSKSSIY | SGVPSRFSGSGSGTDFTLTIS |
| | LQMNSLKTEDTAVYYCVRDR | SLQPEDLATYYCQNDYTYPL |
| | EGRPFAYWGQGTLVTVSS | TFGQGTKLEIK |
| 10-G6-C4- | SEQ ID NO: 362 | SEQ ID NO: 359 |
| B2_hVH4- | EVQLVESGGGLIQPGGSLRLS | DIVMTQSPDSLAVSLGERATI |
| VL1_PTM | CATSGFTLSDYYMSWVRQPP | NCKSSQSLLNAINQKNFLTW |
| | GKGLEWVSFMRNKANAYTT | YQQKPGQPPKLLIYWASTRE |
| | EYSASVRGRFTISRDTSKSTIY | SGVPDRFSGSGSGTDFTLTIS |
| | LQMNSLRAEDTAVYYCVRDR | SLQAEDLAVYYCQNDYTYP |
| | EGRPFAYWGQGTLVTVSS | LTFGQGTKLEIK |
| 10-G6-C4- | SEQ ID NO: 362 | SEQ ID NO: 363 |
| B2_hVH4- | EVQLVESGGGLIQPGGSLRLS | DIVMTQSPLSLPVTPGEPASI |
| VL2_PTM | CATSGFTLSDYYMSWVRQPP | SCKSSQSLLNAINQKNFLTW |
| | GKGLEWVSFMRNKANAYTT | YLQKPGQSPQLLIYWASTRE |
| | EYSASVRGRFTISRDTSKSTIY | SGVPDRFSGSGSGTDFTLKIS |
| | LQMNSLRAEDTAVYYCVRDR | RVEAEDLGVYYCQNDYTYP |
| | EGRPFAYWGQGTLVTVSS | LTFGQGTKLEIK |
| 10-G6-C4- | SEQ ID NO: 364 | SEQ ID NO: 359 |
| B2_hVH5- | EVQLVESGGGLVQPGRSLRLS | DIVMTQSPDSLAVSLGERATI |
| VL1_PTM | CTTSGFTLSDYYMSWVRQAP | NCKSSQSLLNAINQKNFLTW |
| | GKGLEWVGFMRNKANAYTT | YQQKPGQPPKLLIYWASTRE |
| | EYSASVRGRFTISRDTSKSIIYL | SGVPDRFSGSGSGTDFTLTIS |
| | QMNSLKTEDTAVYYCVRDRE | SLQAEDLAVYYCQNDYTYP |
| | GRPFAYWGQGTLVTVSS | LTFGQGTKLEIK |
| 10-G6-C4- | SEQ ID NO: 364 | SEQ ID NO: 361 |
| B2_hVH5- | EVQLVESGGGLVQPGRSLRLS | DIQMTQSPSSLSASVGDRVTI |
| VL3_PTM | CTTSGFTLSDYYMSWVRQAP | TCKSSQSLLNAINQKNFLTW |
| | GKGLEWVGFMRNKANAYTT | YQQKPGKAPKLLIYWASTRE |
| | EYSASVRGRFTISRDTSKSIIYL | SGVPSRFSGSGSGTDFTLTIS |
| | QMNSLKTEDTAVYYCVRDRE | SLQPEDLATYYCQNDYTYPL |
| | GRPFAYWGQGTLVTVSS | TFGQGTKLEIK |
| hVH1- | SEQ ID NO: 365 | SEQ ID NO: 366 |
| hVL5-30 | QVQLQESGPGLVKPSQTLSLT | DIVMTQSPLSLPVTPGEPASI |
| | CTVSGYSITSDYAWNWIRQHP | SCKSSQSLLNSSTQKNYLAW |
| | GKGLEWIGYISHSGSTSYNPS | YLQKPGQSPQLLIYFASTRDS |
| | LKSRVTISRDTSKNQFSLKLSS | GVPDRFSGSGSGTDFTLKISR |
| | VTAADTAVYYCARSLGRRW | VEAEDLGVYFCQQHYIIPFTF |
| | YFDVWGQGTTVTVSS | GQGTKLEIK |
| hVH2- | SEQ ID NO: 367 | SEQ ID NO: 368 |
| hVL1-30 | QVQLQESGPGLVKPSETLSLT | DIQMTQSPSSLSASVGDRVTI |
| | CTVSGYSITSDYAWNWIRQPP | TCKSSQSLLNSSTQKNYLAW |
| | GKGLEWIGYISHSGSTSYNPS | YQQKPGKAPKLLIYFASTRD |
| | LKSRVTISRDTSKNQFSLKLSS | SGVPSRFSGSGSGTDFTLTIS |
| | VTAADTAVYYCARSLGRRW | SLQPEDLATYFCQQHYIIPFT |
| | YFDVWGQGTTVTVSS | FGQGTKLEIK |
| hVH3- | SEQ ID NO: 367 | SEQ ID NO: 369 |
| hVL2-30 | QVQLQESGPGLVKPSETLSLT | DIVMTQSPDSLAVSLGERATI |
| | CTVSGYSITSDYAWNWIRQPP | NCKSSQSLLNSSTQKNYLA |
| | GKGLEWIGYISHSGSTSYNPS | WYQQKPGQPPKLLIYFASTR |
| | LKSRVTISRDTSKNQFSLKLSS | DSGVPDRFSGSGSGTDFTLTI |
| | VTAADTAVYYCARSLGRRW | SSLQAEDLAVYFCQQHYIIPF |
| | YFDVWGQGTTVTVSS | TFGQGTKLEIK |
| hVH3- | SEQ ID NO: 367 | SEQ ID NO: 366 |
| hVL5-30 | QVQLQESGPGLVKPSETLSLT | DIVMTQSPLSLPVTPGEPASI |
| | CTVSGYSITSDYAWNWIRQPP | SCKSSQSLLNSSTQKNYLAW |
| | GKGLEWIGYISHSGSTSYNPS | YLQKPGQSPQLLIYFASTRDS |
| | LKSRVTISRDTSKNQFSLKLSS | GVPDRFSGSGSGTDFTLKISR |
| | VTAADTAVYYCARSLGRRW | VEAEDLGVYFCQQHYIIPFTF |
| | YFDVWGQGTTVTVSS | GQGTKLEIK |
| hVH4- | SEQ ID NO: 370 | SEQ ID NO: 369 |
| hVL2-30 | QVQLQESGPGLVKPSQTLSLT | DIVMTQSPDSLAVSLGERATI |
| | CAVYGYSITSDYAWNWIRQP | NCKSSQSLLNSSTQKNYLA |
| | PGKGLEWIGYISHSGSTSYNPS | WYQQKPGQPPKLLIYFASTR |
| | LKSRVTISRDTSKNQFSLKLSS | DSGVPDRFSGSGSGTDFTLTI |
| | VTAADTAVYYCARSLGRRW | SSLQAEDLAVYFCQQHYIIPF |
| | YFDVWGQGTTVTVSS | TFGQGTKLEIK |
| hVH4- | SEQ ID NO: 370 | SEQ ID NO: 366 |
| hVL5-30 | QVQLQESGPGLVKPSQTLSLT | DIVMTQSPLSLPVTPGEPASI |
| | CAVYGYSITSDYAWNWIRQP | SCKSSQSLLNSSTQKNYLAW |
| | PGKGLEWIGYISHSGSTSYNPS | YLQKPGQSPQLLIYFASTRDS |
| | LKSRVTISRDTSKNQFSLKLSS | GVPDRFSGSGSGTDFTLKISR |
| | VTAADTAVYYCARSLGRRW | VEAEDLGVYFCQQHYIIPFTF |
| | YFDVWGQGTTVTVSS | GQGTKLEIK |
| 30-C7-C11- | SEQ ID NO: 365 | SEQ ID NO: 371 |
| D4_hVH1- | QVQLQESGPGLVKPSQTLSLT | DIVMTQSPLSLPVTPGEPASI |
| hVL5_PTM | CTVSGYSITSDYAWNWIRQHP | SCKSSQSLLQSSTQKNYLAW |
| | GKGLEWIGYISHSGSTSYNPS | YLQKPGQSPQLLIYFASTRDS |
| | LKSRVTISRDTSKNQFSLKLSS | GVPDRFSGSGSGTDFTLKISR |
| | VTAADTAVYYCARSLGRRW | VEAEDLGVYFCQQHYIIPFTF |
| | YFDVWGQGTTVTVSS | GQGTKLEIK |
| 30-C7-C11- | SEQ ID NO: 367 | SEQ ID NO: 372 |
| D4_hVH2- | QVQLQESGPGLVKPSETLSLT | DIQMTQSPSSLSASVGDRVTI |
| hVL1_PTM | CTVSGYSITSDYAWNWIRQPP | TCKSSQSLLQSSTQKNYLAW |
| | GKGLEWIGYISHSGSTSYNPS | YQQKPGKAPKLLIYFASTRD |
| | LKSRVTISRDTSKNQFSLKLSS | SGVPSRFSGSGSGTDFTLTIS |
| | VTAADTAVYYCARSLGRRW | SLQPEDLATYFCQQHYIIPFT |
| | YFDVWGQGTTVTVSS | FGQGTKLEIK |
| 30-C7-C11- | SEQ ID NO: 367 | SEQ ID NO: 373 |
| D4_hVH3- | QVQLQESGPGLVKPSETLSLT | DIVMTQSPDSLAVSLGERATI |
| hVL2_PTM | CTVSGYSITSDYAWNWIRQPP | NCKSSQSLLQSSTQKNYLA |
| | GKGLEWIGYISHSGSTSYNPS | WYQQKPGQPPKLLIYFASTR |
| | LKSRVTISRDTSKNQFSLKLSS | DSGVPDRFSGSGSGTDFTLTI |
| | VTAADTAVYYCARSLGRRW | SSLQAEDLAVYFCQQHYIIPF |
| | YFDVWGQGTTVTVSS | TFGQGTKLEIK |
| 30-C7-C11- | SEQ ID NO: 367 | SEQ ID NO: 371 |
| D4_hVH3- | QVQLQESGPGLVKPSETLSLT | DIVMTQSPLSLPVTPGEPASI |
| hVL5_PTM | CTVSGYSITSDYAWNWIRQPP | SCKSSQSLLQSSTQKNYLAW |
| | GKGLEWIGYISHSGSTSYNPS | YLQKPGQSPQLLIYFASTRDS |
| | LKSRVTISRDTSKNQFSLKLSS | GVPDRFSGSGSGTDFTLKISR |
| | VTAADTAVYYCARSLGRRW | VEAEDLGVYFCQQHYIIPFTF |
| | YFDVWGQGTTVTVSS | GQGTKLEIK |
| 30-C7-C11- | SEQ ID NO: 370 | SEQ ID NO: 373 |
| D4_hVH4- | QVQLQESGPGLVKPSQTLSLT | DIVMTQSPDSLAVSLGERATI |
| hVL2_PTM | CAVYGYSITSDYAWNWIRQP | NCKSSQSLLQSSTQKNYLA |
| | PGKGLEWIGYISHSGSTSYNPS | WYQQKPGQPPKLLIYFASTR |
| | LKSRVTISRDTSKNQFSLKLSS | DSGVPDRFSGSGSGTDFTLTI |
| | VTAADTAVYYCARSLGRRW | SSLQAEDLAVYFCQQHYIIPF |
| | YFDVWGQGTTVTVSS | TFGQGTKLEIK |
| 30-C7-C11- | SEQ ID NO: 370 | SEQ ID NO: 371 |
| D4_hVH4- | QVQLQESGPGLVKPSQTLSLT | DIVMTQSPLSLPVTPGEPASI |
| hVL5_PTM | CAVYGYSITSDYAWNWIRQP | SCKSSQSLLQSSTQKNYLAW |
| | PGKGLEWIGYISHSGSTSYNPS | YLQKPGQSPQLLIYFASTRDS |
| | LKSRVTISRDTSKNQFSLKLSS | GVPDRFSGSGSGTDFTLKISR |
| | VTAADTAVYYCARSLGRRW | VEAEDLGVYFCQQHYIIPFTF |
| | YFDVWGQGTTVTVSS | GQGTKLEIK |

In certain embodiments, the humanized antibodies or antigen-binding fragments thereof provided herein are composed of substantially all human sequences except for the CDR sequences which are non-human. In some embodiments, the variable region FRs, and constant regions if present, are entirely or substantially from human immunoglobulin sequences. The human FR sequences and human constant region sequences may be derived from different human immunoglobulin genes, for example, FR sequences derived from one human antibody and constant region from another human antibody. In some embodiments, the humanized antibody or antigen-binding fragment thereof comprises human heavy chain HFR1-4, and/or light chain LFR1-4.

In some embodiments, the FR regions derived from human may comprise the same amino acid sequence as the human immunoglobulin from which it is derived. In some embodiments, one or more amino acid residues of the human FR are substituted with the corresponding residues from the parent non-human antibody. This may be desirable in certain embodiments to make the humanized antibody or its fragment closely approximate the non-human parent antibody structure, so as to optimize binding characteristics (for example, increase binding affinity). In certain embodiments, the humanized antibody or antigen-binding fragment thereof provided herein comprises no more than 10, 9, 8, 7, 6, 5, 4, 3, 2, or 1 amino acid residue substitutions in each of the human FR sequences, or no more than 10, 9, 8, 7, 6, 5, 4, 3, 2, or 1 amino acid residue substitutions in all the FR sequences of a heavy or a light chain variable domain. In some embodiments, such change in amino acid residue could be present in heavy chain FR regions only, in light chain FR regions only, or in both chains. In certain embodiments, one or more amino acids of the human FR sequences are randomly mutated to increase binding affinity. In certain embodiments, one or more amino acids of the human FR sequences are back mutated to the corresponding amino acid(s) of the parent non-human antibody so as to increase binding affinity.

In certain embodiments, the anti-CD276 antibodies and the fragments thereof provided herein further comprise an immunoglobulin constant region. In some embodiments, an immunoglobulin constant region comprises a heavy chain and/or a light chain constant region. The heavy chain constant region comprises CH1, hinge, and/or CH2-CH3 regions. In certain embodiments, the heavy chain constant region comprises an Fc region. In certain embodiments, the light chain constant region comprises CK or CA.

In some embodiments, the anti-CD276 antibodies and antigen-binding fragments thereof provided herein have a constant region of an immunoglobulin (Ig), optionally a human Ig, optionally a human IgG. In some embodiments, the anti-CD276 antibodies and antigen-binding fragments thereof provided herein comprises a constant region of human IgG1, IgG2, IgG3, or IgG4.

Human IgG isotypes (the subclasses of mature gamma globulin class G antibodies; IgG1, IgG2, IgG3 and IgG4) exhibit differential capacity to recruit effector functions. For example, ADCC is promoted by IgG1 and IgG3, ADCP is promoted by IgG1, IgG2, IgG3 and IgG4, and CDC is promoted by IgG1 and IgG3. Isotype-specific engagement of such effector functions is based on selectivity for Fc receptors on distinct immune cells and the ability to bind C1q thereby activating the assembly of a membrane attack complex (MAC). Among the various isotypes, relative affinity for Fcγ receptors, which include FcγRI, FcγRIIa/b/c, and FcγRIIIa/b is high for IgG1 and IgG3. However, Fcγ affinity for IgG2 is considerably lower with the exception of FcγRIIa H131 polymorphism and IgG4 only has measurable affinity for FcγRI.

In certain embodiments, the anti-CD276 antibodies and antigen-binding fragments thereof provided herein comprises a constant region of human IgG1 isotype, which could induce ADCC, CDC or ADCP, or a constant region of IgG4 or IgG2 isotype, which has reduced or depleted effector function. Effector functions such as ADCC and CDC can lead to cytotoxicity to cells expressing CD276. Effector functions can be evaluated using various assays such as Fc receptor binding assay, C1q binding assay, and cell lysis assay.

In certain embodiments, the anti-CD276 antibodies and antigen-binding fragments thereof provided herein comprises a constant region of mouse IgG2 isotype, which could induce ADCC, CDC or ADCP.

### Antibody Variants

The present disclosure also encompasses various variants of the antibodies and/or antigen-binding fragments thereof provided herein. In certain embodiments, the present disclosure encompasses various types of variants of an exemplary antibody provided herein, i.e., the antibody with a heavy chain CDR3 sequence selected from the group consisting of SEQ ID NOs: 3, 11, 19, 27, 35, 43, 51, 59, 67, 75, 83, 91, 99, 107, 115, 123, 131, 139, 147, 155, 163, 171, 179, 187, 195, 203, 211, 219, 227, 235, 243, 251, 259, 267, 275, 283, 291, 299, 307, 315, 323, 331, 339, or 375.

In certain embodiments, the antibody variants comprise one or more modifications or substitutions in one or more CDR sequences as provided in Table 1, one or more FR sequences, the heavy or light chain variable region sequences provided in Table 2, and/or the constant region (e.g. Fc region). Such variants retain specific binding affinity to CD276 of their parent antibodies, but have one or more desirable properties conferred by the modification(s) or substitution(s). For example, the antibody variants may have improved antigen-binding affinity, improved glycosylation pattern, reduced risk of glycosylation, reduced deamination, reduced or depleted effector function(s), improved FcRn receptor binding, increased pharmacokinetic half-life, pH sensitivity, and/or compatibility to conjugation (e.g. one or more introduced cysteine residues).

The parent antibody sequence may be screened to identify suitable or preferred residues to be modified or substituted, using methods known in the art, for example “alanine scanning mutagenesis” (see, for example, Cunningham and Wells (1989) Science, 244:1081-1085). Briefly, target residues (e.g., charged residues such as Arg, Asp, His, Lys, and Glu) can be identified and replaced by a neutral or negatively charged amino acid (e.g., alanine or polyalanine), and the modified antibodies are produced and screened for the interested property. If substitution at a particular amino acid location demonstrates an interested functional change, then the position can be identified as a potential residue for modification or substitution. The potential residues may be further assessed by substituting with a different type of residue (e.g. cysteine residue, positively charged residue, etc.).

### Affinity Variant

Affinity variant may contain modifications or substitutions in one or more CDR sequences as provided in Table 1, one or more FR sequences, or the heavy or light chain variable region sequences in provided in Table 2, 3 or 4. The affinity variants retain specific binding affinity to CD276 of the parent antibody, or even have improved CD276 specific binding affinity over the parent antibody. In certain embodiments, at least one (or all) of the substitution(s) in the CDR sequences, FR sequences, or variable region sequences comprises a conservative substitution.

A skilled artisan will understand that in the CDR sequences provided in Table 1 and FR sequences, one or more amino acid residues may be substituted yet the resulting antibody or antigen-binding fragment still retain the binding affinity to CD276, or even have an improved binding affinity. Various methods known in the art can be used to achieve this purpose. For example, a library of antibody variants (such as Fab or scFv variants) can be generated and expressed with phage display technology, and then screened for the binding affinity to human CD276. For another example, computer software can be used to virtually simulate the binding of the antibodies to human CD276, and identify the amino acid residues on the antibodies which form the binding interface. Such residues may be either avoided in the substitution so as to prevent reduction in binding affinity, or targeted for substitution to provide for a stronger binding.

In certain embodiments, the humanized antibody or antigen-binding fragment provided herein comprises one or more amino acid residue substitutions in one or more CDR sequences, and/or one or more FR sequences. In certain embodiments, an affinity variant comprises no more than 10, 9, 8, 7, 6, 5, 4, 3, 2, or 1 substitutions in the CDR sequences and/or FR sequences in total.

In certain embodiments, the anti-CD276 antibodies and antigen-binding fragments thereof comprise 1, 2, or 3 CDR sequences having at least 80% (e.g. at least 85%, 88%, 90%, 91%, 92%, 93%, 94%, 95%, 96%, 97%, 98%, 99%) sequence identity to that (or those) listed in Table 1, and in the meantime retain the binding affinity to CD276 at a level similar to or even higher than its parent antibody.

In certain embodiments, the anti-CD276 antibodies and antigen-binding fragments thereof comprise one or more variable region sequences having at least 80% (e.g. at least 85%, 88%, 90%, 91%, 92%, 93%, 94%, 95%, 96%, 97%, 98%, 99%) sequence identity to that (or those) listed in Table 1, and in the meantime retain the binding affinity to CD276 at a level similar to or even higher than its parent antibody. In some embodiments, a total of 1 to 10 amino acids have been substituted, inserted, or deleted in a sequence selected from that (or those) listed in Table 1. In some embodiments, the substitutions, insertions, or deletions occur in regions outside the CDRs (i.e., in the FRs).

### Glycosylation Variant

The anti-CD276 antibodies and antigen-binding fragments provided herein also encompass a glycosylation variant, which can be obtained to either increase or decrease the extent of glycosylation of the antibody or antigen binding fragment thereof.

The antibody or antigen binding fragment thereof may comprise one or more amino acid residues with a side chain to which a carbohydrate moiety (e.g. an oligosaccharide structure) can be attached. Glycosylation of antibodies is typically either N-linked or O-linked. N-linked refers to the attachment of the carbohydrate moiety to the side chain of an asparagine residue, for example, an asparagine residue in a tripeptide sequence such as asparagine-X-serine and asparagine-X-threonine, where X is any amino acid except proline. O-linked glycosylation refers to the attachment of one of the sugars N-aceylgalactosamine, galactose, or xylose to a hydroxyamino acid, most commonly to serine or threonine. Removal of a native glycosylation site can be conveniently accomplished, for example, by altering the amino acid sequence such that one of the above-described tripeptide sequences (for N-linked glycosylation sites) or serine or threonine residues (for O-linked glycosylation sites) present in the sequence is substituted. A new glycosylation site can be created in a similar way by introducing such a tripeptide sequence or serine or threonine residue.

### Cysteine-Engineered Variant

The anti-CD276 antibodies and antigen-binding fragments provided herein also encompass a cysteine-engineered variant, which comprises one or more introduced free cysteine amino acid residues.

A free cysteine residue is one which is not part of a disulfide bridge. A cysteine-engineered variant is useful for conjugation with for example, a cytotoxic and/or imaging compound, a label, or a radioisoptype among others, at the site of the engineered cysteine, through for example a maleimide or haloacetyl. Methods for engineering antibodies or antigen-binding fragments to introduce free cysteine residues are known in the art, see, for example, WO2006/034488.

### Fc Variant

The anti-CD276 antibodies and antigen-binding fragments provided herein also encompass an Fc variant, which comprises one or more amino acid residue modifications or substitutions at its Fc region and/or hinge region.

In certain embodiments, the anti-CD276 antibodies or antigen-binding fragments comprise one or more amino acid substitution(s) that improves pH-dependent binding to neonatal Fc receptor (FcRn). Such a variant can have an extended pharmacokinetic half-life, as it binds to FcRn at acidic pH which allows it to escape from degradation in the lysosome and then be translocated and released out of the cell. Methods of engineering an antibody and antigen-binding fragment thereof to improve binding affinity with FcRn are well-known in the art, see, for example, Vaughn, D. et al, Structure, 6 (1): 63-73, 1998; Kontermann, R. et al, Antibody Engineering, Volume 1, Chapter 27: Engineering of the Fc region for improved P K, published by Springer, 2010; Yeung, Y. et al, Cancer Research, 70:3269-3277 (2010); and Hinton, P. et al, J. Immunology, 176:346-356 (2006).

In certain embodiments, the anti-CD276 antibodies or antigen-binding fragments comprise one or more amino acid substitution(s) that alters the antibody-dependent cellular cytotoxicity (ADCC). Certain amino acid residues at CH2 domain of the Fc region can be substituted to provide for enhanced ADCC activity. Alternatively or additionally, carbohydrate structures on the antibody can be changed to enhance ADCC activity. Methods of altering ADCC activity by antibody engineering have been described in the art, see for example, Shields R L. et al., J Biol Chem. 2001. 276 (9): 6591-604; Idusogie E E. et al., J Immunol. 2000.164 (8): 4178-84; Steurer W. et al., J Immunol. 1995, 155 (3): 1165-74; Idusogie E E. et al., J Immunol. 2001, 166 (4): 2571-5; Lazar G A. et al., PNAS, 2006, 103 (11): 4005-4010; Ryan M C. et al., Mol. Cancer Ther., 2007, 6:3009-3018; Richards J O., et al., Mol Cancer Ther. 2008, 7 (8): 2517-27; Shields R. L. et al, J. Biol. Chem, 2002, 277:26733-26740; Shinkawa T. et al, J. Biol. Chem, 2003, 278:3466-3473.

In certain embodiments, the anti-CD276 antibodies or antigen-binding fragments comprise one or more amino acid substitution(s) that alters Complement Dependent Cytotoxicity (CDC), for example, by improving or diminishing C1q binding and/or CDC (see, for example, WO99/51642; Duncan & Winter Nature 322:738-40 (1988); U.S. Pat. Nos. 5,648,260; 5,624,821); and WO94/29351 concerning other examples of Fc region variants.

In certain embodiments, the anti-CD276 antibodies or antigen-binding fragments thereof comprise one or more amino acid substitution(s) in the interface of the Fc region to facilitate and/or promote heterodimerization. These modifications comprise introduction of a protuberance into a first Fc polypeptide and a cavity into a second Fc polypeptide, wherein the protuberance can be positioned in the cavity so as to promote interaction of the first and second Fc polypeptides to form a heterodimer or a complex. Methods of generating antibodies with these modifications are known in the art, e.g., as described in U.S. Pat. No. 5,731,168.

### Antigen-Binding Fragments

Provided herein are also anti-CD276 antigen-binding fragments. Various types of antigen-binding fragments are known in the art and can be developed based on the anti-CD276 antibodies provided herein, including for example, the exemplary antibodies whose CDR and FR sequences are shown in Table 1, and their different variants (such as affinity variants, glycosylation variants, Fc variants, cysteine-engineered variants and so on).

In certain embodiments, an anti-CD276 antigen-binding fragment provided herein is a camelized single domain antibody, a single chain Fv fragment (scFv), a dsFv, a (dsFv)₂, an Fv fragment, a Fab, a Fab′, a F(ab′)₂, a nanobody, a domain antibody, a single domain antibody, or a bivalent domain antibody.

Various techniques can be used for the production of such antigen-binding fragments. Illustrative methods include, enzymatic digestion of intact antibodies (see, e.g., Morimoto et al., Journal of Biochemical and Biophysical Methods 24:107-117 (1992); and Brennan et al., Science, 229:81 (1985)), recombinant expression by host cells such as E. Coli (e.g. for Fab, Fv and ScFv antibody fragments), screening from a phage display library as discussed above (e.g. for ScFv), and chemical coupling of two Fab′-SH fragments to form F(ab′)₂ fragments (Carter et al., Bio/Technology 10:163-167 (1992)). Other techniques for the production of antibody fragments will be apparent to a skilled practitioner.

In certain embodiments, the antigen-binding fragment is a scFv. Generation of scFv is described in, for example, WO 93/16185; U.S. Pat. Nos. 5,571,894; and 5,587,458. scFv may be fused to an effector protein at either the amino or the carboxy terminus to provide for a fusion protein (see, for example, Antibody Engineering, ed. Borrebaeck).

### Conjugates

In some embodiments, the anti-CD276 antibodies and antigen-binding fragments thereof is linked to one or more conjugates, optionally, wherein the conjugate is covalently attached either directly or via a linker. A conjugate is a non-proteinaceous moiety that can be attached to the antibody or antigen-binding fragment thereof. It is contemplated that a variety of conjugates may be linked to the antibodies or antigen-binding fragments provided herein (see, for example, “Conjugate Vaccines”, Contributions to Microbiology and Immunology, J. M. Cruse and R. E. Lewis, Jr. (eds.), Carger Press, New York, (1989)). These conjugates may be linked to the antibodies or antigen-binding fragments by covalent binding, affinity binding, intercalation, coordinate binding, complexation, association, blending, or addition, among other methods. In some embodiments, the conjugate comprises a clearance-modifying agent, a chemotherapeutic agent, a toxin, a radioactive isotope, a lanthanide, a luminescent label, a fluorescent label, an enzyme-substrate label, a DNA-alkylators, a topoisomerase inhibitor, a tubulin-binders, or other anticancer drugs.

In certain embodiments, the antibodies and antigen-binding fragments disclosed herein may be engineered to contain specific sites outside the epitope binding portion that may be utilized for binding to one or more conjugates. For example, such a site may include one or more reactive amino acid residues, such as for example cysteine or histidine residues, to facilitate covalent linkage to a conjugate.

In certain embodiments, the antibodies may be linked to a conjugate indirectly, or through another conjugate. For example, the antibody or antigen-binding fragments may be conjugated to biotin, then indirectly conjugated to a second conjugate that is conjugated to avidin. The conjugate can be a toxin (e.g., a chemotherapeutic agent), a detectable label (e.g., a radioactive isotope, a lanthanide, a luminescent label, a fluorescent label, or an enzyme-substrate label).

A “toxin” can be any agent that is detrimental to cells or that can damage or kill cells. Examples of toxin include, without limitation, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin and analogs thereof, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Examples of detectable label may include a fluorescent labels (e.g. fluorescein, rhodamine, dansyl, phycoerythrin, or Texas Red), enzyme-substrate labels (e.g. horseradish peroxidase, alkaline phosphatase, luceriferases, glucoamylase, lysozyme, saccharide oxidases or β-D-galactosidase), radioisotopes (e.g. ¹²³I, ¹²⁴I, ¹²⁵I, ¹³¹I, ³⁵S, ³H, ¹¹¹In, ¹¹²In, ¹⁴C, ⁶⁴Cu, ⁶⁷Cu, ⁸⁶Y, ⁸⁸Y, ⁹⁰Y, ¹⁷⁷Lu, ²¹¹At, ¹⁸⁶Re, ¹⁸⁸Re, ¹⁵³Sm, ²¹²Bi, and ³²P, other lanthanides, luminescent labels), chromophoric moiety, digoxigenin, biotin/avidin, a DNA molecule or gold for detection.

In certain embodiments, the conjugate can be a pharmacokinetic modifying moiety which helps increase half-life of the antibody. Illustrative example include water-soluble polymers, such as PEG, carboxymethylcellulose, dextran, polyvinyl alcohol, polyvinyl pyrrolidone, copolymers of ethylene glycol/propylene glycol, and the like. The polymer may be of any molecular weight, and may be branched or unbranched. The number of polymers attached to the antibody may vary, and if more than one polymer are attached, they can be the same or different molecules.

In certain embodiments, the conjugate can be a purification moiety such as a magnetic bead.

In certain embodiments, the antibodies and/or antigen-binding fragments thereof provided herein is used for a base for a conjugate.

### Polynucleotides and Recombinant Methods

The present disclosure provides isolated polynucleotides that encode the anti-CD276 antibodies and antigen-binding fragments thereof. DNA encoding the monoclonal antibody is readily isolated and sequenced using conventional procedures (e.g., by using oligonucleotide probes that are capable of binding specifically to genes encoding the heavy and light chains of the antibody). The encoding DNA may also be obtained by synthetic methods.

The isolated polynucleotide that encodes the anti-CD276 antibodies and antigen-binding fragments thereof can be inserted into a vector for further cloning (amplification of the DNA) or for expression, using recombinant techniques known in the art. Many vectors are available. The vector components generally include, but are not limited to, one or more of the following: a signal sequence, an origin of replication, one or more marker genes, an enhancer element, a promoter (e.g. SV40, CMV, EF-1α), and a transcription termination sequence.

In some embodiments, the vector system includes mammalian, bacterial, yeast systems, etc, and comprises plasmids such as, but not limited to, pALTER, pBAD, pcDNA, pCal, pL, pET, pGEMEX, pGEX, pCI, pCMV, PEGFP, pEGFT, pSV2, pFUSE, pVITRO, pVIVO, pMAL, pMD18-T, pMONO, pSELECT, pUNO, pDUO, Psg5L, pBABE, pWPXL, pBI, p15TV-L, pPro18, pTD, pRS420, pLexA, pACT2.2 etc, and other laboratorial and commercially available vectors. Suitable vectors may include, plasmid, or viral vectors (e.g., replication defective retroviruses, adenoviruses and adeno-associated viruses).

Vectors comprising the polynucleotide sequence encoding the antibody or antigen-binding fragment can be introduced to a host cell for cloning or gene expression. Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

In addition to prokaryotes, eukaryotic microbes such as filamentous fungi or yeast are suitable cloning or expression hosts for anti-CD276 antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated antibodies or antigen-fragment provided here are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruiffly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for anti-CD276 antibody production and cultured in conventional nutrient media modified as appropriate for inducing promoters, selecting transformants, or amplifying the genes encoding the desired sequences. In another embodiment, the antibody may be produced by homologous recombination known in the art.

The host cells used to produce the antibodies or antigen-binding fragments provided herein may be cultured in a variety of media. Commercially available media such as Ham's F10 (Sigma), Minimal Essential Medium (MEM), (Sigma), RPMI-1640 (Sigma), and Dulbecco's Modified Eagle's Medium (DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The anti-CD276 antibodies and antigen-binding fragments thereof prepared from the cells can be purified using, for example, hydroxylapatite chromatography, gel electrophoresis, dialysis, DEAE-cellulose ion exchange chromatography, ammonium sulfate precipitation, salting out, and affinity chromatography, with affinity chromatography being the preferred purification technique.

In certain embodiments, Protein A immobilized on a solid phase is used for immunoaffinity purification of the antibody and antigen-binding fragment thereof. The suitability of protein A as an affinity ligand depends on the species and isotype of any immunoglobulin Fc domain that is present in the antibody. Protein A can be used to purify antibodies that are based on human gamma1, gamma2, or gamma4 heavy chains (Lindmark et al., J. Immunol. Meth. 62:1-13 (1983)). Protein G is recommended for all mouse isotypes and for human gamma3 (Guss et al., EMBO J. 5:1567 1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

### CAR

In another aspect, the present disclosure also provides chimeric antigen receptor (CAR) comprising an antigen binding domain, a transmembrane domain, a costimulatory signaling region, and a TCR signaling domain, wherein the antigen binding domain specifically binds to CD276 and comprises an antigen binding fragment provided herein. In certain embodiments, the antigen binding fragment is a Fab or a scFv. In certain embodiments, the CAR provided herein is bispecific. The CAR is capable of further specifically binding to a second antigen other than CD276, or a second epitope on CD276. In certain embodiments, the second antigen is a tumor antigen, tumor associated antigen, or an immune related target as mentioned above. The TCR signaling domain can be selected from the group consisting of: an intracellular signal regions sequence of CD3ζ, FccRIγ, CD27, CD28, CD137, CD134, MyD88, CD40, CD278, TLRs, or a combination thereof. The transmembrane region can comprise a transmembrane region of CD3, CD4, CD8 or CD28. In certain embodiments, the second antigen is an immunoinhibitory molecule, such as PD-L1, SIRPα, CD47 or B2M.

In another aspect, the present disclosure also provides a nucleic acid sequence encoding the CAR as mentioned above as well as a cell or a vector comprising such nucleic acid sequence or a cell genetically modified to express the CAR provided herein. The cell can be an immune cell, optionally, wherein the immune cell is T lymphocyte, NK cell, monocyte, macrophage or NKT lymphocyte.

### Pharmaceutical Composition

The present disclosure further provides pharmaceutical compositions comprising the anti-CD276 antibodies or antigen-binding fragments thereof and one or more pharmaceutically acceptable carriers.

Pharmaceutical acceptable carriers for use in the pharmaceutical compositions disclosed herein may include, for example, pharmaceutically acceptable liquid, gel, or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

Suitable components may include, for example, antioxidants, fillers, binders, disintegrants, buffers, preservatives, lubricants, flavorings, thickeners, coloring agents, emulsifiers or stabilizers such as sugars and cyclodextrins. Suitable antioxidants may include, for example, methionine, ascorbic acid, EDTA, sodium thiosulfate, platinum, catalase, citric acid, cysteine, thioglycerol, thioglycolic acid, thiosorbitol, butylated hydroxanisol, butylated hydroxytoluene, and/or propyl gallate. As disclosed herein, inclusion of one or more antioxidants such as methionine in a composition comprising an antibody or antigen-binding fragment and conjugates as provided herein decreases oxidation of the antibody or antigen-binding fragment. This reduction in oxidation prevents or reduces loss of binding affinity, thereby improving antibody stability and maximizing shelf-life. Therefore, in certain embodiments compositions are provided that comprise one or more antibodies or antigen-binding fragments thereof as disclosed herein and one or more antioxidants such as methionine. Further provided are methods for preventing oxidation of, extending the shelf-life of, and/or improving the efficacy of an antibody or antigen-binding fragment as provided herein by mixing the antibody or antigen-binding fragment with one or more antioxidants such as methionine.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

The pharmaceutical compositions can be a liquid solution, suspension, emulsion, pill, capsule, tablet, sustained release formulation, or powder. Oral formulations can include standard carriers such as pharmaceutical grades of mannitol, lactose, starch, magnesium stearate, polyvinyl pyrollidone, sodium saccharine, cellulose, magnesium carbonate, etc.

In certain embodiments, the pharmaceutical compositions are formulated into an injectable composition. The injectable pharmaceutical compositions may be prepared in any conventional form, such as for example liquid solution, suspension, emulsion, or solid forms suitable for generating liquid solution, suspension, or emulsion. Preparations for injection may include sterile and/or non-pyretic solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile and/or non-pyretic emulsions. The solutions may be either aqueous or nonaqueous.

In certain embodiments, unit-dose parenteral preparations are packaged in an ampoule, a vial or a syringe with a needle. All preparations for parenteral administration should be sterile and not pyretic, as is known and practiced in the art.

In certain embodiments, a sterile, lyophilized powder is prepared by dissolving an antibody or antigen-binding fragment as disclosed herein in a suitable solvent. The solvent may contain an excipient which improves the stability or other pharmacological components of the powder or reconstituted solution, prepared from the powder. Excipients that may be used include, but are not limited to, water, dextrose, sorbital, fructose, corn syrup, xylitol, glycerin, glucose, sucrose or other suitable agents. The solvent may contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides a desirable formulation. In one embodiment, the resulting solution will be apportioned into vials for lyophilization. Each vial can contain a single dosage or multiple dosages of the anti-CD276 antibody or antigen-binding fragment thereof or composition thereof. Overfilling vials with a small amount above that needed for a dose or set of doses (e.g., about 10%) is acceptable so as to facilitate accurate sample withdrawal and accurate dosing. The lyophilized powder can be stored under appropriate conditions, such as at about 4° C. to room temperature.

Reconstitution of a lyophilized powder with water for injection provides a formulation for use in parenteral administration. In one embodiment, for reconstitution the sterile and/or non-pyretic water or other liquid suitable carrier is added to lyophilized powder. The precise amount depends upon the selected therapy being given, and can be empirically determined.

### Methods of Use

The present disclosure also provides therapeutic methods comprising: administering a therapeutically effective amount of the antibody or antigen-binding fragment thereof as provided herein to a subject in need thereof, thereby treating or preventing a CD276-related disease or condition. In some embodiment, the CD276-related disease or condition is cancer, autoimmune disease, inflammatory disease, adaptive immune disease or infectious disease.

Examples of cancer include but are not limited to, non-small cell lung cancer (squamous/nonsquamous), small cell lung cancer, renal cell cancer, colorectal cancer, colon cancer, ovarian cancer, breast cancer (including basal breast carcinoma, ductal carcinoma and lobular breast carcinoma), pancreatic cancer, gastric carcinoma, bladder cancer, esophageal cancer, mesothelioma, melanoma, head and neck cancer, thyroid cancer, sarcoma, prostate cancer, glioblastoma, cervical cancer, thymic carcinoma, melanoma, myelomas, mycoses fungoids, merkel cell cancer, hepatocellular carcinoma (HCC), fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, lymphoid malignancy, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, hepatoma, bile duct carcinoma, choriocarcinoma, Wilms' tumor, cervical cancer, testicular tumor, seminoma, classical Hodgkin lymphoma (CHL), primary mediastinal large B-cell lymphoma, T-cell/histiocyte-rich B-cell lymphoma, acute lymphocytic leukemia, acute myelocytic leukemia, acute myelogenous leukemia, chronic myelocytic (granulocytic) leukemia, chronic myelogenous leukemia, chronic lymphocytic leukemia, polycythemia vera, mast cell derived tumors, EBV-positive and -negative PTLD, and diffuse large B-cell lymphoma (DLBCL), plasmablastic lymphoma, extranodal NK/T-cell lymphoma, nasopharyngeal carcinoma, HHV8-associated primary effusion lymphoma, non-Hodgkin's lymphoma, multiple myeloma, Waldenstrom's macroglobulinemia, heavy chain disease, myelodysplastic syndrome, hairy cell leukemia and myelodysplasia, primary CNS lymphoma, spinal axis tumor, brain stem glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma.

In certain embodiments, the cancer is adrenal gland tumor, an AIDS-associated cancer, an alveolar soft part sarcoma, an astrocytic tumor, bladder cancer, bone cancer, a brain and spinal cord cancer, a metastatic brain tumor, a breast cancer, a carotid body tumors, a cervical cancer, a chondrosarcoma, a chordoma, a chromophobe renal cell carcinoma, a clear cell carcinoma, a colon cancer, a colorectal cancer, a cutaneous benign fibrous histiocytoma, a desmoplastic small round cell tumor, an ependymoma, a Ewing's tumor, an extraskeletal myxoid chondrosarcoma, a fibrogenesis imperfecta ossium, a fibrous dysplasia of the bone, a gallbladder or bile duct cancer, gastric cancer, a gestational trophoblastic disease, a germ cell tumor, a head and neck cancer, hepatocellular carcinoma, an islet cell tumor, a Kaposi's Sarcoma, a kidney cancer, a leukemia, a lipoma/benign lipomatous tumor, a liposarcoma/malignant lipomatous tumor, a liver cancer, a lymphoma, a lung cancer, a medulloblastoma, a melanoma, a meningioma, a multiple endocrine neoplasia, a multiple myeloma, a myelodysplastic syndrome, a neuroblastoma, a neuroendocrine tumors, an ovarian cancer, a pancreatic cancer, a papillary thyroid carcinoma, a parathyroid tumor, a pediatric cancer, a peripheral nerve sheath tumor, a phaeochromocytoma, a pituitary tumor, a prostate cancer, a posterior uveal melanoma, a rare hematologic disorder, a renal metastatic cancer, a rhabdoid tumor, a rhabdomysarcoma, a sarcoma, a skin cancer, a soft-tissue sarcoma, a squamous cell cancer, a stomach cancer, a synovial sarcoma, a testicular cancer, a thymic carcinoma, a thymoma, a thyroid metastatic cancer, and a uterine cancer. In certain embodiments, the cancer is chemoresistant.

In certain embodiments, the disease or condition is hematological cancer chosen from B-cell lymphomas. Examples of B-cell lymphomas includes but not limited to, Hodgkin's lymphoma, non-Hodgkin's lymphoma (NHL), acute lymphocytic leukemia (ALL), acute myeloid leukemia (AML), chronic lymphocytic leukemia (CLL), chronic myelogenous leukemia (CML), multiple myeloma (MM), diffuse large B cell lymphoma (DLBCL), Marginal zone B-cell lymphoma (MZL), Mantle cell lymphoma (MCL), Richter's syndrome, Burkitt's lymphoma or follicular lymphoma.

Autoimmune diseases include, but are not limited to, Acquired Immunodeficiency Syndrome (AIDS, which is a viral disease with an autoimmune component), alopecia areata, ankylosing spondylitis, antiphospholipid syndrome, autoimmune Addison's disease, autoimmune hemolytic anemia, autoimmune hepatitis, autoimmune inner ear disease (AIED), autoimmune lymphoproliferative syndrome (ALPS), autoimmune thrombocytopenia purpura (ATP), Behcet's disease, cardiomyopathy, celiac sprue-dermatitis hepetiformis; chronic fatigue immune dysfunction syndrome (CFIDS), chronic inflammatory demyelinating polyneuropathy (CIPD), cicatricial pemphigold, cold agglutinin disease, crest syndrome, Crohn's disease, Degos' disease, dermatomyositis-juvenile, discoid lupus, essential mixed cryoglobulinemia, fibromyalgia-fibromyositis, Graves' disease, Guillain-Barre syndrome, Hashimoto's thyroiditis, idiopathic pulmonary fibrosis, idiopathic thrombocytopenia purpura (ITP), IgA nephropathy, insulin-dependent diabetes mellitus, juvenile chronic arthritis (Still's disease), juvenile rheumatoid arthritis, Meniere's disease, mixed connective tissue disease, multiple sclerosis, myasthenia gravis, pemacious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomena, Reiter's syndrome, rheumatic fever, rheumatoid arthritis, sarcoidosis, scleroderma (progressive systemic sclerosis (PSS), also known as systemic sclerosis (SS)), Sjogren's syndrome, stiff-man syndrome, systemic lupus erythematosus, Takayasu arteritis, temporal arteritis/giant cell arteritis, ulcerative colitis, uveitis, vitiligo and Wegener's granulomatosis.

Inflammatory disorders, include, for example, chronic and acute inflammatory disorders. Examples of inflammatory disorders include Alzheimer's disease, asthma, atopic allergy, allergy, atherosclerosis, bronchial asthma, eczema, glomerulonephritis, graft vs. host disease, hemolytic anemias, osteoarthritis, sepsis, stroke, transplantation of tissue and organs, vasculitis, diabetic retinopathy and ventilator induced lung injury. In some embodiments, the CD276 associated conditions are inflammatory diseases such as systemic lupus erythematosus (SLE), intestinal mucosal inflammation, wasting disease associated with colitis, multiple sclerosis, viral infections, rheumatoid arthritis, osteoarthritis, Cohn's disease, and inflammatory bowel disease, psoriasis, systemic scleroderma, autoimmune diabetes and the like.

Infectious disease include, but are not limited to, fungus infection, parasite/protozoan infection or chronic viral infection, for example, malaria, coccidioiodmycosis immitis, histoplasmosis, onychomycosis, aspergilosis, blastomycosis, candidiasis albicans, paracoccidioiomycosis, microsporidiosis, Acanthamoeba keratitis, Amoebiasis, Ascariasis, Babesiosis, Balantidiasis, Baylisascariasis, Chagas disease, Clonorchiasis, Cochliomyia, Cryptosporidiosis, Diphyllobothriasis, Dracunculiasis, Echinococcosis, Elephantiasis, Enterobiasis, Fascioliasis, Fasciolopsiasis, Filariasis, Giardiasis, Gnathostomiasis, Hymenolepiasis, Isosporiasis, Katayama fever, Leishmaniasis, Lyme disease, Metagonimiasis, Myiasis, Onchocerciasis, Pediculosis, Scabies, Schistosomiasis, Sleeping sickness, Strongyloidiasis, Taeniasis, Toxocariasis, Toxoplasmosis, Trichinosis, Trichuriasis, Trypanosomiasis, helminth infection, infection of hepatitis B (HBV), hepatitis C (HCV), herpes virus, Epstein-Barr virus, HIV, cytomegalovirus, herpes simplex virus type I, herpes simplex virus type II, human papilloma virus, adenovirus, human immunodeficiency virus I, human immunodeficiency virus II, Kaposi West sarcoma associated herpes virus epidemics, thin ring virus (Torquetenovirus), human T lymphotrophic viruse I, human T lymphotrophic viruse II, varicella zoster, JC virus or BK virus.

In certain embodiments, the subject is human.

In another aspect, methods are provided to treat a disease or condition in a subject that would benefit from modulation of CD276 activity, comprising administering a therapeutically effective amount of the antibody or antigen-binding fragment thereof as provided herein to a subject in need thereof. The term “disease or condition” as used herein can be used interchangeably with the term “CD276-related disease or condition”.

The therapeutically effective amount of an antibody or antigen-binding fragment thereof as provided herein will depend on various factors known in the art, such as for example body weight, age, past medical history, present medications, state of health of the subject and potential for cross-reaction, allergies, sensitivities and adverse side-effects, as well as the administration route and extent of disease development. Dosages may be proportionally reduced or increased by one of ordinary skill in the art (e.g., physician or veterinarian) as indicated by these and other circumstances or requirements.

In certain embodiments, an antibody or antigen-binding fragment thereof as provided herein may be administered at a therapeutically effective dosage of about 0.01 mg/kg to about 100 mg/kg (e.g., about 0.01 mg/kg, about 0.5 mg/kg, about 1 mg/kg, about 2 mg/kg, about 3 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg). In certain of these embodiments, the antibody or antigen-binding fragment thereof is administered at a dosage of about 50 mg/kg or less, and in certain of these embodiments the dosage is 10 mg/kg or less, 5 mg/kg or less, 3 mg/kg or less, 1 mg/kg or less, 0.5 mg/kg or less, or 0.1 mg/kg or less. In certain embodiments, the administration dosage may change over the course of treatment. For example, in certain embodiments, the initial administration dosage may be higher than subsequent administration dosages. In certain embodiments, the administration dosage may vary over the course of treatment depending on the reaction of the subject.

Dosage regimens may be adjusted to provide the optimum desired response (e.g., a therapeutic response). For example, a single dose may be administered, or several divided doses may be administered over time.

The antibodies and antigen-binding fragments thereof disclosed herein may be administered by any route known in the art, such as for example parenteral (e.g., subcutaneous, intraperitoneal, intravenous, including intravenous infusion, intramuscular, or intradermal injection) or non-parenteral (e.g., oral, intranasal, intraocular, sublingual, rectal, or topical) routes.

In some embodiments, the antibodies or antigen-binding fragments thereof disclosed herein may be administered alone or in combination with one or more additional therapeutic means or agents. In some embodiments, the antibodies or antigen-binding fragments thereof disclosed herein may be administered alone or in combination with a second therapeutic agent. For example, the antibodies or antigen-binding fragments thereof disclosed herein may be administered in combination with a second therapeutic agent, for example, a chemotherapeutic agent or an anti-cancer drug. In certain embodiments, the antibodies or antigen-binding fragments thereof disclosed herein may be administered in combination with an antagonist of one or more immunoinhibitory molecule, e.g., CD24, CD47, SIRPα, PD-L1, or the beta-2 microglobulin subunit of the major histocompatibility class I complex (B2M). The term “antagonist” as used herein comprises can refer to any small molecule, small or micro RNAs, or antibodies or antigen-binding fragments thereof that blocks or inhibits binding of CD24, CD47, SIRPα, PD-L1 or B2M to their respective binding partners so as to prevent elicit of immunoinhibitory signals. In certain embodiments, the antibodies or antigen-binding fragments thereof disclosed herein is administered in combination with a PD-L1 antagonist, such as an anti-PD-L1 antibody or antigen-binding fragment thereof.

The term “anti-PD-L1 antibody” can refer to any known anti-PD-L1 antibodies or antigen-binding fragment thereof, including without limitation, YN035 as disclosed in WO2019196309A1, and MPDL3280A as disclosed in WO2010077634A1.

In certain of these embodiments, an antibody or antigen-binding fragment thereof as disclosed herein that is administered in combination with one or more additional therapeutic agents may be administered simultaneously with the one or more additional therapeutic agents, and in certain of these embodiments the antibody or antigen-binding fragment thereof and the additional therapeutic agent(s) may be administered as part of the same pharmaceutical composition. However, an antibody or antigen-binding fragment thereof administered “in combination” with another therapeutic agent does not have to be administered simultaneously with or in the same composition as the agent. An antibody or antigen-binding fragment thereof administered prior to or after another agent is considered to be administered “in combination” with that agent as the phrase is used herein, even if the antibody or antigen-binding fragment thereof and a second agent are administered via different routes. Where possible, additional therapeutic agents administered in combination with the antibodies or antigen-binding fragments thereof disclosed herein are administered according to the schedule listed in the product information sheet of the additional therapeutic agent, or according to the Physicians' Desk Reference 2003 (Physicians' Desk Reference, 57th Ed; Medical Economics Company; ISBN: 1563634457; 57th edition (November 2002)) or protocols well known in the art.

In some embodiments, the present disclosure provides methods of detecting presence or amount of CD276 in a sample, comprising contacting the sample with the antibody or antigen-binding fragment thereof, and determining the presence or the amount of CD276 in the sample.

In some embodiments, the present disclosure provides methods of diagnosing a CD276 related disease or condition in a subject, comprising: a) obtaining a sample from the subject; b) contacting the sample obtained from the subject with the antibody or antigen-binding fragment thereof provided herein; c) determining presence or amount of CD276 in the sample; and d) correlating the existence of the CD276 to the CD276 related disease or condition in the subject.

In some embodiments, the present disclosure also provides use of the antibody or antigen-binding fragment thereof provided herein in the manufacture of a medicament for treating a CD276 related disease or condition in a subject, in the manufacture of a diagnostic reagent for diagnosing a CD276 related disease or condition.

In another aspect, the present disclosure also provides a method of modulating CD276 activity in a CD276-expressing cell, comprising exposing the CD276-expressing cell to the antibody or antigen-binding fragment thereof provided herein.

In another aspect, the present disclosure also provides a method of detecting presence or amount of CD276 in a sample, comprising contacting the sample with the antibody or antigen-binding fragment thereof provided herein, and determining the presence or the amount of CD276 in the sample.

In another aspect, the present disclosure also provides a method of diagnosing a CD276 related disease or condition in a subject, comprising: a) obtaining a sample from the subject; b) contacting the sample obtained from the subject with the antibody or antigen-binding fragment thereof provided herein; c) determining presence or amount of CD276 in the sample; and d) correlating the presence or the amount of CD276 to existence or status of the CD276 related disease or condition in the subject.

In another aspect, the present disclosure also provides use of the antibody or antigen-binding fragment thereof provided herein in the manufacture of a medicament for treating a CD276 related disease or condition in a subject. The medicament can further comprise a second therapeutic agent, e.g., a PD-L1 antagonist, optionally, wherein the PD-L1 antagonist is an anti-PD-L1 antibody or antigen-binding fragment thereof.

In another aspect, the present disclosure also provides use of the antibody or antigen-binding fragment thereof provided herein in the manufacture of a diagnostic reagent for diagnosing a CD276 related disease or condition.

In another aspect, the present disclosure also provides a kit comprising the antibody or antigen-binding fragment thereof provided herein, useful in detecting CD276, optionally recombinant CD276, CD276 expressed on cell surface, or CD276-expressing cells. The term “recombinant” as used herein refers to artificial manipulation of one or more biological molecules such as polynucleotide or polypeptide molecules using one or more molecular biology techniques to make such biological molecule(s) into something other than its natural state.

In another aspect, the present disclosure also provides a method for stimulating a T cell-mediated immune response to a CD276-expressing cell or tissue in a mammal, the method comprising administering to the mammal an effective amount of a cell genetically modified to express the CAR comprising an antigen binding domain, a transmembrane domain, a costimulatory signaling region, and a TCR signaling domain, wherein the antigen binding domain specifically binds to CD276 and comprises an antigen binding fragment provided herein. In certain embodiments, the antigen binding fragment is a Fab or a scFv. In certain embodiments, the CAR provided herein is bispecific. The CAR is capable of further specifically binding to a second antigen other than CD276 or a second epitope on CD276. In certain embodiments, the second antigen is a tumor antigen as mentioned above. The TCR signaling domain can be selected from the group consisting of: an intracellular signal region sequence of CD3ζ, FccRIγ, CD27, CD28, CD137, CD134, MyD88, CD40, CD278, TLRs, or a combination thereof. The transmembrane region can comprise a transmembrane region of CD3, CD4, CD8 or CD28. In certain embodiments, the second antigen is an immunoinhibitory molecule, e.g., PD-L1, SIRPα, CD24, CD47 or B2M.

In another aspect, the present disclosure also provides method for treating a mammal having a CD276 related disease or condition, comprising administering to the mammal an effective amount of a cell that is genetically modified to express the CAR provided herein (e.g., an autologous T cell), thereby treating the mammal. In certain embodiments, the CD276 related disease or condition is cancer. In certain embodiments, the mammal is a human subject.

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. One skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

### Example 1: Hybridoma Development

### 1. Methods

### 1.1 Immunization and Serum Titer Determination

### 1.1.1 Immunogen and Immunization Strategies

### Cell Immunization

CHO-S cells overexpressing human CD276 protein (UniProt ID: Q5ZPR3, i.e. CHO-S-hCD276) or mouse CD276 protein (UniProt ID: Q8VE98, i.e. CHO-S-hCD276) were used as immunogen.

### Protein Immunization

Recombinant human CD276 protein (SEQ ID NO: 346): the recombinant human CD276 protein was prepared by digesting the human CD276 protein with enterokinase, and the extracellular domain of human CD276 was fused with 6×His tag and DDDDK (SEQ ID NO: 345).

Recombinant human CD276 protein (SEQ ID NO: 346):

LEVQVPEDPVVALVGTDATLCCSFSPEPGFSLAQLNLIWQLTDTKQLVHS
FAEGDQGSAYANRTALFPDLLAQGNASLRLQRVRVADEGSFTCFVSIRDF
GSAAVSLQVAAPYSKPSMTLEPNKDLRPGDTVTITCSSYQGYPEAEVFWQ
DGQGVPLTGNVTTSQMANEQGLFDVHSILRVVLGANGTYSCLVRNPVLQQ
DAHSSVTITPQRSPTGAVEVQVPEDPVVALVGTDATLRCSFSPEPGFSLA
QLNLIWQLTDTKQLVHSFTEGRDQGSAYANRTALFPDLLAQGNASLRLQR
VRVADEGSFTCFVSIRDFGSAAVSLQVAAPYSKPSMTLEPNKDLRPGDTV
TITCSSYRGYPEAEVFWQDGQGVPLTGNVTTSQMANEQGLFDVHSVLRVV
LGANGTYSCLVRNPVLQQDAHGSVTITGQPMTFPPEAHHHHHHDDDDK.

Balb/c and SJL mice were immunized as shown below. The primary immunization was followed by several boosts until animals developed satisfactory antiserum titers suitable for hybridoma development.

| | | | Animal/ | Group | |
|---|---|---|---|---|---|
| Group | Immunogen | Route | Strain | size | Dosage |
| 1 | CHO-S-hCD276 cells | Intraperitoneal | Balb/c | 5 | 5 × 10⁶ |
| | | Injection (I.P.) | | | |
| 2 | Recombinant human | hypodermic | Balb/c | 5 | 100 μg |
| | CD276 protein | injection (I.H.) | | | |
| 3 | CHO-S-mCD276 | I.P. | Balb/c | 5 | 5 × 10⁶ |
| 4 | CHO-S-hCD276 cells | I.P. | SJL | 5 | 5 × 10⁶ |
| 5 | Recombinant human | I.H. | SJL | 5 | 100 μg |
| | CD276 protein | | | | |

### 1.1.2 Immunization Schedules

### Immunization Schedule (Group 1)

| | |
|---|---|
| Day 0 | Pre-bleed (15-30 μL serum/mouse) |
| | Primary: 5 × 10⁶ cell per mouse I.P. |
| Day 14 | Boost 1: 5 × 10⁶ cell per mouse I.P. |
| | Test Bleed (15-30 μL serum/mouse) (TB1) |
| | Test bleed FACS |
| Day 28 | Boost 2: I.P., 5 × 10⁶ cell per mouse I.P. |
| Day 38 | Test Bleed (15-30 μL serum/mouse) (TB2) |
| Day 38 | Test bleed FACS |
| | Data analysis and phase conclusion |
| Day 39 | Pre-fusion (final) Boost, I.H., 100 μg per mouse |

Animals not selected for cell fusion will be maintained in cage and may be given additional boost immunizations.

### Immunization Schedule (Group 2)

| | |
|---|---|
| Day 0 | Pre-bleed (15-30 μL serum/mouse) |
| | Primary: 100 μg/mouse I.H., CFA |
| Day 14 | Boost 1: 100 μg/mouse I.H., IFA |
| | Test Bleed (15-30 μL serum/mouse) (TB1) |
| | Test bleed FACS |
| Day 28 | Boost 2: I.H., 100 μg/mouse I.H., IFA |
| Day 38 | Test Bleed (15-30 μL serum/mouse) (TB2) |
| Day 38 | Test bleed FACS |
| | Data analysis and phase conclusion |
| Day 42 | Pre-fusion (final) Boost, I.P., 5 × 10⁶ cell per mouse |

Animals not selected for cell fusion will be maintained in cage and may be given additional boost immunizations.

### Immunization Schedule (Group 3)

| | |
|---|---|
| Day 0 | Pre-bleed (15-30 μL serum/mouse) |
| | Primary: 5 × 10⁶ cell per mouse I.P. |
| Day 14 | Boost 1: 5 × 10⁶ cell per mouse I.P. |
| | Test Bleed (15-30 μL serum/mouse) (TB1) |
| | Test bleed FACS |
| Day 28 | Boost 2: I.P., 5 × 10⁶ cell per mouse I.P. |
| Day 38 | Test Bleed (15-30 μL serum/mouse) (TB2) |
| Day 38 | Test bleed FACS |
| | Data analysis and phase conclusion |
| Day 39 | Pre-fusion (final) Boost, I.P., 5 × 10⁶ cell per mouse |

Animals not selected for cell fusion were maintained in cage and were given additional boost immunizations.

### Immunization Schedule (Group 4)

| | |
|---|---|
| Day 0 | Pre-bleed (15-30 μL serum/mouse) |
| | Primary: 5 × 10⁶ cell per mouse I.P. |
| Day 14 | Boost 1: 5 × 10⁶ cell per mouse I.P. |
| | Test Bleed (15-30 μL serum/mouse) (TB1) |
| | Test bleed FACS |
| Day 28 | Boost 2: I.P., 5 × 10⁶ cell per mouse I.P. |
| Day 38 | Test Bleed (15-30 μL serum/mouse) (TB2) |
| Day 38 | Test bleed FACS |
| | Data analysis and phase conclusion |
| Day 42 | Boost 2: I.P., 5 × 10⁶ cell per mouse I.P. |
| Day 51 | Test Bleed (15-30 μL serum/mouse) (TB2) |
| Day 51 | Test bleed FACS |
| | Data analysis and phase conclusion |
| Day 52 | Pre-fusion (final) Boost, I.P., 5 × 10⁶ cell per mouse |

Animals not selected for cell fusion were maintained in cage and were given additional boost immunizations.

### Immunization Schedule (Group 5)

| | |
|---|---|
| Day 0 | Pre-bleed (15-30 μL serum/mouse) |
| | Primary: 100 μg/mouse I.H., CFA |
| Day 14 | Boost 1: 100 μg/mouse I.H., IFA |
| | Test Bleed (15-30 μL serum/mouse) (TB1) |
| | Test bleed FACS |
| Day 28 | Boost 2: I.H, 100 μg/mouse I.H., IFA |
| Day 38 | Test Bleed (15-30 μL serum/mouse) (TB2) |
| Day 38 | Test bleed FACS |
| Day 42 | Boost 2: I.H, 100 μg/mouse I.H., IFA |
| Day 51 | Test Bleed (15-30 μL serum/mouse) (TB2) |
| Day 51 | Test bleed FACS |
| | Data analysis and phase conclusion |
| Day 68 | Pre-fusion (final) Boost, I.H, 100 μg/mouse |

Animals not selected for cell fusion were maintained in cage and were given additional boost immunizations.

### 1.1.3 Test Bleed Antiserum Analysis

Screening-Test bleeds were performed and evaluated by testing using FACS on CHO-S cell line stably over-expressing human and/or mouse CD276 (CHO-S-hCD276 and/or CHO-S-mCD276).

Screening-Test bleeds were performed and evaluated by testing using Elisa with extra-cellular domain of recombinant human CD276 protein.

### 1.2 Hybridoma Generation and Screening

### 1.2.1 Cell Fusion and Screening

Fusion—Splenocyte fusions were performed on the mice which responded the best to the immunizations as determined by test bleed FACS. The lymphocytes from spleens and lymph nodes were fused to a Sp2/0 cell line using an optimized electrofusion protocol. Multiple fusions were performed to ensure success of the cell fusion.

Screening and expansion—The fusion was plated (2×10⁴ to 10⁵ per well) into a stack of 96-well plates. Plates were monitored for growth and fed weekly. Wells with cell growth were screened by primary screening assays in 10-14 days with FACS and/or other feasible assays such as Elisa. Multiple fusions for each targeting antigen were performed and screened. The positive parental clones which showed positive binding with CHO-S-CD276 and positive Elisa signal from primary screening were expanded into 24-well plates for secondary screening.

Additional antibody screening-Following primary screening, positive parental clones expanded into 24-well plates were screened again by the assay described in the hybridoma screening funnel below.

Hybridomas of interest were chosen to proceed to subcloning.

### 1.2.2 Hybridoma Subcloning, Screening and Cryopreservation

Subcloning—The parental hybridomas with desired reactivity and isotypes from the screening funnel above were then subcloned by multiple rounds of limiting dilution or single cell sorting until monoclones were obtained.

Screening & Expansion—The subcloning plates were screened by protein or cell-based Elisa, and the subclones with good binding ability were expanded to 24-wells for confirmation tests. The specificity and cross-reactivity of these subclones were confirmed with FACS analysis. Briefly, parental CHO-S cells, CHO-S-hCD276, CHO-S cell line stably over-expressing Macaca fascicularis CD276, CHO-K1 S, cell line stably over-expressing mouse CD276 were incubated with antibodies produced by each subclone respectively. Fluorescent dye-conjugated secondary antibody was used to detect the binding of the primary antibody with the cells. Median fluorescence intensity was measured by FACS analysis.

Cryopreservation—The desired subclonal cell lines were sequenced and further expanded into culture flasks for cryopreservation. 4-6 vials per cell line at 0.5-13.0×10⁶ cells/vial were initially cryopreserved. Master cell bank and working cell bank were established for the selected most valuable cell lines if desired.

### 2. Results

We discovered 43 antibodies with unique sequences showing positive binding with CHO-S cell stably over-expressing human CD276 protein (CHO-S-hCD276) but not binding to parental CHO-S cells, suggesting these antibodies are human CD276 recognizing antibody. Among which 42 antibodies could bind with cynomolgus monkey CD276, and 11 antibodies could bind with mouse CD276 protein. The MFI of the mouse antibodies staining CHO-S, CHO-S-hCD276, CHO-S-mCD276, CHO-S-Macaca fascicularis CD276 (CHO-S-cynoCD276), detected by FACS were summarized in the table below (Table 5).

TABLE 5
MFI of antibodies binding with different cell lines

| | FACS | FACS MFI | FACS MFI | FACS MFI |
|---|---|---|---|---|
| | MFI | (CHO-S- | CHO-S- | CHO-S- |
| Clone | (CHO-S) | hCD276) | mCD276 | cynoCD276 |
| 15-G1-D1-E3 | 1585 | 62545 | 6610 | 99442 |
| 28-G2-E6-B10 | 1267 | 56316 | 2077 | 40831 |
| 6-D8-E7-A11 | 2571 | 37901 | 92619 | 100797 |
| 8-B4-F5-E11 | 1574 | 40419 | 7366 | 58650 |
| 13-A8-C4-G1 | 999 | 54074 | 6198 | 95808 |
| 6-F3-G2-G1 | 1441 | 37392 | 6978 | 113858 |
| 15-C8-B5-G7 | 1114 | 36640 | 5734 | 42958 |
| 3-G7-D8-D3 | 1331 | 82411 | 1291 | 6422 |
| 14-E7-G9-D4 | 1331 | 78067 | 1304 | 61808 |
| 6-H11-G5-D8 | 1327 | 112516 | 1358 | 30365 |
| 7-E1-F8-F6 | 1345 | 6990 | 1309 | 5590 |
| 9-E8-F9-C10 | 1309 | 7607 | 1951 | 5705 |
| 3-C2-C3-E7 | 1188 | 38810 | 2770 | 2106 |
| 27-F8-E10-E11 | 1322 | 15962 | 1349 | 80481 |
| 9-G2-H6-E4 | 1309 | 49603 | 27620 | 5404 |
| 25-D3-G4-C6 | 1331 | 5941 | 1372 | 5386 |
| 27-E7-D8-C7 | 1313 | 94678 | 1587 | 87885 |
| 2-A7-B10-A3 | 1322 | 58948 | 2039 | 56030 |
| 18-F9-D8-G7 | 1309 | 126666 | 20645 | 83534 |
| 10-B9-D10-A12 | 1280 | 399656 | 3171 | 10125 |
| 24-C10-F9-G7 | 1342 | 85105 | 1184 | 15198 |
| 20-A2-D10-G8 | 1184 | 77149 | 1255 | 9025 |
| 3-F2-E7-F9 | 1250 | 52275 | 1141 | 35661 |
| 5-D1-G6-D9 | 1349 | 45271 | 1529 | 62228 |
| 23-F10-G4-F11 | 1327 | 127095 | 1300 | 36702 |
| 16-C6-F7-F5 | 1276 | 141395 | 1833 | 19524 |
| 22-E11-C3-F2 | 1188 | 74329 | 1667 | 56507 |
| 11-C12-F4-F6 | 1345 | 41880 | 1340 | 5137 |
| 16-G3-D10-C10 | 1336 | 23162 | 1322 | 6188 |
| 26-D2-D6-B12 | 1345 | 47467 | 1313 | 63076 |
| 21-B3-B1-H5 | 1304 | 71491 | 1345 | 20230 |
| 30-C7-C11-D4 | 1327 | 138317 | 8222 | 96294 |
| 11-G10-B4-B11 | 1307 | 32768 | 2798 | 26386 |
| 20-F8-B5-G2 | 1322 | 88781 | 67038 | 51137 |
| 9-B9-H11-G7 | 1041 | 96785 | 1356 | 145769 |
| 9-G12-D6-A11 | 1066 | 53167 | 1422 | 35661 |
| 10-G6-C4-B2 | 1340 | 5647 | 17431 | 4322 |
| 8-C3-E3-F3 | 1340 | 63936 | 1322 | 12053 |
| 30-E2-G7-G7 | 1322 | 46988 | 1296 | 35122 |
| 20-C5-D7-D3 | 1309 | 77017 | 1345 | 13206 |
| 13-E4-G9-A4 | 1209 | 148759 | 1370 | 16822 |
| 25-C8-D7-C5 | 1188 | 147755 | 1157 | 39074 |
| 4-D5-B9-B11 | 1225 | 144785 | 2099 | 148256 |

### Example 2: Antibody Characterization: Affinity

### 1. Methods

### 1.1 Cell Based Binding Affinity on SKOV3 Cancer Cell Line

Sequences of 31 mouse antibodies from Table 5 were selected to generate and produce human IgG1 chimeric antibodies. The binding affinity of these antibodies and bench mark antibody, Enoblituzumab (see U.S. Pat. No. 8,802,091, MGA271, specifically with a construct designated as hBRCA84D-2) and MGC018 (an antibody-drug-conjugate, in which the mAb MGA017 (human IgG1) is conjugated via a cleavable linker to the prodrug seco-DUocarmycin hydroxyBenzamide Azaindole (DUBA), an alkylating agent that can damage DNA in both dividing and non-dividing cells, thereby causing cell death) with human patient derived ovarian cancer cell line, SKOV3, was determined by FACS analysis.

The protocol for FACs analysis is described as follows:
1. Digested cells using Trypsin (1×); Centrifuged the harvested cells at 300 g for 3 min and discarding the supernatant.
2. Washed the cells 2 times with FACS buffer by centrifuging at 300 g for 3 min and discarding the supernatant.
3. Resuspended the cells, and 2*10⁵ cell/well was seeded into the assay plate in 50 μl FACS buffer, then added 50 μl primary antibody (primary antibody final concentration: (5.00, 1.67, 0.56, 0.19, 0.06, 0.02, 0.01, 0.00 μg/ml or 20.00, 6.67, 2.22, 0.74, 0.25, 0.08, 0.03, 0.00 μg/ml). Incubated at 4° C. for 1 hour.
4. Washed the cells twice by using the condition in step 2. Resuspended the cells with 100 μl/well diluted 2nd antibody, incubated at 4° C. for 1 hour in the dark.
5. Washed the cells twice by using the condition in step 2. Resuspended the cells with 100 μl/well FACS buffer. Kept the cells in dark for FACS analysis.

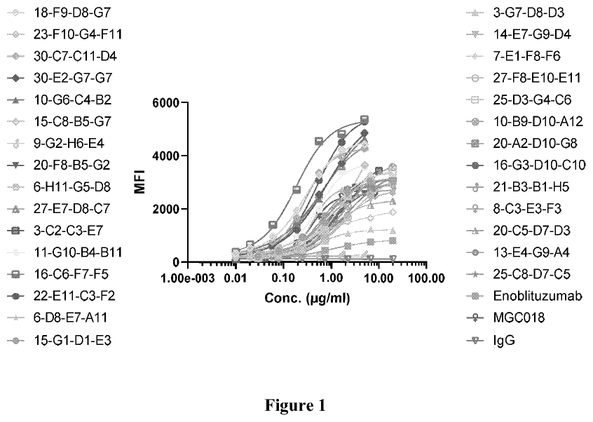

The binding affinity of the selected antibodies on SKOV3 are higher, lower or comparable with bench mark antibody Enoblituzumab (see Table 6 and FIG. 1).

TABLE 6
Binding affinity of the chimeric antibodies on SKOV3

SKOV3

| Number | Name | MFI (Max) | EC₅₀ (μg/ml) |
|---|---|---|---|
| 1 | igG | 114.8 | / |
| 2 | Enoblituzumab | 830.3 | 0.9847 |
| 3 | MGC018 | 2545 | 0.3847 |
| 4 | 15-G1-D1-E3 | 3186 | 0.8113 |
| 5 | 6-D8-E7-A11 | 2828 | 0.4022 |
| 6 | 15-C8-B5-G7 | 2137 | 0.2929 |
| 7 | 3-G7-D8-D3 | 1214 | 0.5095 |
| 8 | 14-E7-G9-D4 | 2726 | 1.618 |
| 9 | 6-H11-G5-D8 | 199.6 | 1.78 |
| 10 | 7-E1-F8-F6 | 3378 | 0.9823 |
| 11 | 3-C2-C3-E7 | 3703 | 1.433 |
| 12 | 27-F8-E10-E11 | 3224 | 2.26 |
| 13 | 9-G2-H6-E4 | 291.7 | 3.659 |
| 14 | 25-D3-G4-C6 | 3447 | 1.267 |
| 15 | 27-E7-D8-C7 | 4750 | 0.5425 |
| 16 | 18-F9-D8-G7 | 4610 | 0.2857 |
| 17 | 10-B9-D10-A12 | 3879 | 2.082 |
| 18 | 20-A2-D10-G8 | 3112 | 0.7906 |
| 19 | 23-F10-G4-F11 | 6725 | 4.425 |
| 20 | 16-C6-F7-F5 | 5354 | 0.1858 |
| 21 | 22-E11-C3-F2 | 5545 | 0.5228 |
| 22 | 16-G3-D10-C10 | 3176 | 1.311 |
| 23 | 21-B3-B1-H5 | 2718 | 0.7228 |
| 24 | 30-C7-C11-D4 | 4266 | 0.2821 |
| 25 | 11-G10-B4-B11 | 3747 | 0.5358 |
| 26 | 20-F8-B5-G2 | 3012 | 1.109 |
| 27 | 10-G6-C4-B2 | 5640 | 0.8215 |
| 28 | 8-C3-E3-F3 | 2883 | 0.6432 |
| 29 | 30-E2-G7-G7 | 5628 | 0.814 |
| 30 | 20-C5-D7-D3 | 2308 | 0.941 |
| 31 | 13-E4-G9-A4 | 3076 | 1.775 |
| 32 | 25-C8-D7-C5 | 3483 | 2.031 |

### 2. Protein Based Affinity Test by Biacore (ChemPartner)

### Test Condition:

Analyte: B7H3
Running buffer: HBS-EP⁺
Flow Rate: 30 μL/min
Capture: Abs, 10 μL/min for 60 s
Injection of serial diluted B7H3
Contact time: 180 s
Dissociation time: 400 s
Regeneration: pH1.5 Gly, 30 μL/min for 30 s
Method: Multiple cycle kinetics/affinity using capture
Machine Model: Biacore 8K (GE)
Analysis Temperature: 25° C.

TABLE 7
shows the binding affinity of the chimeric antibodies on hB7H3
by Biacore (ChemPartner)

| | Kinetics | Capture 1 | Analyte 1 | Kinetics | | | |
|---|---|---|---|---|---|---|---|
| Entry | model | Solution | Solution | Chi² (RU²) | ka (1/Ms) | kd (1/s) | KD (M) |
| 1 | 1:1 binding | 2 μg/ml 15- | HUMAN B7H3 | 5.91E−01 | 5.50E+05 | 2.11E−04 | 3.83E−10 |
| | | G1-D1-E3 | | | | | |
| 2 | 1:1 binding | 1 μg/ml 6- | HUMAN B7H3 | 8.17E−01 | 4.64E+05 | 3.48E−04 | 7.49E−10 |
| | | D8-E7-A11 | | | | | |
| 3 | 1:1 binding | 2 μg/ml 15- | HUMAN B7H3 | 3.70E−01 | 4.92E+05 | 1.96E−04 | 3.99E−10 |
| | | C8-B5-G7 | | | | | |
| 4 | 1:1 binding | 2 μg/ml 7- | HUMAN B7H3 | 1.97E−01 | 2.60E+05 | 2.22E−04 | 8.55E−10 |
| | | E1-F8-F6 | | | | | |
| 5 | 1:1 binding | 2 μg/ml 27- | HUMAN B7H3 | 2.34E−01 | 2.78E+05 | 1.73E−04 | 6.21E−10 |
| | | F8-E10-E11 | | | | | |
| 6 | 1:1 binding | 2 μg/ml 25- | HUMAN B7H3 | 1.42E−01 | 1.08E+05 | 2.50E−04 | 2.31E−09 |
| | | D3-G4-C6 | | | | | |
| 7 | 1:1 binding | 1 μg/ml 18- | HUMAN B7H3 | 8.59E−01 | 4.89E+05 | 2.39E−04 | 4.89E−10 |
| | | F9-D8-G7 | | | | | |
| 8 | 1:1 binding | 2 μg/ml 20- | HUMAN B7H3 | 8.74E−01 | 5.08E+05 | 2.82E−04 | 5.55E−10 |
| | | A2-D10-G8 | | | | | |
| 9 | 1:1 binding | 2 μg/ml 16- | HUMAN B7H3 | 2.15E+00 | 3.77E+05 | 2.43E−04 | 6.45E−10 |
| | | C6-F7-F5 | | | | | |
| 10 | 1:1 binding | 2 μg/ml 16- | HUMAN B7H3 | 1.29E−01 | 1.85E+05 | 2.14E−04 | 1.16E−09 |
| | | G3-D10-C10 | | | | | |
| 11 | 1:1 binding | 1 μg/ml 10- | HUMAN B7H3 | 1.80E−01 | 2.52E+05 | 1.98E−04 | 7.85E−10 |
| | | G6-C4-B2 | | | | | |
| 12 | 1:1 binding | 1 μg/ml 8- | HUMAN B7H3 | 4.26E−01 | 9.90E+05 | 4.40E−04 | 4.44E−10 |
| | | C3-E3-F3 | | | | | |
| 13 | 1:1 binding | 2 μg/ml 20- | HUMAN B7H3 | 5.33E−01 | 6.45E+05 | 4.16E−04 | 6.44E−10 |
| | | C5-D7-D3 | | | | | |
| 14 | 1:1 binding | 2 μg/ml 25- | HUMAN B7H3 | 3.97E−01 | 2.03E+05 | 2.43E−04 | 1.20E−09 |
| | | C8-D7-C5 | | | | | |
| 15 | 1:1 binding | 1 μg/ml 10- | HUMAN B7H3 | 6.25E−01 | 3.75E+05 | 2.82E−04 | 7.52E−10 |
| | | B9-D10-A12 | | | | | |
| 16 | 1:1 binding | 1 μg/ml | HUMAN B7H3 | 5.98E−01 | 1.28E+05 | 4.86E−04 | 3.80E−09 |
| | | Enoblituzumab | | | | | |
| 17 | 1:1 binding | 2 μg/ml | HUMAN B7H3 | 6.03E−02 | 8.89E+05 | 4.08E−04 | 4.59E−10 |
| | | MGC018 | | | | | |

### 3. B7H3's Expression Pattern on Several Cancer Cell Lines

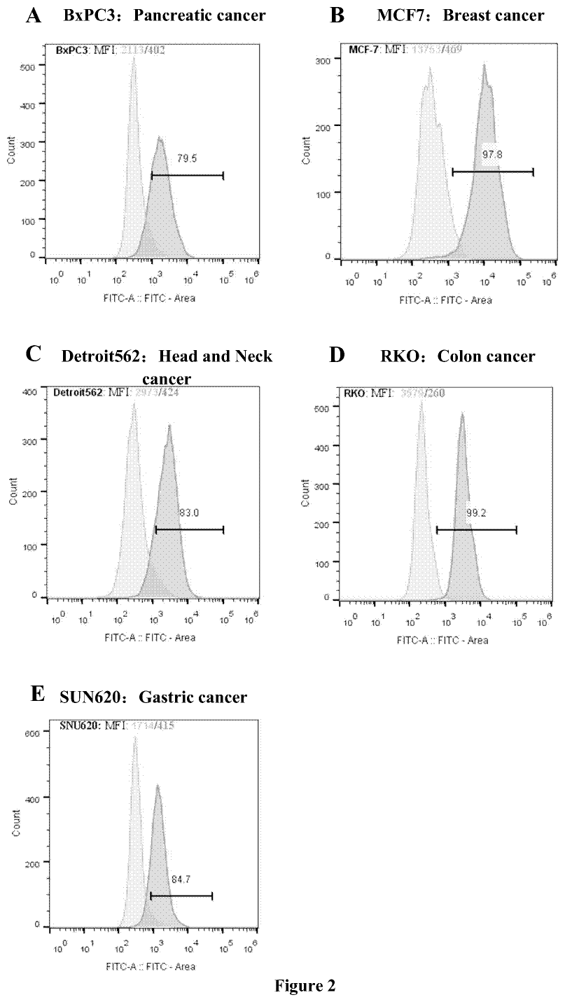

B7H3 expression were detected by FACS with 6-D8-E7-A11. High expression level of B7H3 on several cancer cell lines, such as BxPC3 (Pancreatic), MCF7 (Breast), Dentroit562 (Head and neck), RKO (Colon) and SUN620 (Gastric), were found (see FIG. 2).

### Example 3. Antibody Characterization: ADCC

### 1. Methods

In order to determine the ADCC of the anti-CD276 antibodies, 1×10⁵/well SKOV3 were seeded to 96 wells Flat-bottom sterile plate, then 2×10⁴ Jurkat-NFAT-Luciferase-CD16 were added as effector cell. After that, serial diluted antibodies were added to each well and the plate were incubated at 37° C., 5% CO₂ for 18 hours. Finally, the luciferase activity was detected to evaluate the ADCC activity of antibodies.

### 2. Result

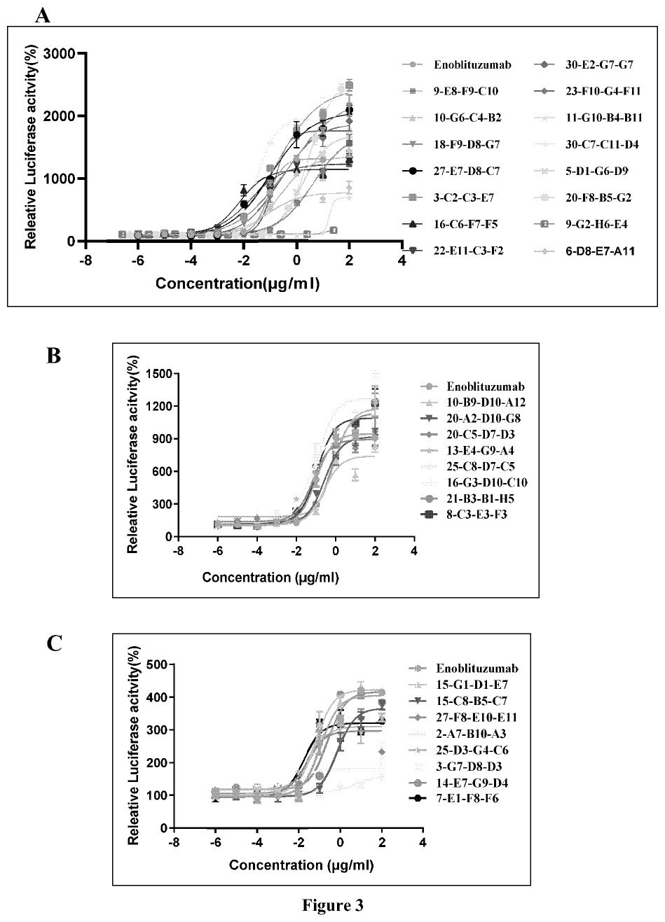

All our antibodies showed potent ADCC effect on SKOV3 cells (a human ovarian cancer cell line). Some of the antibodies showed lower or comparable EC₅₀ compared with bench mark antibody, Enoblituzumab (Table 8 and FIG. 3), indicating that they are more potent in mediating ADCC effect on SKOV3 cells than Enoblituzumab (MGA271).

TABLE 8
EC₅₀ of antibody induced ADCC effect on SKOV3

| | EC₅₀ (μg/ml) |
|---|---|
| Enoblituzumab | 0.08636 |
| 9-E8-F9-C10 | 4.052 |
| 10-G6-C4-B2 | 0.2369 |
| 18-F9-D8-G7 | 0.5032 |
| 27-E7-D8-C7 | 0.1035 |
| 3-C2-C3-E7 | 0.1864 |
| 16-C6-F7-F5 | 0.006641 |
| 22-E11-C3-F2 | 0.01792 |
| 30-E2-G7-G7 | 0.2473 |
| 23-F10-G4-F11 | 0.1367 |
| 11-G10-B4-B11 | 17.34 |
| 30-C7-C11-D4 | 0.03268 |
| 5-D1-G6-D9 | 1.553 |
| 20-F8-B5-G2 | 3.013 |
| 9-G2-H6-E4 | 13.6 |
| 10-B9-D10-A12 | 0.2585 |
| 20-A2-D10-G8 | 0.2625 |
| 20-C5-D7-D3 | 0.06897 |
| 13-E4-G9-A4 | 0.8762 |
| 25-C8-D7-C5 | 0.1228 |
| 16-G3-D10-C10 | 0.04186 |
| 21-B3-B1-H5 | 0.6478 |
| 8-C3-E3-F3 | 0.1111 |
| 15-G1-D1-E3 | 0.06548 |
| 15-C8-B5-G7 | 0.6968 |
| 27-F8-E10-E11 | 0.02385 |
| 25-D3-G4-C6 | 0.03723 |
| 2-A7-B10-A3 | 4.207 |
| 3-G7-D8-D3 | 0.008984 |
| 14-E7-G9-D4 | 0.2516 |
| 7-E1-F8-F6 | 0.01771 |
| 6-D8-E7-A11 | 0.06677 |

### Example 4. Antibody Characterization: CDC

### 1. Methods

In order to determine the CDC of the anti-CD276 antibodies, CHO-S-hCD276 cells were resuspended in cell culture medium at 4E5 cells/mL and were then added into a 96-well opaque wall plate at 50 μL/well. Anti-CD276 antibodies were diluted with complete F-12K medium and added to the 96-well opaque wall plate at 50 μL/well. Human serum complement was diluted with cell culture medium and was added to the same plate at 50 μL/well. The mixture was incubated for 2 hours in a CO₂ incubator at 37° C. CellTiter-Glo reagent for determining the cell cytotoxicity was added at 50 μL/well and the mixture was incubated for 10 mins at R.T. Luminescence signal of viable cells on a microplate reader was recorded.

### 2. Result

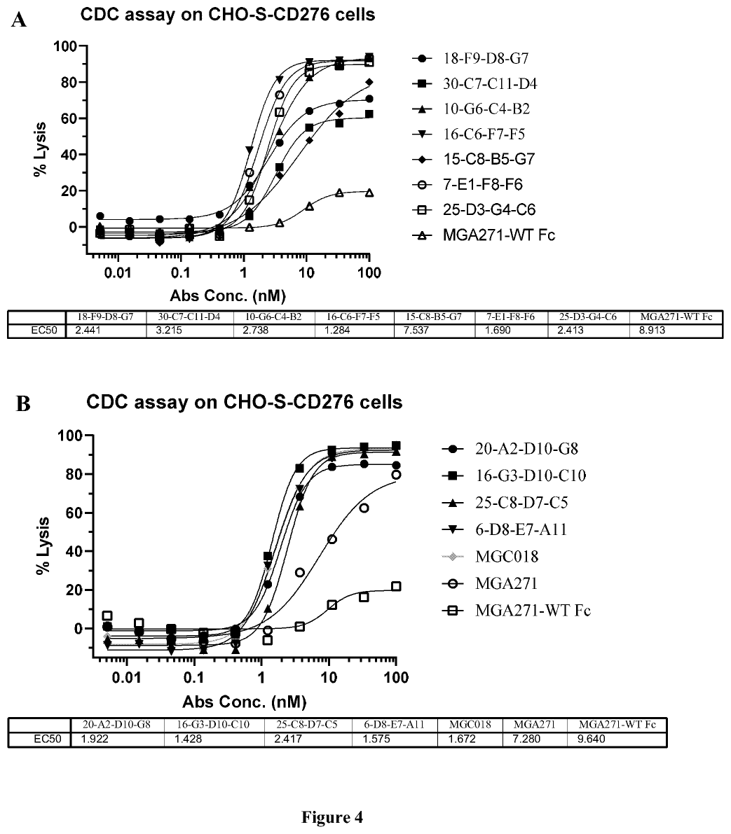

All the anti-CD276 antibodies showed potent CDC effect on CHO-S-hCD276 cells and lower EC₅₀ compared with benchmark antibody, Enoblituzumab (MGA271) (see Table 9 and FIG. 4).

TABLE 9
Max CHO-S-hCD276 killing percentage and
EC₅₀ of antibody induced CDC effect

| | EC₅₀ (nM) | Top cytotoxicity (%) |
|---|---|---|
| 18-F9-D8-G7 | 2.441 | 70.33 |
| 30-C7-C11-D4 | 3.215 | 60.27 |
| 10-G6-C4-B2 | 2.738 | 93.53 |
| 16-C6-F7-F5 | 1.284 | 92.09 |
| 15-C8-B5-G7 | 7.537 | 86.1 |
| 7-E1-F8-F6 | 1.69 | 91.89 |
| 25-D3-G4-C6 | 2.413 | 89.7 |
| MGA271-WT Fc | 8.913 | 19.63 |
| 20-A2-D10-G8 | 1.922 | 85.07 |
| 16-G3-D10-C10 | 1.428 | 93.56 |
| 25-C8-D7-C5 | 2.417 | 91.37 |
| 6-D8-E7-A11 | 1.575 | 92.45 |
| MGC018 | 1.672 | 93.32 |
| MGA271 | 7.28 | 80.09 |
| MGA271-WT Fc | 9.64 | 19.77 |

### Example 5. Antibody Characterization: Indirect ADC Cytotoxicity

### 1. Methods

Fab-ZAP is a chemical conjugate of goat anti-human monovalent antibody (a secondary antibody) and the ribosome-inactivating protein, saporin. Fab-ZAP is used to determine the internalization ability of antibodies. In this assay, 80 μL SKOV-3 cells were plated at 2000 cells/well in a 96-well plate and incubated overnight at 37° C. Anti-CD276 antibodies were then added at 40 μL/well. Fab-ZAP human dilution were added at 40 μL/well and incubated for 96 hours in a CO₂ incubator at 37° C. CellTiter-Glo reagent for determining the cell cytotoxicity were added at 100 μL/well and incubated for 10 mins at R.T. Luminescence signal of viable cells on a microplate reader were recorded.

### 2. Result

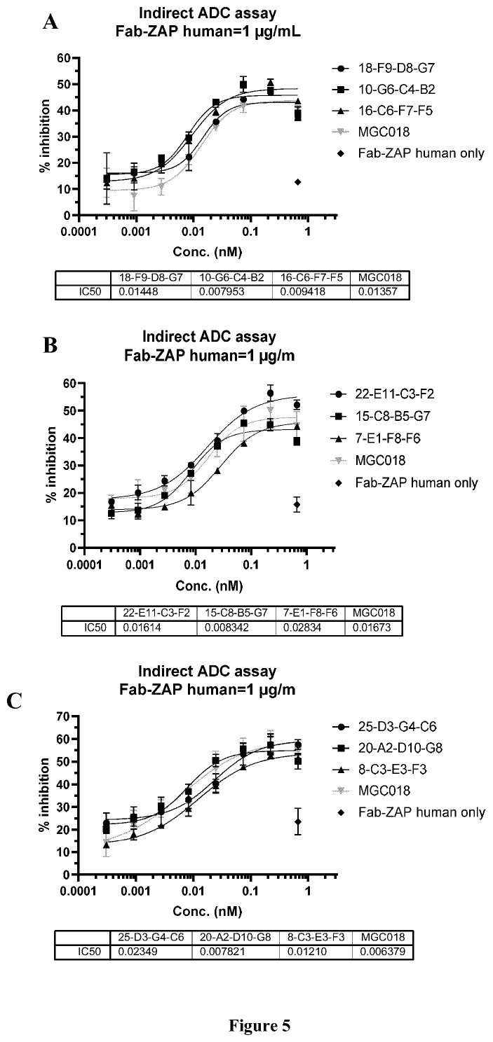

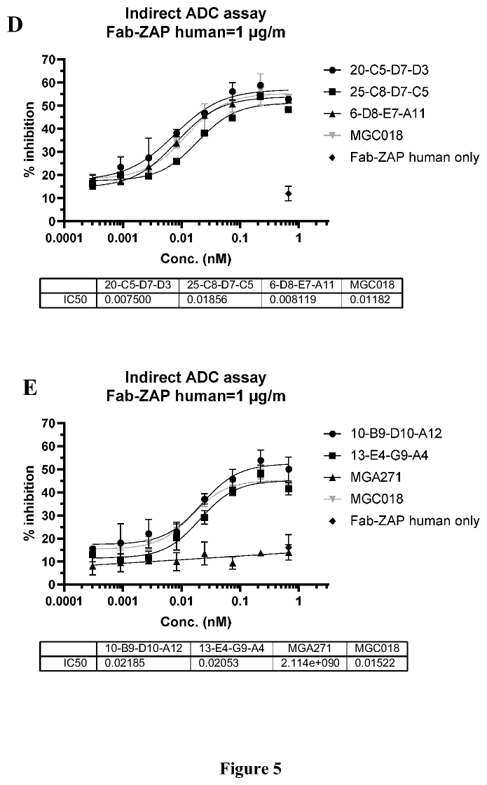

All the anti-CD276 antibodies showed potent indirect ADC effect on SKOV3 cells, with lower or comparable IC₅₀ compared with bench mark antibody, MGC018 (Table 10, FIG. 5), indicating that they are potential candidates for making ADCs.

TABLE 10
IC₅₀ of indirect ADC cytotoxicity on SKOV3

| | IC₅₀ (nM) |
|---|---|
| MGC018 | 0.013 |
| 6-D8-E7-A11 | 0.008 |
| 15-C8-B5-G7 | 0.008 |
| 7-E1-F8-F6 | 0.028 |
| 25-D3-G4-C6 | 0.023 |
| 18-F9-D8-G7 | 0.014 |
| 10-B9-D10-A12 | 0.021 |
| 20-A2-D10-G8 | 0.007 |
| 16-C6-F7-F5 | 0.009 |
| 22-E11-C3-F2 | 0.016 |
| 10-G6-C4-B2 | 0.008 |
| 8-C3-E3-F3 | 0.012 |
| 20-C5-D7-D3 | 0.007 |
| 13-E4-G9-A4 | 0.021 |
| 25-C8-D7-C5 | 0.018 |

### Example 6. Antibody In Vivo Efficacy in the Treatment of Subcutaneous MC-38-hCD276 Murine Colon Carcinoma in Female C57BL/6 Mice

### 1. Study Design and Methods

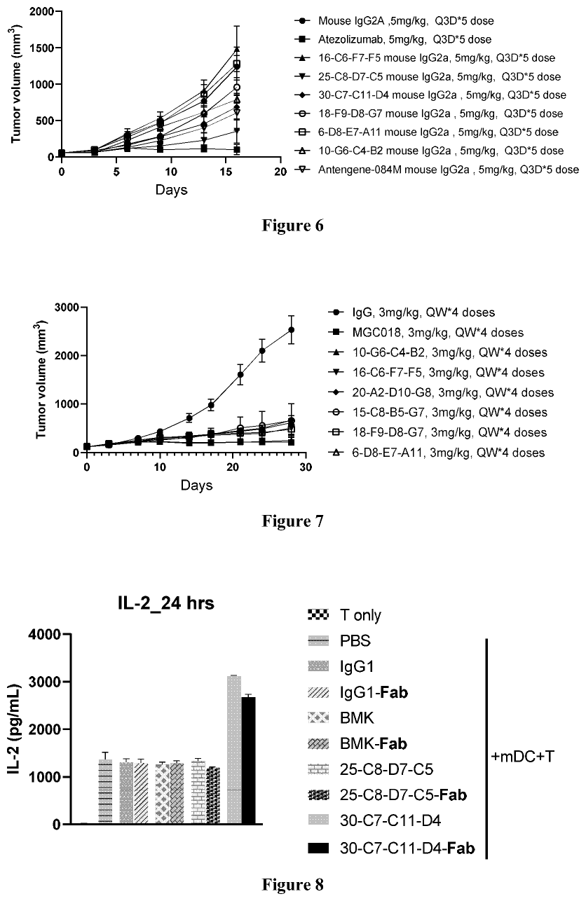

Several monoclonal antibodies with mouse IgG2a Fc were constructed and expressed for the in vivo efficacy study.
1. The MC-38-hCD276 (B7H3) tumor cells were maintained in vitro with DMEM medium supplemented with 10% fetal bovine serum at 37° C. in an atmosphere of 5% CO₂ in air. The cells in exponential growth phase were harvested and quantitated by cell counter before tumor inoculation.
2. Each mouse was inoculated subcutaneously in the right rear flank region with MC-38-hCD276 (B7H3) tumor cells (1×10\hat{ }6) in 0.1 ml of PBS for tumor development. The date of randomization was denoted as day 0, and dosing starts from day 0.
3. The randomization will start when the mean tumor size reaches approximately 50-60 mm³. 60 mice were enrolled in this study. All animals were randomly allocated to 7 study groups. Randomization was performed based on randomized block design.
4. Tumor volumes were measured twice per week in two dimensions using a caliper, and the volume were expressed in mm³ using the formula: V=(L×W×W)/2, where V is tumor volume, L is tumor length (the longest tumor dimension) and W is tumor width (the longest tumor dimension perpendicular to L). Dosing as well as tumor size and body weight measurements were conducted in a Laminar Flow Cabinet (see Table 11, FIG. 6).
5. Atezolizumab (an anti-PD-L1 antibody, also named Tecentriq or MPDL3280A, see WO2010077634A1) and Antengene-084M (MGA271's Fab sequence (see U.S. Pat. No. 8,802,091B) constructed with mouse IgG2a) were used as control.

TABLE 11
Study design for in vivo efficacy test.

| | | | | | Dose level | Dosing | Dosing | | Dosing |
|---|---|---|---|---|---|---|---|---|---|
| | | Mouse | Tumor | | (mg/kg) or | Solution | Volume | | Frequency & |
| Group | No. | Strain | Model | Treatment | (mg/kg) | (mg/ml) | (μL/g) | ROA | Duration |
| 1 | 6 | C57BL/6 | MC-38- | Mouse IgG2A | 5 | 0.5 | 10 | i.p. | Q3D × 5 doses |
| | | | hCD276 | | | | | | |
| 2 | 6 | C57BL/6 | MC-38- | Atezolizumab | 5 | 0.5 | 10 | i.p. | Q3D × 5 doses |
| | | | hCD276 | | | | | | |
| 3 | 6 | C57BL/6 | MC-38- | 16-C6-F7-F5 | 5 | 0.5 | 10 | i.p. | Q3D × 5 doses |
| | | | hCD276 | mouse IgG2a | | | | | |
| 4 | 6 | C57BL/6 | MC-38- | 25-C8-D7-C5 | 5 | 0.5 | 10 | i.p. | Q3D × 5 doses |
| | | | hCD276 | mouse IgG2a | | | | | |
| 5 | 6 | C57BL/6 | MC-38- | 30-C7-C11-D4 | 5 | 0.5 | 10 | i.p. | Q3D × 5 doses |
| | | | hCD276 | mouse IgG2a | | | | | |
| 6 | 6 | C57BL/6 | MC-38- | 18-F9-D8-G7 | 5 | 0.5 | 10 | i.p. | Q3D × 5 doses |
| | | | hCD276 | mouse IgG2a | | | | | |
| 7 | 6 | C57BL/6 | MC-38- | 6-D8-E7-A11 | 5 | 0.5 | 10 | i.p. | Q3D × 5 doses |
| | | | hCD276 | mouse IgG2a | | | | | |
| 8 | 6 | C57BL/6 | MC-38- | 10-G6-C4-B2 | 5 | 0.5 | 10 | i.p. | Q3D × 5 doses |
| | | | hCD276 | mouse IgG2a | | | | | |
| 9 | 6 | C57BL/6 | MC-38- | Antengene- | 5 | 0.5 | 10 | i.p. | Q3D × 5 doses |
| | | | hCD276 | 084M mouse | | | | | |
| | | | | IgG2a | | | | | |

### 2. Result

As shown in FIG. 6, some of the anti-CD276 antibodies showed potent in vivo efficacy. In particular, 25-C8-D7-C5 mouse IgG2a showed higher tumor inhibition than Antengene-084M mouse IgG2a.

### Example 7: ADC In Vivo Efficacy Test of the Antibodies

### 1. Study Design and Methods

To evaluate the ADC potential of the antibodies, antibody conjugations with VC-MMAE in ChemPartner were performed. A serial of VC-MMAE conjugated antibodies were obtained. The details are shown in Table 12.

TABLE 12
Summary of the antibody conjugations

| | Concen- | | SEC | Endotoxin | |
|---|---|---|---|---|---|
| | tration | Qty | Aggregation | level | |
| ADC Name | (mg/mL) | (mg) | (%) | (EU/mg) | HIC-DAR |
| MGC018 | 3.865 | 11.6 | 1.04 | 0.23 | 4.48 |
| 16-C6-F7-F5 | 3.680 | 9.6 | 1.07 | 0.34 | 4.45 |
| 20-A2-D10-G8 | 3.287 | 9.9 | 0.26 | 0.27 | 4.15 |
| 6-D8-E7-A11 | 4.041 | 8.1 | 1.52 | 0.41 | 4.18 |
| 10-G6-C4-B2 | 3.706 | 16.7 | 0.62 | 0.48 | 4.20 |
| 15-C8-B5-G7 | 3.627 | 15.2 | 0.76 | 0.66 | 4.42 |
| 18-F9-D8-G7 | 3.900 | 15.6 | 0.79 | 0.75 | 4.04 |
| higG | 3.825 | 8.0 | 1.09 | 0.29 | 4.39 |

Each Balb/c nude mouse for Calu-6 study was inoculated subcutaneously in the right front flank region with Calu-6 tumor cells (5×10⁶) in 0.1 ml of PBS for tumor development. The randomization starts when the mean tumor size reaches approximately 122 mm³. 60 mice were enrolled in the study. The date of randomization was denoted as day 0, dosing starts from day 0.

### 2. Results

The therapeutic efficacy of test articles in the treatment of subcutaneous human lung cancer Calu-6 in Balb/c nude mice was investigated in this study. No obvious body weight loss, mortality or toxic response was observed in the designed dosing regimens during the efficacy study. At day 24, MGC018, 10-G6-C4-B2, 16-C6-F7-F5, 15-C8-B5-G7 and 18-F9-D8-G7 at 3 mg/kg as a single agent show a significant anti-tumor efficacy against Calu-6 model in Balb/c nude mice, respectively. The results are shown in FIG. 7 and Table 13.

TABLE 13
Antitumor Activity of Test Compounds in Subcutaneous
Calu-6 Model in Balb/c Nude Mice

| | | Tumor Size (mm³) ^{a} | T/C (%) on | TGI (%) on | |
|---|---|---|---|---|---|
| Group | Treatment Description | on day 24 | day 24 | day 24 | P value ^{b} |
| 1 | IgG | 2103.52 ± 236.00 | — | — | — |
| 2 | MGC018 | 215.48 ± 53.71 | 10.24 | 89.76 | <0.0001 |
| 3 | 10-G6-C4-B2 | 229.10 ± 66.60 | 10.89 | 89.11 | <0.0001 |
| 4 | 16-C6-F7-F5 | 398.44 ± 116.04 | 18.94 | 81.06 | 0.00159 |
| 5 | 20-A2-D10-G8 | 504.02 ± 62.98 | 23.96 | 76.04 | 0.0765 |
| 6 | 15-C8-B5-G7 | 561.24 ± 285.12 | 26.68 | 73.32 | 0.00174 |
| 7 | 18-F9-D8-G7 | 418.59 ± 75.48 | 19.90 | 80.10 | 0.0126 |
| 8 | 6-D8-E7-A11 | 493.00 ± 61.89 | 23.44 | 76.56 | 0.0677 |

Note:
^{a} Mean ± SEM;
^{b} TGI, T/C and p values were compared with group 1 tumor volume on day 24 by using Dunnett's tests.

### Example 8: T Cell Activation (MLR Assay) Test of the Antibodies

### 1. Study Design and Methods

### DC Induction

Monocyte cells were re-suspended at 5×10⁶ in 3 ml complete medium supplemented with 2 U/ml of dendritic cell culture factor and then culture cells in 6-well plate. At the second day, 2 ml/well of fresh complete medium supplemented with 2 U/ml of dendritic cell culture factor were added for another three days culture. Then monocytes differentiated into immature dendritic cells (iDC). After stimulated 48 hours with 2 U/ml of dendritic cell mature factor, iDCs would differentiate into mature dendritic cells.

### Antibody Digestion

Combined 25 μg of hIgG1 and 3 μl of 10× Glyco Buffer, and then added PBS to make a 30 μl total reaction volume. 1 μl of IdeZ Protease was added, then incubated at 37° C. for 30 minutes. 10 μl protein A/G beads were added in 1 mL PBS, washed 2 times. The beads were resuspended with reaction solution from the above step.

The tube was put on a magnet for 1 min RT, the F(ab)₂ is in supernatant and the Fc was captured by beads.

### MLR Assay

0.2 million cells/well T cells and 0.02 million cells/well mDCs were seeded.

Cells were treated PBS, IgG1, 25-C8-D7-C5, 30-C7-C11-D4, BMK (MGA271) or their Fab (final concentration 5 μg/ml). After 24 hours, supernatants were collected and human IL-2 and IFNγ was tested with Elisa.

### 2. Results

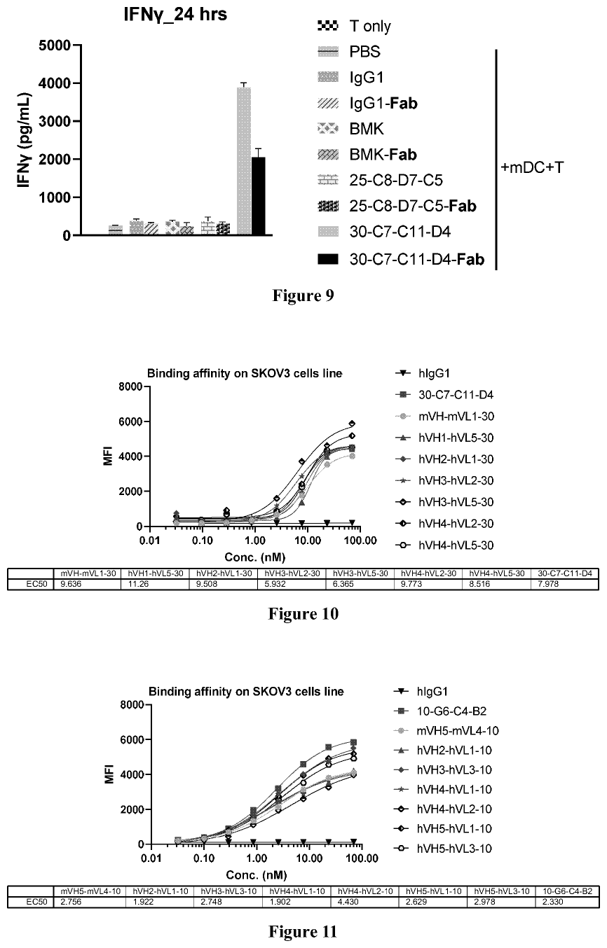

As shown in FIGS. 8 and 9, 30-C7-C11-D4 can activate T cell activation while 25-C8-D7-C5 and BMK (MGA271) cannot.

### Example 9: Antibody Humanization and PTM Optimization

### 9.1 Cell-Based Affinity Test of the Humanized Antibodies by FACS

10-G6-C4-B2 and 30-C7-C11-D4 were chosen to perform humanization and PTM optimization. The affinity of humanized candidates and optimized PTM sequences were evaluated by FACS.

The results are shown in FIGS. 10 and 11. All sequence showed comparable affinity with parental antibody.

### 9.2 Protein Based Affinity Test by Biacore (ChemPartner)

### Test Condition:

Analyte: B7H3. Running buffer: HBS-EP+. Flow Rate: 30 L/min. Capture: Abs, 10 μL/min for 60 s. Injection of serial diluted B7H3. Contact time 180 s, dissociation time 400 s. Regeneration: pH1.5 Gly, 30 μL/min for 30 s. Method: Multiple cycle kinetics/affinity using capture. Machine Model: Biacore 8K (GE). Analysis Temperature: 25° C.

The results are shown in Table 14.

TABLE 14
Binding affinity of the humanized antibodies on hB7H3 by
Biacore (ChemPartner)

| Kinetics | | Analyte 1 | Kinetics | | | |
|---|---|---|---|---|---|---|
| model | Capture 1 Solution | Solution | Chi² (RU²) | ka (1/Ms) | kd (1/s) | KD (M) |
| 1:1 binding | 10-G6-C4-B2 | B7H3 | 2.05E−01 | 2.16E+05 | 8.97E−05 | 4.15E−10 |
| 1:1 binding | 10-G6-C4-B2_hVH2- | B7H3 | 1.10E−01 | 8.53E+04 | 7.26E−05 | 8.51E−10 |
| | VL_1_PTM | | | | | |
| 1:1 binding | 10-G6-C4-B2_hVH3- | B7H3 | 1.61E−01 | 1.18E+05 | 1.02E−06 | 8.70E−12 |
| | VL3_PTM | | | | | |
| 1:1 binding | 10-G6-C4-B2_h_VH4- | B7H3 | 1.16E−01 | 8.96E+04 | 1.55E−04 | 1.73E−09 |
| | VL1_PTM | | | | | |
| 1:1 binding | 10-G6-C4-B2_hVH4- | B7H3 | 1.21E−01 | 5.27E+04 | 7.12E−05 | 1.35E−09 |
| | VL2_PTM | | | | | |
| 1:1 binding | 10-G6-C4-B2_hVH5- | B7H3 | 1.11E−01 | 1.47E+05 | 1.37E−05 | 9.29E−11 |
| | VL_1_PTM | | | | | |
| 1:1 binding | 10-G6-C4-B2_hVH5- | B7H3 | 2.61E−01 | 1.46E+05 | 1.01E−06 | 6.92E−12 |
| | VL3_PTM | | | | | |

### 9.3 ADC Therapeutic Capability Test of the Humanized 10-G6-C4-B2 Antibodies In Vivo

The in vivo ADC therapeutic capability of the humanized 10-G6-C4-B2 antibodies were evaluated. Antibody conjugations with VC-MMAE in ChemPartner were constructed. A serial of VC-MMAE conjugated antibodies were obtained, the details of which were shown in Table 15.

TABLE 15
Summary of the antibody conjugations

| | | | SEC | | |
|---|---|---|---|---|---|
| | Concen- | | Aggre- | | Endotoxin |
| | tration | Qty | gation | HIC- | Level |
| ADC Name | (mg/mL) | (mg) | (%) | DAR | (EU/mg) |
| 10-G6-C4-B2_h_VH2- | 3.073 | 6.88 | 0.85 | 4.69 | 0.039 |
| VL1_PTM | | | | | |
| 10-G6-C4-B2_hVH3- | 3.186 | 8.19 | 0.5 | 4.44 | 0.028 |
| VL3_PTM | | | | | |
| 10-G6-C4-B2_hVH4- | 3.181 | 7.60 | 1.37 | 4.57 | 0.047 |
| VL1_PTM | | | | | |
| 10-G6-C4-B2_hVH4- | 2.950 | 7.82 | 1.82 | 4.22 | 0.041 |
| VL2_PTM | | | | | |
| 10-G6-C4-B2_hVH5- | 1.834 | 1.47 | 1.56 | 4.33 | Not tested |
| VL1_PTM | | | | | |
| 10-G6-C4-B2_hVH5- | 3.251 | 7.90 | 0.2 | 4.6 | 0.055 |
| VL3_PTM | | | | | |
| IgG | 3.286 | 7.85 | 1.19 | 4.63 | 0.037 |
| 10-G6-C4-B2 | 2.506 | 4.21 | 0.43 | 4.35 | 0.036 |

Each Balb/c nude mouse for Calu-6 study was inoculated subcutaneously in the right front flank region with Calu-6 tumor cells (5×10⁶) in 0.1 ml of PBS for tumor development. The randomization starts when the mean tumor size reaches approximately 122 mm³. 60 mice were enrolled in the study. The date of randomization was denoted as day 0, dosing starts from day 0.

The therapeutic efficacy of test articles in the treatment of subcutaneous human lung cancer Calu-6 in Balb/c nude mice was investigated in this study. No obvious body weight loss, mortality or toxic response was observed in the designed dosing regimens during the efficacy study.

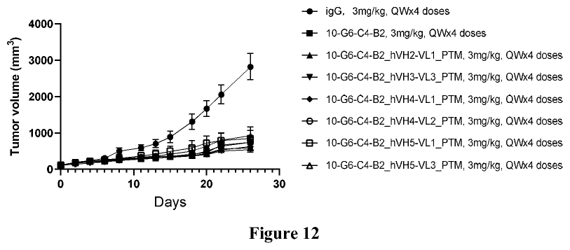

The results are shown in FIG. 12 and Table 16. At day 26, almost all the test articles at 3 mg/kg as a single agent show a significant anti-tumor efficacy against Calu-6 model in Balb/c nude mice, respectively.

TABLE 16
Antitumor activity of test compounds in subcutaneous
Calu-6 model in Balb/c nude mice

| | | T/C | TGI | |
|---|---|---|---|---|
| | Tumor Size (mm³) ^{a} | (%) on | (%) on | |
| Treatment Description | on day 26 | day 26 | day 26 | P value ^{b} |
| igG, 3 mg/kg, QWx4 doses | 2823.12 ± 361.18(6) | — | — | — |
| 10-G6-C4-B2, 3 mg/kg, QWx4 | 752.77 ± 33.24(6) | 26.66 | 73.34 | >0.05 |
| doses | | | | (0.0645) |
| 10-G6-C4-B2_hVH2- | 936.63 ± 227.54(6) | 33.18 | 66.82 | <0.05 |
| VL1_PTM, 3 mg/kg, QWx4 | | | | (0.0421) |
| doses | | | | |
| 10-G6-C4-B2_hVH3- | 544.41 ± 49.50(6) | 19.28 | 80.72 | <0.001 |
| VL3_PTM, 3 mg/kg, QWx4 | | | | (4.35E−05) |
| doses | | | | |
| 10-G6-C4-B2_hVH4- | 633.69 ± 61.73(6) | 22.45 | 77.55 | <0.001 |
| VL1_PTM, 3 mg/kg, QWx4 | | | | (0.000727) |
| doses | | | | |
| 10-G6-C4-B2_hVH4- | 744.98 ± 69.83(6) | 26.39 | 73.61 | <0.05 |
| VL2_PTM, 3 mg/kg, QWx4 | | | | (0.0396) |
| doses | | | | |
| 10-G6-C4-B2_hVH5- | 856.17 ± 216.11(6) | 30.33 | 69.67 | <0.01 |
| VL1_PTM, 3 mg/kg, QWx4 | | | | (0.00691) |
| doses | | | | |
| 10-G6-C4-B2_hVH5- | 595.32 ± 124.15(6) | 21.09 | 78.91 | <0.001 |
| VL3_PTM, 3 mg/kg, QWx4 | | | | (0.000593) |
| doses | | | | |

Note:
Mean ± SEM; TGI, T/C and p values were compared with group 1 tumor volume on day 26 using Kruskal-Wallis test. . .

1-3. (canceled)

4. An antibody or antigen-binding fragment thereof, comprising:
a) a heavy chain variable region comprising 1, 2, or 3 CDR sequences selected from SEQ ID NO: 1, SEQ ID NO: 2, and SEQ ID NO: 3; and a light chain variable region comprising 1, 2, or 3 CDR sequences selected from SEQ ID NO: 4, SEQ ID NO: 5, and SEQ ID NO: 6;
b) a heavy chain variable region comprising 1, 2, or 3 CDR sequences selected from SEQ ID NO: 9, SEQ ID NO: 10, and SEQ ID NO: 11; and a light chain variable region comprising 1, 2, or 3 CDR sequences selected from SEQ ID NO: 12, SEQ ID NO: 13, and SEQ ID NO: 14;
c) a heavy chain variable region comprising 1, 2, or 3 CDR sequences selected from SEQ ID NO: 17, SEQ ID NO: 18, and SEQ ID NO: 19; and a light chain variable region comprising 1, 2, or 3 CDR sequences selected from SEQ ID NO: 20, SEQ ID NO: 21 and SEQ ID NO: 22;
d) a heavy chain variable region comprising 1, 2, or 3 CDR sequences selected from SEQ ID NO: 25, SEQ ID NO: 26, and SEQ ID NO: 27; and a light chain variable region comprising 1, 2, or 3 CDR sequences selected from SEQ ID NO: 28, SEQ ID NO: 29, and SEQ ID NO: 30;
e) a heavy chain variable region comprising 1, 2, or 3 CDR sequences selected from SEQ ID NO: 33, SEQ ID NO: 34, and SEQ ID NO: 35; and a light chain variable region comprising 1, 2, or 3 CDR sequences selected from SEQ ID NO: 36, SEQ ID NO: 37, and SEQ ID NO: 38;
f) a heavy chain variable region comprising 1, 2, or 3 CDR sequences selected from SEQ ID NO:41, SEQ ID NO: 42, and SEQ ID NO: 43; and a light chain variable region comprising 1, 2, or 3 CDR sequences selected from SEQ ID NO: 44, SEQ ID NO: 45, and SEQ ID NO:46;
g) a heavy chain variable region comprising 1, 2, or 3 CDR sequences selected from SEQ ID NO:49, SEQ ID NO: 50, and SEQ ID NO: 51; and a light chain variable region comprising 1, 2, or 3 CDR sequences selected from SEQ ID NO: 52, SEQ ID NO: 53, and SEQ ID NO: 54;
h) a heavy chain variable region comprising 1, 2, or 3 CDR sequences selected from SEQ ID NO: 57, SEQ ID NO: 58, and SEQ ID NO: 59; and a light chain variable region comprising 1, 2, or 3 CDR sequences selected from SEQ ID SEQ ID NO: 60, SEQ ID NO: 61, and SEQ ID NO: 62;
i) a heavy chain variable region comprising 1, 2, or 3 CDR sequences selected from SEQ ID NO: 65, SEQ ID NO: 66, and SEQ ID NO: 67; and a kappa light chain variable region comprising 1, 2, or 3 CDR sequences selected from SEQ ID NO: 68, SEQ ID NO: 69, and SEQ ID NO: 70;
j) a heavy chain variable region comprising 1, 2, or 3 CDR sequences selected from SEQ ID NO: 73, SEQ ID NO: 74, and SEQ ID NO: 75; and a kappa light chain variable region comprising 1, 2, or 3 CDR sequences selected from SEQ ID NO: 76, SEQ ID NO: 77, and SEQ ID NO: 78;
k) a heavy chain variable region comprising 1, 2, or 3 CDR sequences selected from SEQ ID NO: 81, SEQ ID NO: 82, and SEQ ID NO: 83; and a kappa light chain variable region comprising 1, 2, or 3 CDR sequences selected from SEQ ID NO: 84, SEQ ID NO: 85, and SEQ ID NO: 86;
l) a heavy chain variable region comprising 1, 2, or 3 CDR sequences selected from SEQ ID NO: 89, SEQ ID NO: 90, and SEQ ID NO: 91; and a light chain variable region comprising 1, 2, or 3 CDR sequences selected from SEQ ID NO: 92, SEQ ID NO: 93, and SEQ ID NO: 94;
m) a heavy chain variable region comprising 1, 2, or 3 CDR sequences selected from SEQ ID NO: 97, SEQ ID NO: 98, and SEQ ID NO: 99; and a light chain variable region comprising 1, 2, or 3 CDR sequences selected from SEQ ID NO: 100, SEQ ID NO: 101, and SEQ ID NO: 102;
n) a heavy chain variable region comprising 1, 2, or 3 CDR sequences selected from SEQ ID NO: 105, SEQ ID NO: 106, and SEQ ID NO: 107; and a light chain variable region comprising 1, 2, or 3 CDR sequences selected from SEQ ID NO: 108, SEQ ID NO: 109 and SEQ ID NO: 110;
o) a heavy chain variable region comprising 1, 2, or 3 CDR sequences selected from SEQ ID NO: 113, SEQ ID NO: 114, and SEQ ID NO: 115; and a light chain variable region comprising 1, 2, or 3 CDR sequences selected from SEQ ID NO: 116, SEQ ID NO: 117, and SEQ ID NO: 118;
p) a heavy chain variable region comprising 1, 2, or 3 CDR sequences selected from SEQ ID NO: 121, SEQ ID NO: 122, and SEQ ID NO: 123; and a light chain variable region comprising 1, 2, or 3 CDR sequences selected from SEQ ID NO: 124, SEQ ID NO: 125, and SEQ ID NO: 126;
q) a heavy chain variable region comprising 1, 2, or 3 CDR sequences selected from SEQ ID NO:129, SEQ ID NO: 130, and SEQ ID NO: 131; and a light chain variable region comprising 1, 2, or 3 CDR sequences selected from SEQ ID NO: 132, SEQ ID NO: 133, and SEQ ID NO:134;
r) a heavy chain variable region comprising 1, 2, or 3 CDR sequences selected from SEQ ID NO:137, SEQ ID NO: 138, and SEQ ID NO: 139; and a light chain variable region comprising 1, 2, or 3 CDR sequences selected from SEQ ID NO: 140, SEQ ID NO: 141, and SEQ ID NO: 142;
s) a heavy chain variable region comprising 1, 2, or 3 CDR sequences selected from SEQ ID NO: 145, SEQ ID NO: 146, and SEQ ID NO: 147; and a light chain variable region comprising 1, 2, or 3 CDR sequences selected from SEQ ID SEQ ID NO: 148, SEQ ID NO: 149, and SEQ ID NO: 150;
t) a heavy chain variable region comprising 1, 2, or 3 CDR sequences selected from SEQ ID NO: 153, SEQ ID NO: 154, and SEQ ID NO: 155; and a kappa light chain variable region comprising 1, 2, or 3 CDR sequences selected from SEQ ID NO: 156, SEQ ID NO: 157, and SEQ ID NO: 158;
u) a heavy chain variable region comprising 1, 2, or 3 CDR sequences selected from SEQ ID NO: 161, SEQ ID NO: 162, and SEQ ID NO: 163; and a kappa light chain variable region comprising 1, 2, or 3 CDR sequences selected from SEQ ID NO: 164, SEQ ID NO: 165, and SEQ ID NO: 166;
v) a heavy chain variable region comprising 1, 2, or 3 CDR sequences selected from SEQ ID NO: 169, SEQ ID NO: 170, and SEQ ID NO: 171; and a kappa light chain variable region comprising 1, 2, or 3 CDR sequences selected from SEQ ID NO: 172, SEQ ID NO: 173, and SEQ ID NO: 174;
w) a heavy chain variable region comprising 1, 2, or 3 CDR sequences selected from SEQ ID NO: 177, SEQ ID NO: 178, and SEQ ID NO: 179; and a light chain variable region comprising 1, 2, or 3 CDR sequences selected from SEQ ID NO: 180, SEQ ID NO: 181, and SEQ ID NO: 182;
x) a heavy chain variable region comprising 1, 2, or 3 CDR sequences selected from SEQ ID NO: 185, SEQ ID NO: 186, and SEQ ID NO: 187; and a light chain variable region comprising 1, 2, or 3 CDR sequences selected from SEQ ID NO: 188, SEQ ID NO: 189, and SEQ ID NO: 190;
y) a heavy chain variable region comprising 1, 2, or 3 CDR sequences selected from SEQ ID NO: 193, SEQ ID NO: 194, and SEQ ID NO: 195; and a light chain variable region comprising 1, 2, or 3 CDR sequences selected from SEQ ID NO: 196, SEQ ID NO: 197 and SEQ ID NO: 198;
z) a heavy chain variable region comprising 1, 2, or 3 CDR sequences selected from SEQ ID NO: 201, SEQ ID NO: 202, and SEQ ID NO: 203; and a light chain variable region comprising 1, 2, or 3 CDR sequences selected from SEQ ID NO: 204, SEQ ID NO: 205, and SEQ ID NO: 206;
aa) a heavy chain variable region comprising 1, 2, or 3 CDR sequences selected from SEQ ID NO: 209, SEQ ID NO: 210, and SEQ ID NO: 211; and a light chain variable region comprising 1, 2, or 3 CDR sequences selected from SEQ ID NO: 212, SEQ ID NO: 213, and SEQ ID NO: 214;
bb) a heavy chain variable region comprising 1, 2, or 3 CDR sequences selected from SEQ ID NO:217, SEQ ID NO: 218, and SEQ ID NO: 219; and a light chain variable region comprising 1, 2, or 3 CDR sequences selected from SEQ ID NO: 220, SEQ ID NO: 221, and SEQ ID NO:222;
cc) a heavy chain variable region comprising 1, 2, or 3 CDR sequences selected from SEQ ID NO:225, SEQ ID NO: 226, and SEQ ID NO: 227; and a light chain variable region comprising 1, 2, or 3 CDR sequences selected from SEQ ID NO: 228, SEQ ID NO: 229, and SEQ ID NO: 230;
dd) a heavy chain variable region comprising 1, 2, or 3 CDR sequences selected from SEQ ID NO: 233, SEQ ID NO: 234, and SEQ ID NO: 235; and a light chain variable region comprising 1, 2, or 3 CDR sequences selected from SEQ ID SEQ ID NO: 236, SEQ ID NO: 237, and SEQ ID NO: 238;
ee) a heavy chain variable region comprising 1, 2, or 3 CDR sequences selected from SEQ ID NO: 241, SEQ ID NO: 242, and SEQ ID NO: 243; and a kappa light chain variable region comprising 1, 2, or 3 CDR sequences selected from SEQ ID NO: 244, SEQ ID NO: 245, and SEQ ID NO: 246;
ff) a heavy chain variable region comprising 1, 2, or 3 CDR sequences selected from SEQ ID NO: 249, SEQ ID NO: 250, and SEQ ID NO: 251; and a kappa light chain variable region comprising 1, 2, or 3 CDR sequences selected from SEQ ID NO: 252, SEQ ID NO: 253, and SEQ ID NO: 254;
gg) a heavy chain variable region comprising 1, 2, or 3 CDR sequences selected from SEQ ID NO: 257, SEQ ID NO: 258, and SEQ ID NO: 259; and a kappa light chain variable region comprising 1, 2, or 3 CDR sequences selected from SEQ ID NO: 260, SEQ ID NO: 261, and SEQ ID NO: 262;
hh) a heavy chain variable region comprising 1, 2, or 3 CDR sequences selected from SEQ ID NO: 265, SEQ ID NO: 266, and SEQ ID NO: 267; and a light chain variable region comprising 1, 2, or 3 CDR sequences selected from SEQ ID NO: 268, SEQ ID NO: 269, and SEQ ID NO: 270;
ii) a heavy chain variable region comprising 1, 2, or 3 CDR sequences selected from SEQ ID NO: 273, SEQ ID NO: 274, and SEQ ID NO: 275; and a light chain variable region comprising 1, 2, or 3 CDR sequences selected from SEQ ID NO: 276, SEQ ID NO: 277, and SEQ ID NO: 278;
jj) a heavy chain variable region comprising 1, 2, or 3 CDR sequences selected from SEQ ID NO: 281, SEQ ID NO: 282, and SEQ ID NO: 283; and a light chain variable region comprising 1, 2, or 3 CDR sequences selected from SEQ ID NO: 284, SEQ ID NO: 285 and SEQ ID NO: 286;
kk) a heavy chain variable region comprising 1, 2, or 3 CDR sequences selected from SEQ ID NO: 289, SEQ ID NO: 290, and SEQ ID NO: 291; and a light chain variable region comprising 1, 2, or 3 CDR sequences selected from SEQ ID NO: 292, SEQ ID NO: 293, and SEQ ID NO: 294;
ll) a heavy chain variable region comprising 1, 2, or 3 CDR sequences selected from SEQ ID NO: 297, SEQ ID NO: 298, and SEQ ID NO: 299; and a light chain variable region comprising 1, 2, or 3 CDR sequences selected from SEQ ID NO: 300, SEQ ID NO: 301, and SEQ ID NO: 302;
mm) a heavy chain variable region comprising 1, 2, or 3 CDR sequences selected from SEQ ID NO:305, SEQ ID NO: 306, and SEQ ID NO: 307; and a light chain variable region comprising 1, 2, or 3 CDR sequences selected from SEQ ID NO: 308, SEQ ID NO: 309, and SEQ ID NO:310;
nn) a heavy chain variable region comprising 1, 2, or 3 CDR sequences selected from SEQ ID NO:313, SEQ ID NO: 314, and SEQ ID NO: 315; and a light chain variable region comprising 1, 2, or 3 CDR sequences selected from SEQ ID NO: 316, SEQ ID NO: 317, and SEQ ID NO: 318;
oo) a heavy chain variable region comprising 1, 2, or 3 CDR sequences selected from SEQ ID NO: 321, SEQ ID NO: 322, and SEQ ID NO: 323; and a light chain variable region comprising 1, 2, or 3 CDR sequences selected from SEQ ID SEQ ID NO: 324, SEQ ID NO: 325, and SEQ ID NO: 326;
pp) a heavy chain variable region comprising 1, 2, or 3 CDR sequences selected from SEQ ID NO: 329, SEQ ID NO: 330, and SEQ ID NO: 331; and a kappa light chain variable region comprising 1, 2, or 3 CDR sequences selected from SEQ ID NO: 332, SEQ ID NO: 333, and SEQ ID NO: 334;
qq) a heavy chain variable region comprising 1, 2, or 3 CDR sequences selected from SEQ ID NO: 337, SEQ ID NO: 338, and SEQ ID NO: 339; and a kappa light chain variable region comprising 1, 2, or 3 CDR sequences selected from SEQ ID NO: 340, SEQ ID NO: 341, and SEQ ID NO: 342;
rr) a heavy chain variable region comprising 1, 2, or 3 CDR sequences selected from SEQ ID NO: 9, SEQ ID NO: 374, and SEQ ID NO: 375; and a kappa light chain variable region comprising 1, 2, or 3 CDR sequences selected from SEQ ID NO: 376, SEQ ID NO: 13, and SEQ ID NO: 14; or
ss) a heavy chain variable region comprising 1, 2, or 3 CDR sequences selected from SEQ ID NO: 41, SEQ ID NO: 42, and SEQ ID NO: 43; and a kappa light chain variable region comprising 1, 2, or 3 CDR sequences selected from SEQ ID NO: 377, SEQ ID NO: 45, and SEQ ID NO: 46.

5. The antibody or antigen-binding fragment thereof of claim 4, comprising a heavy chain variable region selected from the group consisting of: SEQ ID NO: 7, SEQ ID NO: 15, SEQ ID NO: 23, SEQ ID NO: 31, SEQ ID NO: 39, SEQ ID NO: 47, SEQ ID NO: 55, SEQ ID NO: 63, SEQ ID NO: 71, SEQ ID NO: 79, SEQ ID NO: 87, SEQ ID NO: 95, SEQ ID NO: 103, SEQ ID NO: 111, SEQ ID NO: 119, SEQ ID NO: 127, SEQ ID NO: 135, SEQ ID NO: 143, SEQ ID NO: 151, SEQ ID NO: 159, SEQ ID NO: 167, SEQ ID NO: 175, SEQ ID NO: 183, SEQ ID NO: 191, SEQ ID NO: 199, SEQ ID NO: 207, SEQ ID NO: 215, SEQ ID NO: 223, SEQ ID NO: 231, SEQ ID NO: 239, SEQ ID NO: 247, SEQ ID NO: 255, SEQ ID NO: 263, SEQ ID NO: 271, SEQ ID NO: 279, SEQ ID NO: 287, SEQ ID NO: 295, SEQ ID NO: 303, SEQ ID NO: 311, SEQ ID NO: 319, SEQ ID NO: 327, SEQ ID NO: 335, SEQ ID NO: 343, SEQ ID NO: 347, and SEQ ID NO: 349 and the homologue sequences of at least 80% sequence identity thereof.

6. The antibody or antigen-binding fragment thereof of claim 4, comprising a light chain variable region selected from the group consisting of: SEQ ID NO: 8, SEQ ID NO: 16, SEQ ID NO: 24, SEQ ID NO: 32, SEQ ID NO: 40, SEQ ID NO: 48, SEQ ID NO: 56, SEQ ID NO: 64, SEQ ID NO: 72, SEQ ID NO: 80, SEQ ID NO: 88, SEQ ID NO: 96, SEQ ID NO: 104, SEQ ID NO: 112, SEQ ID NO: 120, SEQ ID NO: 128, SEQ ID NO: 136, SEQ ID NO: 144, SEQ ID NO: 152, SEQ ID NO: 160, SEQ ID NO: 168, SEQ ID NO: 176, SEQ ID NO: 184, SEQ ID NO: 192, SEQ ID NO: 200, SEQ ID NO: 208, SEQ ID NO: 216, SEQ ID NO: 224, SEQ ID NO: 232, SEQ ID NO: 240, SEQ ID NO: 248, SEQ ID NO: 256, SEQ ID NO: 264, SEQ ID NO: 272, SEQ ID NO: 280, SEQ ID NO: 288, SEQ ID NO: 296, SEQ ID NO: 304, SEQ ID NO: 312, SEQ ID NO: 320, SEQ ID NO: 328, SEQ ID NO: 336, SEQ ID NO: 344, SEQ ID NO: 348, and SEQ ID NO: 350 and the homologue sequences of at least 80% sequence identity thereof.

7. The antibody or antigen-binding fragment thereof of claim 4, comprising:
a) a heavy chain variable region comprising SEQ ID NO: 7 and a light chain variable region comprising SEQ ID NO: 8;
b) a heavy chain variable region comprising SEQ ID NO: 15 and a light chain variable region comprising SEQ ID NO: 16;
c) a heavy chain variable region comprising SEQ ID NO: 23 and a light chain variable region comprising SEQ ID NO: 24;
d) a heavy chain variable region comprising SEQ ID NO: 31 and a light chain variable region comprising SEQ ID NO: 32;
e) a heavy chain variable region comprising SEQ ID NO: 39 and a light chain variable region comprising SEQ ID NO: 40;
f) a heavy chain variable region comprising SEQ ID NO: 47 and a light chain variable region comprising SEQ ID NO: 48;
g) a heavy chain variable region comprising SEQ ID NO: 55 and a light chain variable region comprising SEQ ID NO: 56;
h) a heavy chain variable region comprising SEQ ID NO: 63 and a light chain variable region comprising SEQ ID NO: 64;
i) a heavy chain variable region comprising SEQ ID NO: 71 and a light chain variable region comprising SEQ ID NO: 72;
j) a heavy chain variable region comprising SEQ ID NO: 79 and a light chain variable region comprising SEQ ID NO: 80;
k) a heavy chain variable region comprising SEQ ID NO: 87 and a light chain variable region comprising SEQ ID NO: 88;
l) a heavy chain variable region comprising SEQ ID NO: 95 and a light chain variable region comprising SEQ ID NO: 96;
m) a heavy chain variable region comprising SEQ ID NO: 103 and a light chain variable region comprising SEQ ID NO: 104;
n) a heavy chain variable region comprising SEQ ID NO: 111 and a light chain variable region comprising SEQ ID NO: 112;
o) a heavy chain variable region comprising SEQ ID NO: 119 and a light chain variable region comprising SEQ ID NO: 120;
p) a heavy chain variable region comprising SEQ ID NO: 127 and a light chain variable region comprising SEQ ID NO: 128;
q) a heavy chain variable region comprising SEQ ID NO: 135 and a light chain variable region comprising SEQ ID NO: 136;
r) a heavy chain variable region comprising SEQ ID NO: 143 and a light chain variable region comprising SEQ ID NO: 144;
s) a heavy chain variable region comprising SEQ ID NO: 151 and a light chain variable region comprising SEQ ID NO: 152;
t) a heavy chain variable region comprising SEQ ID NO: 159 and a light chain variable region comprising SEQ ID NO: 160;
u) a heavy chain variable region comprising SEQ ID NO: 167 and a light chain variable region comprising SEQ ID NO: 168;
v) a heavy chain variable region comprising SEQ ID NO: 175 and a light chain variable region comprising SEQ ID NO: 176;
w) a heavy chain variable region comprising SEQ ID NO: 183 and a light chain variable region comprising SEQ ID NO: 184;
x) a heavy chain variable region comprising SEQ ID NO: 191 and a light chain variable region comprising SEQ ID NO: 192;
y) a heavy chain variable region comprising SEQ ID NO: 199 and a light chain variable region comprising SEQ ID NO: 200;
z) a heavy chain variable region comprising SEQ ID NO: 207 and a light chain variable region comprising SEQ ID NO: 208;
aa) a heavy chain variable region comprising SEQ ID NO: 215 and a light chain variable region comprising SEQ ID NO: 216;
bb) a heavy chain variable region comprising SEQ ID NO: 223 and a light chain variable region comprising SEQ ID NO: 224;
cc) a heavy chain variable region comprising SEQ ID NO: 231 and a light chain variable region comprising SEQ ID NO: 232;
dd) a heavy chain variable region comprising SEQ ID NO: 239 and a light chain variable region comprising SEQ ID NO: 240;
ee) a heavy chain variable region comprising SEQ ID NO: 247 and a light chain variable region comprising SEQ ID NO: 248;
ff) a heavy chain variable region comprising SEQ ID NO: 255 and a light chain variable region comprising SEQ ID NO: 256;
gg) a heavy chain variable region comprising SEQ ID NO: 263 and a light chain variable region comprising SEQ ID NO: 264;
hh) a heavy chain variable region comprising SEQ ID NO: 271 and a light chain variable region comprising SEQ ID NO: 272;
ii) a heavy chain variable region comprising SEQ ID NO: 279 and a light chain variable region comprising SEQ ID NO: 280;
jj) a heavy chain variable region comprising SEQ ID NO: 287 and a light chain variable region comprising SEQ ID NO: 288;
kk) a heavy chain variable region comprising SEQ ID NO: 295 and a light chain variable region comprising SEQ ID NO: 296;
ll) a heavy chain variable region comprising SEQ ID NO: 303 and a light chain variable region comprising SEQ ID NO: 304;
mm) a heavy chain variable region comprising SEQ ID NO: 311 and a light chain variable region comprising SEQ ID NO: 312;
nn) a heavy chain variable region comprising SEQ ID NO: 319 and a light chain variable region comprising SEQ ID NO: 320;
oo) a heavy chain variable region comprising SEQ ID NO: 327 and a light chain variable region comprising SEQ ID NO: 328;
pp) a heavy chain variable region comprising SEQ ID NO: 335 and a light chain variable region comprising SEQ ID NO:336;
qq) a heavy chain variable region comprising SEQ ID NO: 343 and a light chain variable region comprising SEQ ID NO: 344;
rr) a heavy chain variable region comprising SEQ ID NO: 347 and a light chain variable region comprising SEQ ID NO: 348; or
ss) a heavy chain variable region comprising SEQ ID NO: 349 and a light chain variable region comprising SEQ ID NO: 350.

8. (canceled)

9. An antibody or antigen-binding fragment thereof comprising a heavy chain variable region comprising SEQ ID NO: 378 (EVOLVESGGGLXQPGXSLRLSCXTSGFTLSDYYMSWVRQXPGKGLEWVXF MRNKANXYTTEYSASVRGRFTISRDTSKSXIYLQMNSLXXEDTAVYYCVRDR XGRPFAYWGQGTLVTVSS), wherein the X at position i (i=12, 16, 23, 40, 49, 57, 80, 89, 90 and 103) of SEQ ID NO: 378 is referred as X_{Hi}, wherein X_{H12} is V or I, X_{H16} is G or R, X_{H23} is A or T, X_{H40} is A or P, X_{H49} is G or S, X_{H57} is A or G, X_{H80} is I or T, X_{H89} is R or K, X_{H90} is A or T, X_{H103} is D or E; and
a light chain variable region comprising SEQ ID NO: 379 (DIXMTQSPXSLXXXXGXXXXIXCKSSQSLLNXINQKNFLTWYXQKPGXXPX LLIYWASTRESGVPXRFSGSGSGTDFTLXISXXXXEDLXXYYCQNDYTYPLTF GQGTKLEIK), wherein the X at position i (i=3, 9, 12, 13, 14, 15, 17, 18, 19, 20, 22, 32, 43, 48, 49, 51, 66, 80, 83, 84, 85, 86, 90 and 91) of SEQ ID NO: 379 is referred as X_{Li}, wherein X_{L3} is V or Q, X_{L9} is D, L or S, X_{L12} is A, S or P, X_{L13} is A or V, X_{L14} is S or T, X_{L15} is L, V or P, X_{L17} is D or E, X_{L18} is R or P, X_{L19} is A or V, X_{L20} is S or T, X_{L22} is N, T or S, X_{L32} is A or S, X_{L43} is Q or L, X_{L48} is Q or K, X_{L49} is A, P or S, X_{L51} is K or Q, X_{L66} is S or D, X_{L80} is K or T, X_{L83} is R or S, X_{L84} is L or V, X_{L85} is Q or E, X_{L86} is A or P, X_{L90} is A or G, X_{L91} is T or V.

10. An antibody or antigen-binding fragment thereof comprising a heavy chain variable region comprising SEQ ID NO: 380 (QVQLQESGPGLVKPSXTLSLTCXVXGYSITSDYAWNWIRQXPGKGLEWIGYI SHSGSTSYNPSLKSRVTISRDTSKNQFSLKLSSVTAADTAVYYCARSLGRRWY FDVWGQGTTVTVSS), wherein the X at position i (i=16, 23, 25 and 41) of SEQ ID NO: 380 is referred as X_{Hi}, wherein X_{H16} is E or Q, X_{H23} is A or T, X_{H25} is S or Y, X_{H41} is H or P; and
a light chain variable region comprising SEQ ID NO: 381 (DIXMTQSPXSLXXXXGXXXXIXCKSSQSLLXSSTQKNYLAWYXQKPGXXPX LLIYFASTRDSGVPXRFSGSGSGTDFTLXISXXXXEDLXXYFCQQHYIIPFTFG QGTKLEIK), wherein the X at position i (i=3, 9, 12, 13, 14, 15, 17, 18, 19, 20, 22, 31, 43, 48, 49, 51, 66, 80, 83, 84, 85, 86, 90 and 91) of SEQ ID NO: 381 is referred as X_{Li}, wherein X_{L3} is V or Q, X_{L9} is D, L or S, X_{L12} is A, S or P, X_{L13} is A or V, X_{L14} is S or T, X_{L15} is L, V or P, X_{L17} is D or E, X_{L18} is R or P, X_{L19} is A or V, X_{L20} is S or T, X_{L22} is N, T or S, X_{L31} is N or Q, X_{L43} is Q or L, X_{L48} is Q or K, X_{L49} is A, P or S, X_{L51} is K or Q, X_{L66} is S or D, X_{L80} is K or T, X_{L83} is R or S, X_{L84} is L or V, X_{L85} is Q or E, X_{L86} is A or P, X_{L90} is A or G, X_{L91} is T or V.

11. The antibody or antigen-binding fragment thereof of claim 9, which comprises:
a heavy chain variable region comprising 1, 2, or 3 CDR sequences shown as SEQ ID NO: 9, SEQ ID NO: 10 or 374 (MRNKANAYTT), and SEQ ID NO: 11 or 375 (VRDREGRPFAY), respectively; and a kappa light chain variable region comprising 1, 2, or 3 CDR sequences shown as SEQ ID NO: 12 or 376 (QSLLNAINQKNF), SEQ ID NO: 13, and SEQ ID NO: 14, respectively.

12. The antibody or antigen-binding fragment thereof of claim 9, which comprises a heavy chain variable region comprising a sequence selected from SEQ ID NO: 351, 353, 355, 357, 358, 360, 362 and 364; and a light chain variable region comprising a sequence selected from SEQ ID NO: 352, 354, 356, 359, 361 and 363.

13-14. (canceled)

15. The antibody or antigen-binding fragment thereof of claim 4, further comprising an immunoglobulin constant region, optionally a constant region of human Ig, or optionally a constant region of human IgG.

16-17. (canceled)

18. The antibody or antigen-binding fragment thereof of claim 4, which is a Fab, a Fab′, a F(ab′), a Fd, an Fv fragment, a disulfide stabilized Fv fragment (dsFv), a (dsFv)₂, a single-chain antibody molecule (scFv), a camelized single domain antibody, a nanobody, a domain antibody, and a bivalent domain antibody.

19. The antibody or antigen-binding fragment thereof of claim 4, wherein the antibody or antigen-binding fragment is linked to one or more conjugates, optionally, wherein the conjugate is covalently attached either directly or via a linker.

20-22. (canceled)

23. A pharmaceutical composition comprising the antibody or antigen-binding fragment thereof of claim 4, and a pharmaceutically acceptable carrier.

24-25. (canceled)

26. An isolated polynucleotide encoding the antibody or antigen-binding fragment thereof of claim 4.

27. A vector comprising the isolated polynucleotide of claim 26.

28. A host cell comprising the vector of claim 27.

29. (canceled)

30. A method of treating a disease or condition in a subject that would benefit from modulation of CD276 activity, comprising administering to the subject a therapeutically effective amount of the antibody or antigen-binding fragment thereof of claim 4.

31. (canceled)

32. The method of claim 30, wherein the disease or condition is cancer, adaptive immune disease, autoimmune disease, inflammatory disease, or infectious disease.

33-40. (canceled)

41. A method of modulating CD276 activity in a CD276-expressing cell, comprising exposing the CD276-expressing cell to the antibody or antigen-binding fragment thereof of claim 4.

42-67. (canceled)

68. The antibody or antigen-binding fragment thereof of claim 10, which comprises: a heavy chain variable region comprising 1, 2, or 3 CDR sequences shown as SEQ ID NO: 41, SEQ ID NO: 42, and SEQ ID NO: 43, respectively; and a kappa light chain variable region comprising 1, 2, or 3 CDR sequences shown as SEQ ID NO: 44 or 377 (QSLLOSSTOKNY), SEQ ID NO: 45, and SEQ ID NO: 46, respectively.

69. The antibody or antigen-binding fragment thereof of claim 10, which comprises a heavy chain variable region comprising a sequence selected from SEO ID NO: 365, 367 and 370; and a light chain variable region comprising a sequence selected from SEO ID NO: 366, 368, 369, 371, 372 and 373.
